(19) United States
(12) Patent Application Publication
(10) Pub. No.: US 2018/0271973 A1
(43) Pub. Date: Sep. 27, 2018

(54) COMPOSITIONS COMPRISING CH505 ENVELOPES, AND TRIMERS (EIGHT VALENT HIV-1 COMPOSITION AND METHODS)

(71) Applicants: Duke University, Durham, NC (US); Los Alamos National Security, LLC, Los Alamos, NM (US)

(72) Inventors: Barton F. HAYNES, Durham, NC (US); Hua-Xin LIAO, Durham, NC (US); Bette T. KORBER, Los Alamos, NM (US); Peter T. HRABER, Los Alamos, NM (US)

(21) Appl. No.: 15/539,573

(22) PCT Filed: Dec. 23, 2015
(86) PCT No.: PCT/US15/00222

§ 371 Date: Jun. 23, 2017

Related U.S. Application Data
(60) Provisional application No. 62/096,646, filed on Dec. 24, 2014.

(51) Int. Cl. A61K 39/21; C07K 14/16; A61P 31/18

(52) U.S. Cl. CPC A61K 39/21; A61K 2039/545; A61P 31/18; C07K 14/162

(57) ABSTRACT

In certain aspects the invention provides a selection of HIV-1 envelopes suitable for use as immunogens, and methods of using these immunogens in vaccination to induce neutralizing antibodies. In certain embodiments, the immunogens are designed to trimerize. In other embodiments, the immunogens and methods of using these comprise an immune modulating component.

This application claims the benefit of U.S. Application Ser. No. 62/096,646 filed Dec. 24, 2014, the entire content of which application is herein incorporated by reference.

### STATEMENT OF FEDERALLY FUNDED RESEARCH

This invention was made with government support under Center for HIV/AIDS Vaccine Immunology-Immunogen Design grant UM1-AI100645 from the NIH, NIAID, Division of AIDS. The government has certain rights in the invention.

All patents, patent applications and publications cited herein are hereby incorporated by reference in their entirety. The disclosure of these publications in their entireties are hereby incorporated by reference into this application in order to more fully describe the state of the art as known to those skilled therein as of the date of the invention described herein.

### FIELD OF THE INVENTION

The present invention relates in general, to a composition suitable for use in inducing anti-HIV-1 antibodies, and, in particular, to immunogenic compositions comprising envelope proteins and nucleic acids to induce cross-reactive neutralizing antibodies and increase their breadth of coverage. The invention also relates to methods of inducing such broadly neutralizing anti-HIV-1 antibodies using such compositions.

### BACKGROUND

The development of a safe and effective HIV-1 vaccine is one of the highest priorities of the scientific community working on the HIV-1 epidemic. While anti-retroviral treatment (ART) has dramatically prolonged the lives of HIV-1 infected patients, ART is not routinely available in developing countries.

### SUMMARY OF THE INVENTION

In certain aspects the invention provides a selection of HIV-1 envelopes, for example but not limited to M11, T/F Env, week 20.14, week 30.28, week 78.15, week 78.33, week 53.16, week 100.B6 Envs, or a combination thereof, for use in an HIV-1 vaccination scheme. In certain embodiments, the invention provides an immunization method wherein the selection of envelopes is administered sequentially and/or additively.

In certain aspects the invention provides a composition comprising any one of the polypeptides M11, T/F Env, week 20.14, week 30.28, week 78.15, week 78.33, week 53.16, week 100.B6 Envs, or a combination thereof. In certain aspects the invention provides a composition comprising any one of the polypeptides M11, T/F Env, week 20.14, week 30.28, week 78.15, week 78.33, week 53.16, week 100.B6 Envs, or a combination thereof.

In certain embodiments, the polypeptide comprises a trimerization domain. In certain embodiments the trimerziation domain is GCN4. In certain embodiments the trimerization domain is CD40L. In certain embodiments the trimerization domain is linked to the envelope sequence via a linker. In certain embodiments the linker is about 6 amino acids. In other embodiments the linker is about 3-20 amino acids. In certain embodiments, the linker is 3, 4, 5, 6, 7, 8; 9, 10, 11, 12, 13, 14, 15, 16, 17, 18, 19, 20 amino acids. In certain embodiments, the polypeptide further comprises a CD40L sequence.

In certain aspects the invention provides a composition comprising a nucleic acid encoding any one of the polypeptides of the invention.

In certain embodiments the HIV-1 envelopes are M11 and T/F Env. In certain embodiments the HIV-1 envelopes are week 20.14 and week 30.28. In certain embodiments, the HIV-1 envelopes are week 78.15 and week 78.33. In certain embodiments, the HIV-1 envelopes are week 53.16 and week 100.B6 Envs.

In certain embodiments the compositions of the invention further comprise an adjuvant.

In certain aspects the invention provides methods of inducing an immune response in a subject comprising administering the compositions of the invention in an amount sufficient to induce an immune response.

In certain aspects, the methods further comprise administering an immune modulating agent. In certain aspects, the methods further comprise administering chloloquine before each immunization. In certain embodiments, chloloquine is administered for about 10 days before each immunization.

In certain embodiments, the methods further comprise administering anti-CD25 antibody after each immunization, at an amount and duration sufficient to effect transient immunemodulation. In certain embodiments, the anti-CD25 antibody is administered for about 5 days before each immunization.

In certain embodiments, the methods further comprise administering anti-CD25 antibody after each immunization. In certain embodiments, anti-CD25 antibody is administered for about 5 days after each immunization.

In certain embodiments, the methods comprise administering a composition which comprises an immunogen as a nucleic acid, a protein or any combination thereof. In certain embodiments, the nucleic acid encoding the envelope is operably linked to a promoter inserted in an expression vector. In certain embodiments, the protein is recombinant.

In certain embodiments of the methods, the composition is administered as a prime, a boost, or both. In certain embodiments, the composition is administered as a multiple boosts.

In certain embodiments, the compositions further comprise an adjuvant.

In certain aspects the invention is directed to HIV-1 envelopes which are designed as fusion molecules comprising a portion of an envelope protein and a trimerization domain so as to trimerize. In certain aspects the invention is directed towards methods of using such HIV-1 envelopes for immunization so as to induce immune response, which comprises humoral immune response. In certain embodiments, the methods of immunization comprise administering an agent which transiently modulates the immune response.

In certain embodiments, the HIV-1 envelopes are administered as a nucleic acid, a protein or any combination thereof. In certain embodiments, the nucleic acid encoding the envelope is operably linked to a promoter inserted in an expression vector. In certain embodiments, the protein is recombinant.

In certain embodiments, the envelopes are administered as a prime, a boost, or both. In certain embodiments, the envelopes, or any combinations thereof are administered as a multiple boosts. In certain embodiments, the compositions and method further comprise an adjuvant. In certain embodiments, the HIV-1 envelopes are provided as nucleic acid sequences, including but not limited to nucleic acids optimized for expression in the desired vector and/or host cell. In other embodiments, the HIV-1 envelopes are provided as recombinantly expressed protein.

In certain embodiments, the invention provides compositions and method for induction of immune response, for example cross-reactive (broadly) neutralizing Ab induction. In certain embodiments, the methods use compositions comprising “swarms” of sequentially evolved envelope viruses that occur in the setting of bnAb generation in vivo in HIV-1 infection.

In certain aspects the invention provides compositions comprising a selection of HIV-1 envelopes or nucleic acids encoding these envelopes, for example but not limited to, as described herein. In certain embodiments, these compositions are used in immunization methods as a prime and/or boost, for example but not limited to, as described herein.

In certain embodiments, the compositions contemplate nucleic acid, as DNA and/or RNA, or protein immunogens either alone or in any combination. In certain embodiments, the methods contemplate genetic, as DNA and/or RNA, immunization either alone or in combination with envelope protein(s).

In certain embodiments the nucleic acid encoding an envelope is operably linked to a promoter inserted in an expression vector. In certain aspects the compositions comprise a suitable carrier. In certain aspects the compositions comprise a suitable adjuvant.

In certain embodiments the induced immune response includes induction of antibodies, including but not limited to autologous and/or cross-reactive (broadly) neutralizing antibodies against HIV-1 envelope. Various assays that analyze whether an immunogenic composition induces an immune response, and the type of antibodies induced are known in the art and are also described herein.

In certain aspects the invention provides an expression vector comprising any of the nucleic acid sequences of the invention, wherein the nucleic acid is operably linked to a promoter. In certain aspects the invention provides an expression vector comprising a nucleic acid sequence encoding any of the polypeptides of the invention, wherein the nucleic acid is operably linked to a promoter. In certain embodiments, the nucleic acids are codon optimized for expression in a mammalian cell, in vivo or in vitro. In certain aspects the invention provides nucleic acid comprising any one of the nucleic acid sequences of invention. A nucleic acid consisting essentially of any one of the nucleic acid sequences of invention. A nucleic acid consisting of any one of the nucleic acid sequences of invention. In certain embodiments the nucleic acid of invention, is operably linked to a promoter and is inserted in an expression vector. In certain aspects the invention provides an immunogenic composition comprising the expression vector.

In certain aspects the invention provides a composition comprising at least one of the nucleic acid sequences of the invention. In certain aspects the invention provides a composition comprising any one of the nucleic acid sequences of invention. In certain aspects the invention provides a composition comprising a combination of one nucleic acid sequence encoding any one of the polypeptides of the invention. In certain embodiments, combining DNA and protein gives higher magnitude of ab responses. See Pissani F. Vaccine 32: 507-13, 2013; Jalah R et al PLoS One 9: e91550, 2014.

In certain embodiments, the compositions and methods employ an HIV-1 envelope as polypeptide instead of a nucleic acid sequence encoding the HIV-1 envelope. In certain embodiments, the compositions and methods employ an HIV-1 envelope as polypeptide, a nucleic acid sequence encoding the HIV-1 envelope, or a combination thereof. The envelope can be a gp160, gp150, gp140, gp120, gp41, N-terminal deletion variants as described herein, cleavage resistant variants as described herein, or codon optimized sequences thereof. The polypeptide of the inventions can be a trimer. The polypeptide contemplated by the invention can be a polypeptide comprising any one of the polypeptides described herein. The polypeptide contemplated by the invention can be a polypeptide consisting essentially of any one of the polypeptides described herein. The polypeptide contemplated by the invention can be a polypeptide consisting of any one of the polypeptides described herein. In certain embodiments, the polypeptide is recombinantly produced. In certain embodiments, the polypeptides and nucleic acids of the invention are suitable for use as an immunogen, for example to be administered in a human subject.

### BRIEF DESCRIPTION OF THE DRAWINGS

To conform to the requirements for PCT patent applications, many of the figures presented herein are black and white representations of images originally created in color. In the below descriptions and the examples, the colored images are described in terms of its appearance in black and white.

FIG. 1 shows designs of HIV-1 envelopes with trimerization domain, and immune modulating (e.g. CD40L) domain.

FIGS. 2A and 2B show Envelope monomer trimer QC—Non-reducing conditions.

FIGS. 3A and 3B show Envelope monomer trimer QC—reducing conditions.

FIG. 4 shows Envelope trimer by Blue Native PAGE.

FIG. 5 shows gp120 trimers antigenicity. FIG. 5 is a table with a summary of the data in FIGS. 6-11. FIG. 5 shows that CH505TF gp120 GCN4 Trimer binds to CH103 UCA (CD4bs) with a lower Kd (nm) compared to the CH505TF gp120 D7 Monomer.

FIG. 6 shows gp120 trimers antigenicity. The upregulation by sCD4 on CH505TFgp120GCN4 293i Trimer Batch#140826 is shown.

FIGS. 7-11 show gp120 trimers antigenicity.

FIG. 12 shows design of Design of CD40L-MPER656 peptide-liposome conjugate.

FIGS. 13A-13C shows antigenicity of the MPER-liposome of FIG. 12. Biolayer interferometry assay of binding of mouse anti-human CD40L mAb (FIG. 13A) and broadly neutralizing HIV-1 gp41 MPER mAbs 2F5 (FIG. 13B) and 4E10 (FIG. 13C) at 20 μg/ml to CD40L-MPER656 liposomes loaded onto Aminopropyl silane sensors are shown. The binding of antibodies to appropriate control liposomes were subtracted to obtain the specific binding shown in FIGS. 13A-13C.

FIG. 14 shows that the CD40L-MPER656 peptide-liposome conjugate is functional. Human CD40 expressing HEK blue cells are activated by CD40L-MPER656 liposome. The line and circle designated (1) correspond to His6-hCD40L-MPER656 liposomes. The line and circle designated (2) correspond to His10-GCN4-L11-hCD40L-MPER656 liposomes. The line and circle designated (3) correspond to IgL-GCN4-L11-CD40L-His10-MPER656 liposomes.

FIG. 15 shows that CH505 gp120-GCN4-CD40L activates human CD40 expressing HEK cells. Both the Env constructs (with and without His tag) were active. Liposome conjugation did not enhance the activity of His tagged CH505 gp120-GCN4-CD40L construct. The Env without CD40L is not active showing that the CD40 activation by these constructs is CD40L mediated. The line and circle designated (1) correspond to CH505 gp120-GCN4-hCD40L. The line and circle designated (2) correspond to CH505 gp120-GCN4-hCD40L-10His. The line and circle designated (3) correspond to CH505 gp120-GCN4-hCD40L-10His liposomes. The line and circle designated (4) correspond to CH505 gp120-GCN4.

FIG. 16 shows antigenicity of CH505 120-GCN4-CD40L. SPR binding assay essentially as described in FIG. 13.

FIG. 17 shows the sequences of a selection of ten envelopes (“P10” derived from CH505). The nucleotide sequences for the following GP120 DNA constructs are shown: HV1300532_v2, CH505.M6D8gp120 (SEQ ID NO.: 1), HV1300537_v2, CH505.M11D8gp120(SEQ ID NO.: 2), HV1300556_v2, CH505w020.14D8gp120 (SEQ ID NO.: 3), HV1300578_v2, CH505w030.28D8gp120 (SEQ ID NO.: 4), HV1300574_v2, CH505w030.21D8gp120 (SEQ ID NO.: 5), HV1300583, CH505w053.16D8gp120 (SEQ ID NO.: 6), HV1300586, CH505w053.31D8gp120 (SEQ ID NO.: 7), HV1300595, CH505w078.33D8gp120 (SEQ ID NO.: 8), HV1300592, CH505w078.15D8gp120 (SEQ ID NO.: 9), HV1300605, CH505w100.B6D8gp120 (SEQ ID NO.: 10). The amino acid sequences of the production 10 CH505 Δ8gp120 are shown: HV1300532_v2, CH505.M6D8gp120 (SEQ ID NO.: 11), HV1300537_v2, CH505.M11D8gp120 (SEQ ID NO.: 12), HV1300556_v2, CH505w020.14D8gp120 (SEQ ID NO.: 13), HV1300578_v2, CH505w030.28D8gp120 (SEQ ID NO.: 14), HV1300574_v2, CH505w030.21D8gp120 (SEQ ID NO.: 15), HV1300583, CH505w053.16D8gp120 (SEQ ID NO.: 16), HV1300586, CH505w053.31D8gp120 (SEQ ID NO.: 17), HV1300595, CH505w078.33D8gp120 (SEQ ID NO.: 18), HV1300592, CH505w078.15D8gp120 (SEQ ID NO.: 19), HV1300605, CH505w100.B6D8gp120 (SEQ ID NO.: 20). The nucleotide sequences for the following Gp145 DNA constructs are shown: HV1300657 (SEQ ID NO.: 21), HV1300662 (SEQ ID NO.: 22), HV1300635 (SEQ ID NO.: 23), HV1300636 (SEQ ID NO.: 24), HV1300689 (SEQ ID NO.: 25), HV1300696 (SEQ ID NO.: 26), HV1300638 (SEQ ID NO.: 27), HV1300705 (SEQ ID NO.: 28), HV1300639 (SEQ ID NO.: 29), HV1300714 (SEQ ID NO.: 30). The nucleotide sequences for the following Gp160 constructs are shown: CH505.M6 (SEQ ID NO.: 31), CH505.M11 gp160 (SEQ ID NO.: 32), CH505w020.14 gp160 (SEQ ID NO.: 33), CH505w030.28 gp160 (SEQ ID NO.: 34), CH505w030.21 gp160 (SEQ ID NO.: 35), CH505w053.16 gp160 (SEQ ID NO.: 36), CH505w053.31 gp160 (SEQ ID NO.: 37), CH505w078.33 gp160 (SEQ ID NO.: 38), CH505w078.15 gp160 (SEQ ID NO.: 39), CH505w100.B6 gp160 (SEQ ID NO.: 40). The following GP160 amino acid sequences are shown: CH505.M6 gp160 (SEQ ID NO.: 41), CH505.M11 gp160 (SEQ ID NO.: 42), CH505w020.14 gp160 (SEQ ID NO.: 43), CH505w030.28 gp160 (SEQ ID NO.: 44), CH505w030.21 gp160 (SEQ ID NO.: 45), CH505w053.16 gp160 (SEQ ID NO.: 46), CH505w053.31 gp160 (SEQ ID NO.: 47), CH505w078.33 gp160 (SEQ ID NO.: 48), CH505w078.15 gp160 (SEQ ID NO.: 49), CH505w100.B6 gp160 (SEQ ID NO.: 50).

FIG. 18 shows binding of CH103 antibodies to the autologous Envs of FIG. 17, log AUC.

FIG. 19 shows neutralization IC50s of CH103 lineage mAbs against autologous CH505 Envs. Pseudoviruses are sorted by sensitivity to CH103-lineage mAbs, then geometric mean IC50. Here only 108 viruses with distinct gp120s are shown, not the full set of 135 Envs assayed.

FIGS. 20A-20E shows autologous neutralization profiles for (FIG. 20A) mutated TF viruses, (FIG. 20B) 4-Env immunogen set, (FIG. 20C) previously identified 10-Env immunogen set, and (FIG. 20D) currently identified 10-Env immunogen set. FIG. 20E shows the sequences corresponding to FIGS. 20A-D. Concatenated sites listed in Table 6 are shown for each candidate immunogen.

FIGS. 21A-21C show (FIG. 21A) Env Mutations, (FIG. 21B) CH103 lineage MAb IC50s, and (FIG. 21C) Env phylogeny for CH505. Env immunogens proposed in alternative vaccination regimes are shown by colored diamonds. Unlike earlier phylogenies of these Envs, indels here are treated as distinct characters, rather than missing data.

FIG. 22 shows the amino acid sequences of TF, Week 78.33, Week 53.16, Week 100.B6 HIV-1 envelopes. The sequences of a selection of four CH505 envelopes: CH505w000.TFgp160 (SEQ ID NO.: 51), CH505w053.16gp160 (SEQ ID NO.: 52), CH505w078.33gp160 (SEQ ID NO.: 53), CH505w100.B6gp160 (SEQ ID NO.: 54).

FIG. 23 shows the amino acid sequence (SEQ ID NO.: 55) and nucleic acid sequence of CAP-206 6m HIV-1 envelope (SEQ ID NO.: 56).

FIG. 24 shows the amino acid sequences of envelopes of FIG. 20 D: CH505M11gp160 (SEQ ID NO.: 57), CH505w004.03gp160 (SEQ ID NO.: 58), CH505w020.14gp160 (SEQ ID NO.: 59), CH505w030.28gp160 (SEQ ID NO.: 60), CH505w30.12 (SEQ ID NO.: 61), CH505w020.2 (SEQ ID NO.: 62), CH505w030.10gp160 (SEQ ID NO.: 63), CH505w078.15gp160 (SEQ ID NO.: 64), CH505w030.19gp160 (SEQ ID NO.: 65), CH505w030.21gp160 (SEQ ID NO.: 66).

FIG. 25 shows the steps of a B Cell Lineage-Based Approach to Vaccine Design.

FIG. 26 shows the HIV-1 Arms Race: Isolation of Broad Neutralizing Antibodies From Chronically Infected Individual CH0505 Followed From Time of Transmission.

FIG. 27 shows a Heat Map of Binding (log Area Under the Curve, AUC) of Sequential Envs to CH103 CD4 Binding Site Broadly Neutralizing Antibody Lineage Members.

FIG. 28 shows the Binding Specificities of HIV-1 CH505 Env-induced Antibodies (NHP79).

FIG. 29 shows HIV-1 Binding and Neutralization Profiles of Rhesus Monoclonal Antibody, DH359.

FIG. 30 shows the HIV-1 Arms Race: Isolation of Broad Neutalizing Antibodies From Chronically Infected Individual CH505 Followed From Time of Transmission.

FIG. 31 shows the Cooperation of B Cell Lineages in Induction of HIV-1 Broad Neutralizing Antibodies.

FIG. 32A shows a Screen of 33 Envs for Binding to CH103 bnAbs Lineage Antibody Member. FIG. 32B shows the sequences corresponding to FIG. 32A.

FIG. 33 shows a Heat Map of Binding (log Area Under the Curve, AUC) of Sequential Envs to CH103 CD4 Binding Site Broadly Neutralizing Antibody Lineage Members.

### DETAILED DESCRIPTION

The development of a safe, highly efficacious prophylactic HIV-1 vaccine is of paramount importance for the control and prevention of HIV-1 infection. A major goal of HIV-1 vaccine development is the induction of broadly neutralizing antibodies (bnAbs) (Immunol. Rev. 254: 225-244, 2013). BnAbs are protective in rhesus macaques against SHIV challenge, but as yet, are not induced by current vaccines.

For the past 25 years, the HIV vaccine development field has used single or prime boost heterologous Envs as immunogens, but to date has not found a regimen to induce high levels of bnAbs.

Recently, a new paradigm for design of strategies for induction of broadly neutralizing antibodies was introduced, that of B cell lineage immunogen design (Nature Biotech. 30: 423, 2012) in which the induction of bnAb lineages is recreated. It was recently demonstrated the power of mapping the co-evolution of bnAbs and founder virus for elucidating the Env evolution pathways that lead to bnAb induction (Nature 496: 469, 2013). From this type of work has come the hypothesis that bnAb induction will require a selection of antigens to recreate the “swarms” of sequentially evolved viruses that occur in the setting of bnAb generation in vivo in HIV infection (Nature 496: 469, 2013).

A critical question is why the CH505 immunogens are better than other immunogens. This rationale comes from three recent observations. First, a series of immunizations of single putatively “optimized” or “native” trimers when used as an immunogen have not induced bnAbs as single immunogens. Second, in all the chronically infected individuals who do develop bnAbs, they develop them in plasma after ˜2 years. When these individuals have been studied at the time soon after transmission, they do not make bnAbs immediately. Third, now that individual's virus and bnAb co-evolution has been mapped from the time of transmission to the development of bnAbs, the identification of the specific Envs that lead to bnAb development have been identified-thus taking the guess work out of env choice.

Two other considerations are important. The first is that for the CH103 bnAb CD4 binding site lineage, the VH4-59 and Vλ3-1 genes are common as are the VDJ, VJ recombinations of the lineage (Liao, Nature 496: 469, 2013). In addition, the bnAb sites are so unusual, we are finding that the same VH and VL usage is recurring in multiple individuals. Thus, we can expect the CH505 Envs to induce CD4 binding site antibodies in many different individuals.

Finally, regarding the choice of gp120 vs. gp160, for the genetic immunization we would normally not even consider not using gp160. However, in acute infection, gp41 non-neutralizing antibodies are dominant and overwhelm gp120 responses (Tomaras, G et al. J. Virol. 82: 12449, 2008; Liao, H X et al. JEM 208: 2237, 2011). Recently we have found that the HVTN 505 DNA prime, rAd5 vaccine trial that utilized gp140 as an immunogen, also had the dominant response of non-neutralizing gp41 antibodies. Thus, we will evaluate early on the use of gp160 vs gp120 for gp41 dominance.

In certain aspects the invention provides a strategy for induction of bnAbs is to select and develop immunogens designed to recreate the antigenic evolution of Envs that occur when bnAbs do develop in the context of infection.

That broadly neutralizing antibodies (bnAbs) occur in nearly all sera from chronically infected HIV-1 subjects suggests anyone can develop some bnAb response if exposed to immunogens via vaccination. Working back from mature bnAbs through intermediates enabled understanding their development from the unmutated ancestor, and showed that antigenic diversity preceded the development of population breadth. See Liao et al. (2013) Nature 496, 469-476. In this study, an individual “CH505” was followed from HIV-1 transmission to development of broadly neutralizing antibodies. This individual developed antibodies targeted to CD4 binding site on gp120. In this individual the virus was sequenced over time, and broadly neutralizing antibody clonal lineage (“CH103”) was isolated by antigen-specific B cell sorts, memory B cell culture, and amplified by VH/VL next generation pyrosequencing. See Liao et al. (2013) Nature 496, 469-476.

Further analysis of envelopes and antibodies from the CH505 individual indicated that a non-CH103 Lineage participates in driving CH103-BnAb induction. For example V1 loop, V5 loop and CD4 binding site loop mutations escape from CH103 and are driven by CH103 lineage. Loop D mutations enhanced neutralization by CH103 lineage and are driven by another lineage. Transmitted/founder Env, or another early envelope for example W004.03, and/or W004.26, triggers naïve B cell with CH103 Unmutated Common Ancestor (UCA) which develop in to intermediate antibodies. Transmitted/founder Env, or another early envelope for example W004.03, and/or W004.26, also triggers non-CH103 autologous neutralizing Abs that drive loop D mutations in Env that have enhanced binding to intermediate and mature CH103 antibodies and drive remainder of the lineage.

The invention provides various methods to choose a subset of viral variants, including but not limited to envelopes, to investigate the role of antigenic diversity in serial samples. In other aspects, the invention provides compositions comprising viral variants, for example but not limited to envelopes, selected based on various criteria as described herein to be used as immunogens.

In other aspects, the invention provides immunization strategies using the selections of immunogens to induce cross-reactive neutralizing antibodies. In certain aspects, the immunization strategies as described herein are referred to as “swarm” immunizations to reflect that multiple envelopes are used to induce immune responses. The multiple envelopes in a swarm could be combined in various immunization protocols of priming and boosting.

Sequences/Clones

Described herein are nucleic and amino acids sequences of HIV-1 envelopes. In certain embodiments, the described HIV-1 envelope sequences are gp160s. In certain embodiments, the described HIV-1 envelope sequences are gp120s. Other sequences, for example but not limited to gp140s, both cleaved and uncleaved, gp150s, gp41s, which are readily derived from the nucleic acid and amino acid gp160 sequences. In certain embodiments the nucleic acid sequences are codon optimized for optimal expression in a host cell, for example a mammalian cell, a rBCG cell or any other suitable expression system.

In certain embodiments, the envelope design in accordance with the present invention involves deletion of residues (e.g., 5-11, 5, 6, 7, 8, 9, 10, or 11 amino acids) at the N-terminus. For delta N-terminal design, amino acid residues ranging from 4 residues or even fewer to 14 residues or even more are deleted. These residues are between the maturation (signal peptide, usually ending with CX, X can be any amino acid) and “VPVXXXX . . . ”. In case of CH505 T/F Env as an example, 8 amino acids (italicized and underlined in the below sequence) were deleted: MRVMGIQRNYPQWWIWSMLGFWMLMICNGMWVTVYYGVPVWKEAKTTLFCASDAK AYEKEVHNVWATHACVPTDPNPQE . . . (rest of envelope sequence is indicated as “ . . . ”). In other embodiments, the delta N-design described for CH505 T/F envelope can be used to make delta N-designs of other CH505 envelopes. In certain embodiments, the invention relates generally to an immunogen, gp160, gp120 or gp140, without an N-terminal Herpes Simplex gD tag substituted for amino acids of the N-terminus of gp120, with an HIV leader sequence (or other leader sequence), and without the original about 4 to about 25, for example 11, amino acids of the N-terminus of the envelope (e.g. gp120). See WO2013/006688, e.g. at pages 10-12, the contents of which publication is hereby incorporated by reference in its entirety.

The general strategy of deletion of N-terminal amino acids of envelopes results in proteins, for example gp120s, expressed in mammalian cells that are primarily monomeric, as opposed to dimeric, and, therefore, solves the production and scalability problem of commercial gp120 Env vaccine production. In other embodiments, the amino acid deletions at the N-terminus result in increased immunogenicity of the envelopes.

In certain embodiments, the invention provides envelope sequences, amino acid sequences and the corresponding nucleic acids, and in which the V3 loop is substituted with the following V3 loop sequence TRPNNNTRKSIRIGPGQTFY ATGDIIGNIRQAH. This substitution of the V3 loop reduced product cleavage and improves protein yield during recombinant protein production in CHO cells.

In certain embodiments, the CH505 envelopes will have added certain amino acids to enhance binding of various broad neutralizing antibodies. Such modifications could include but not limited to, mutations at W680G or modification of glycan sites for enhanced neutralization.

In certain aspects, the invention provides composition and methods which use a selection of sequential CH505 Envs, as gp120s, gp 140s cleaved and uncleaved and gp160s, as proteins, DNAs, RNAs, or any combination thereof, administered as primes and boosts to elicit immune response. Sequential CH505 Envs as proteins would be co-administered with nucleic acid vectors containing Envs to amplify antibody induction.

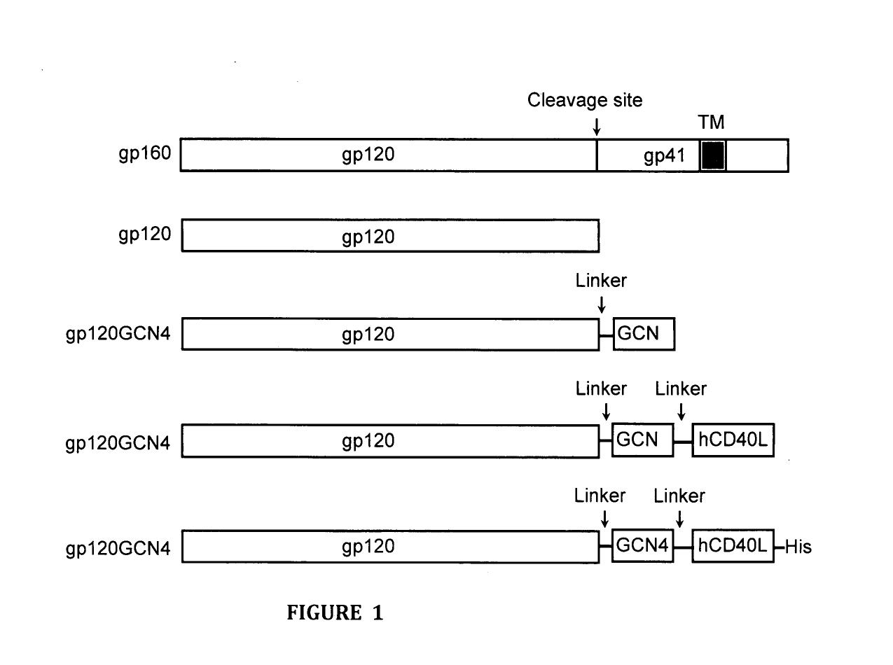

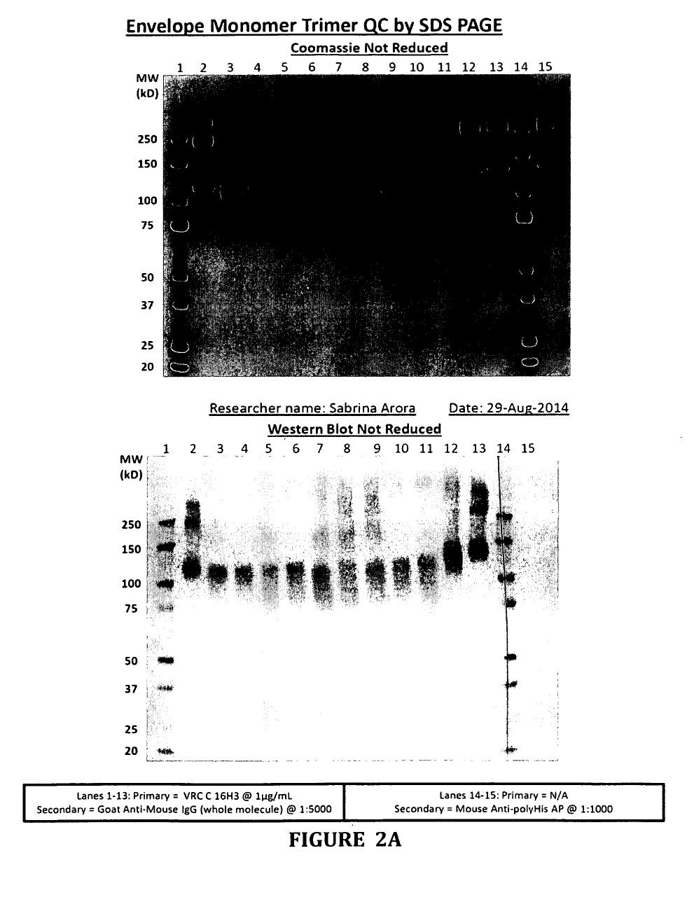

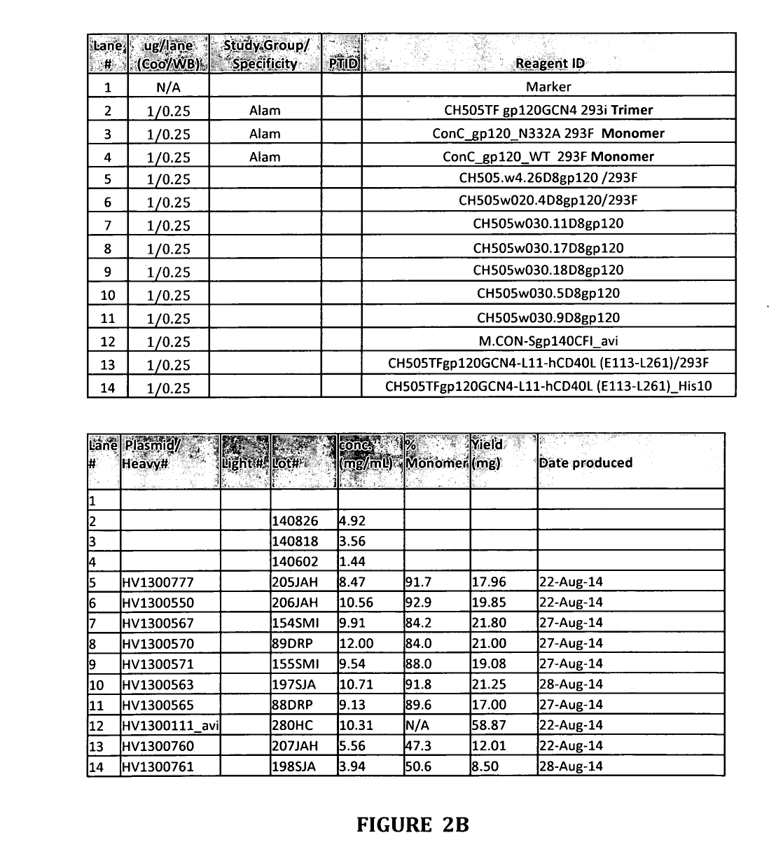

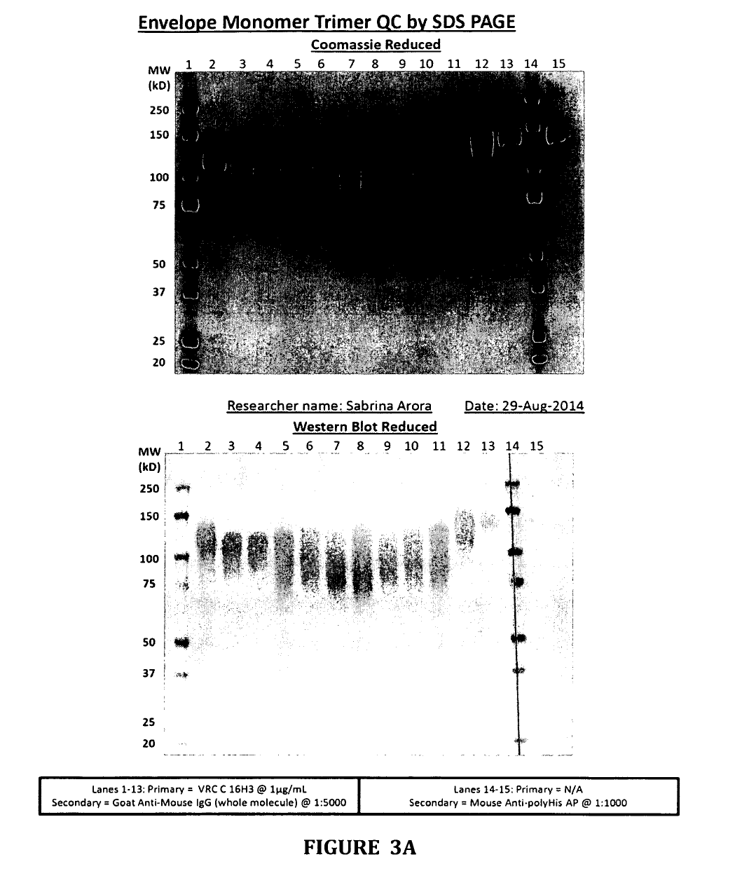

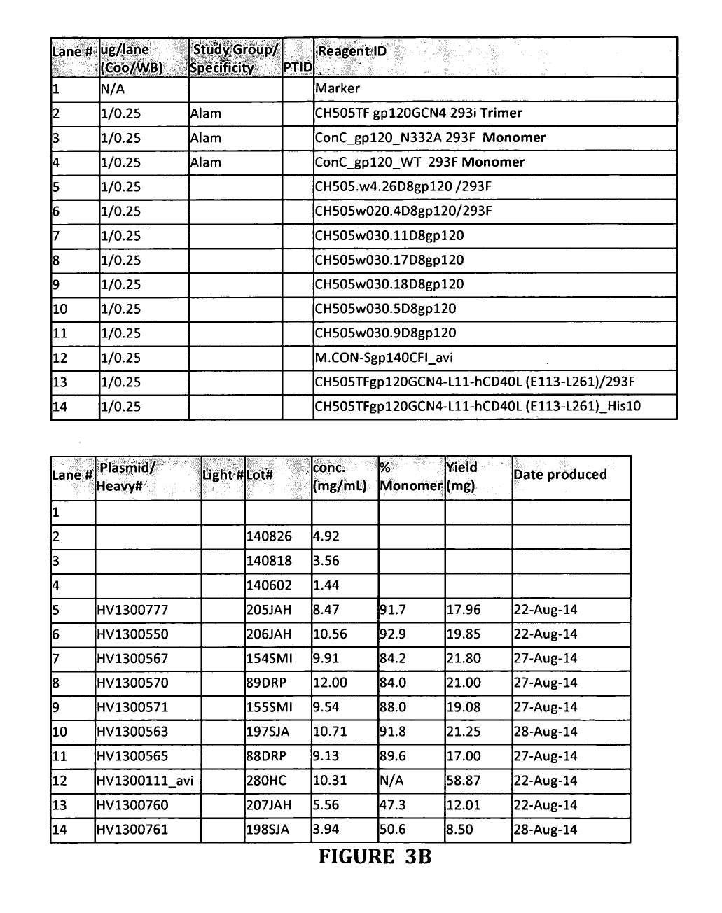

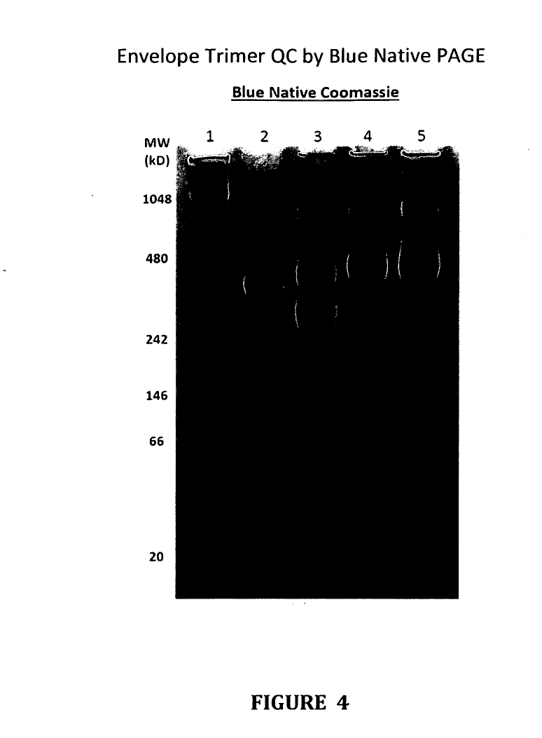

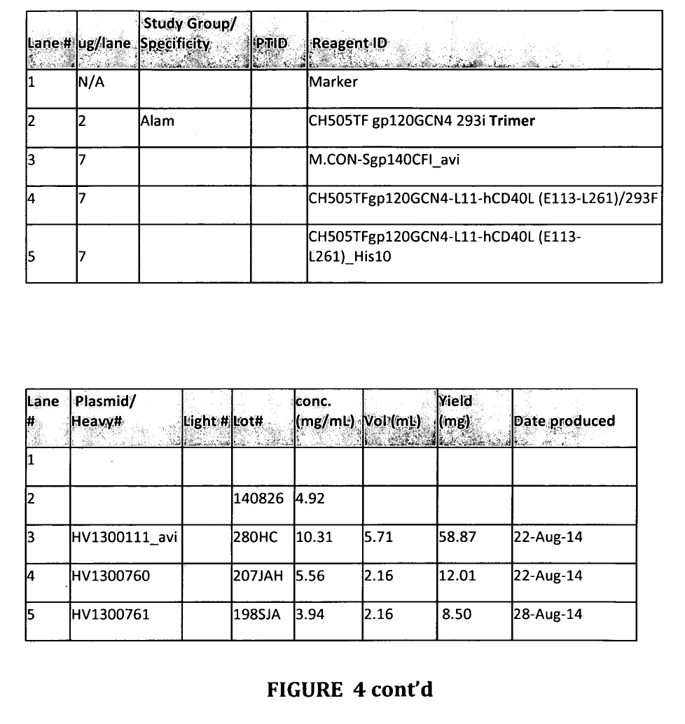

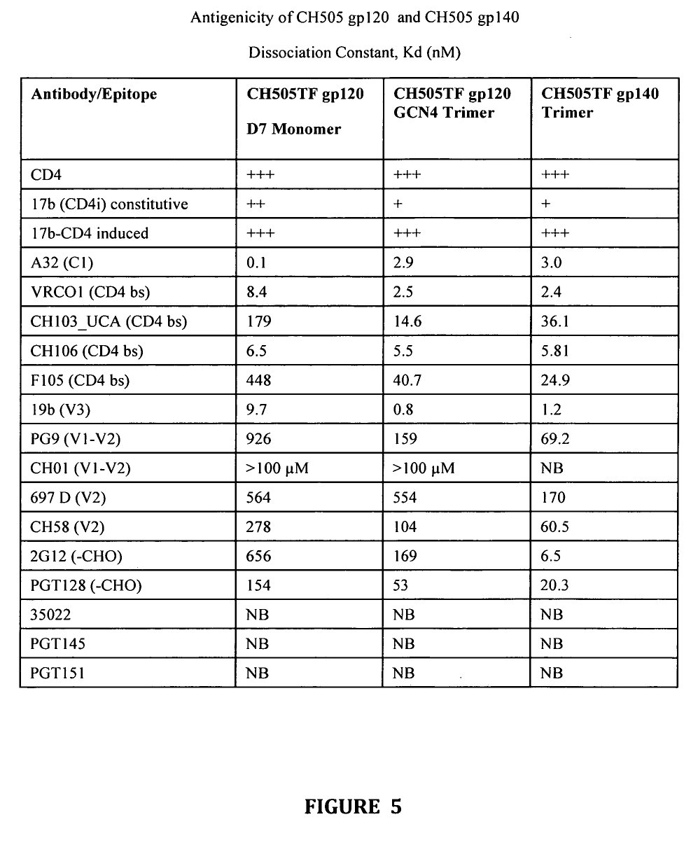

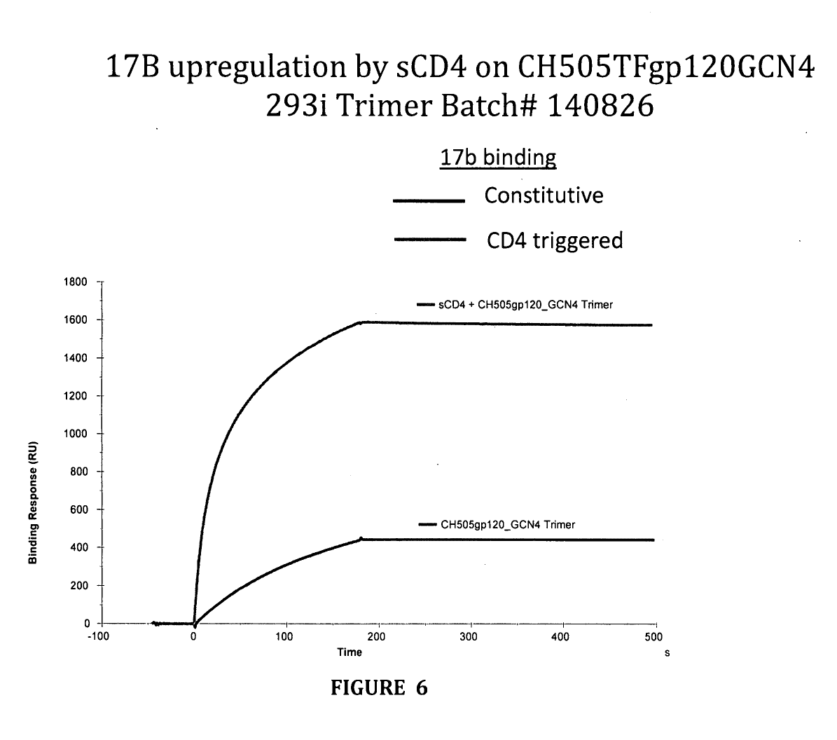

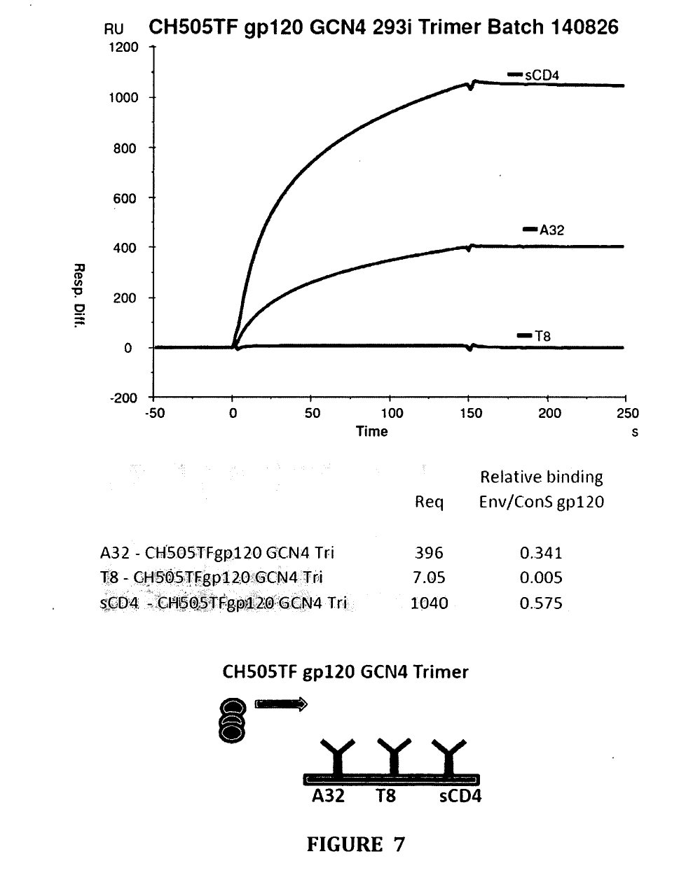

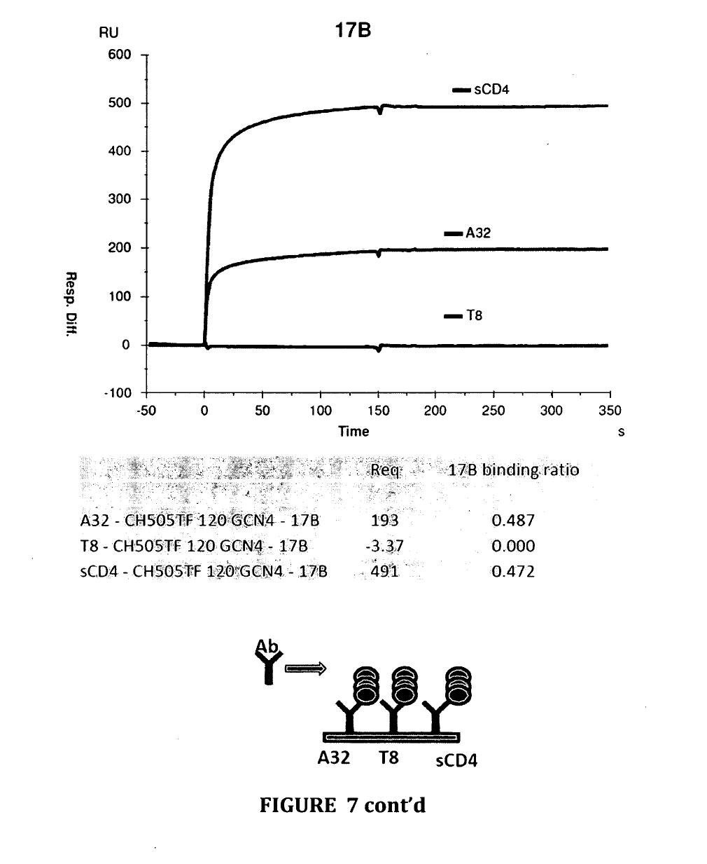

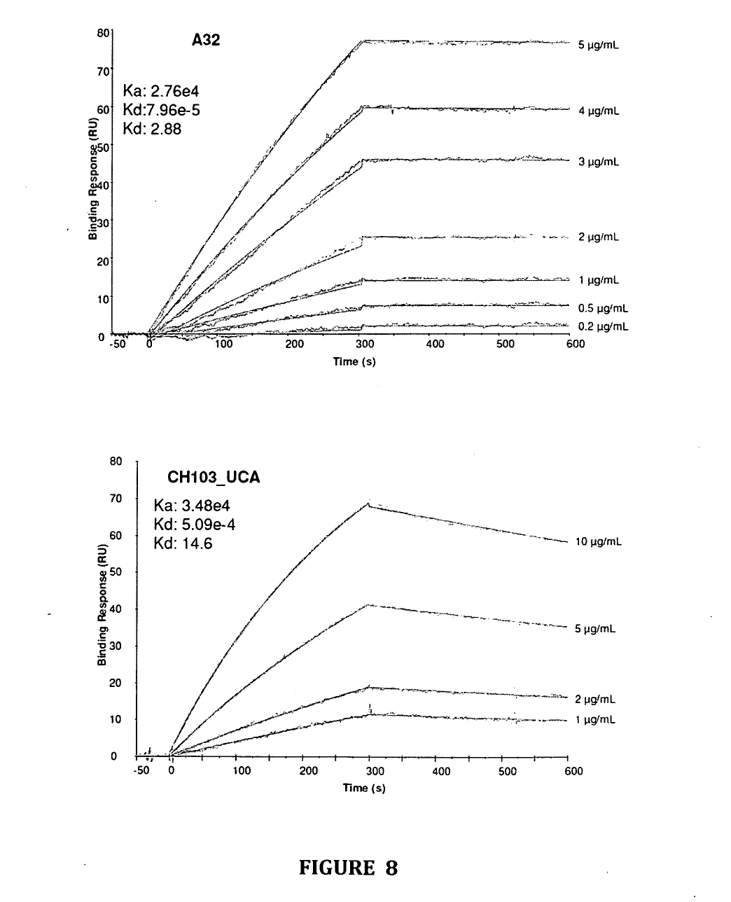

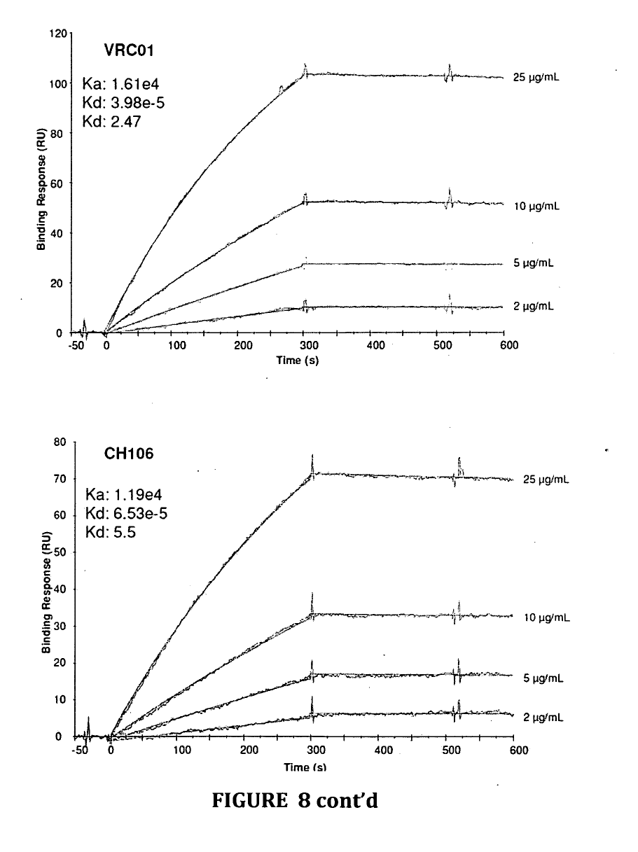

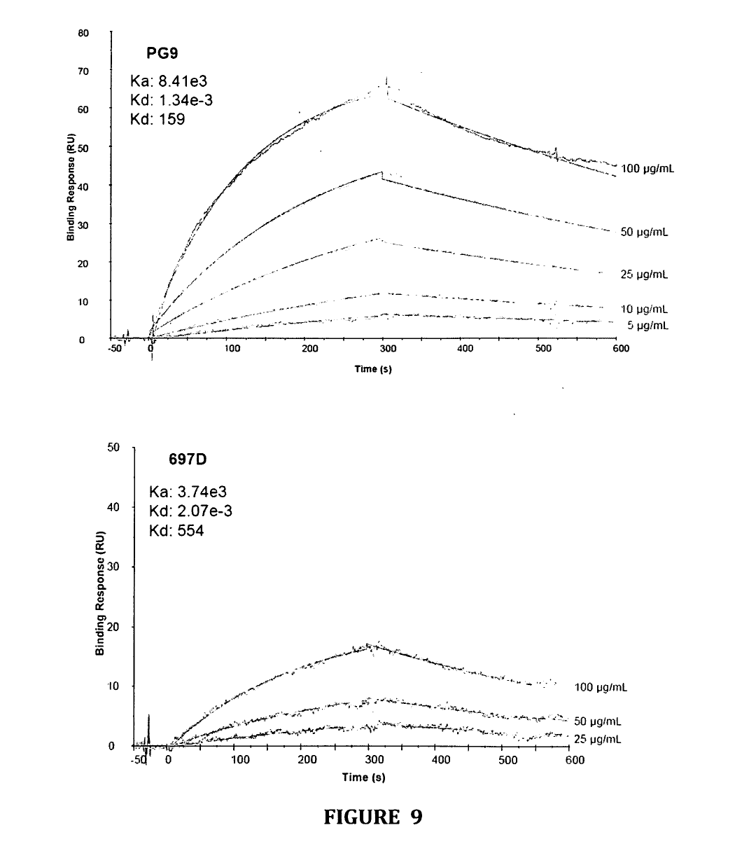

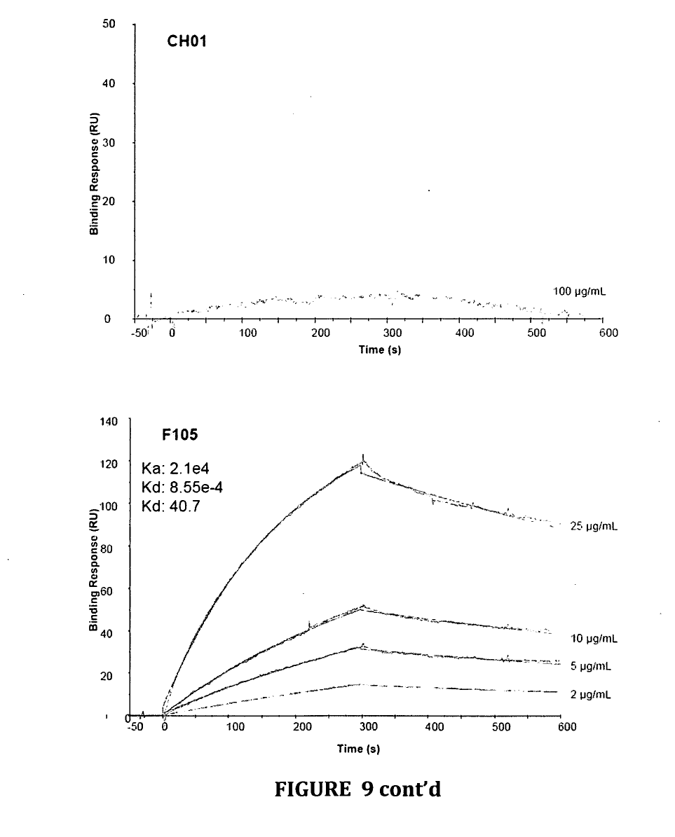

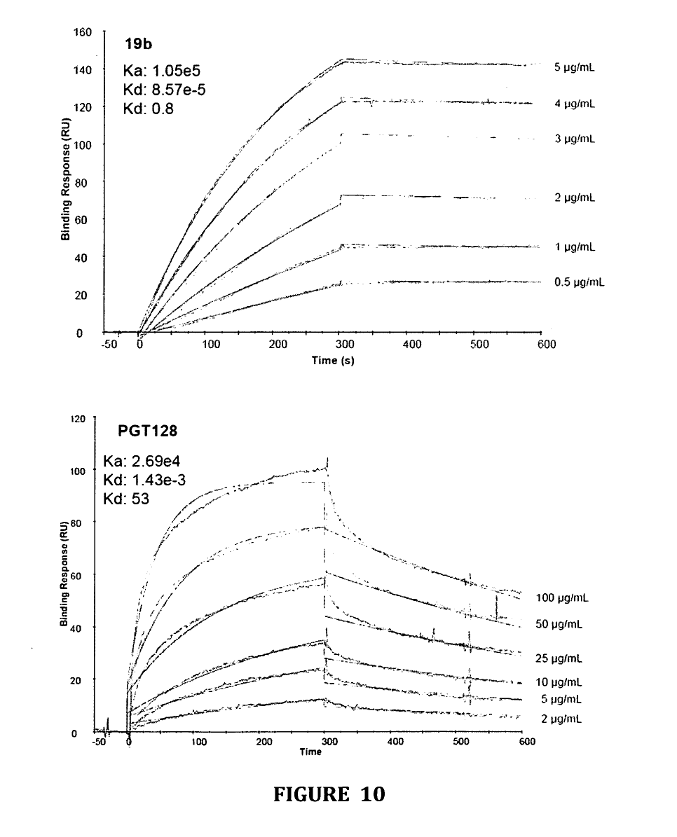

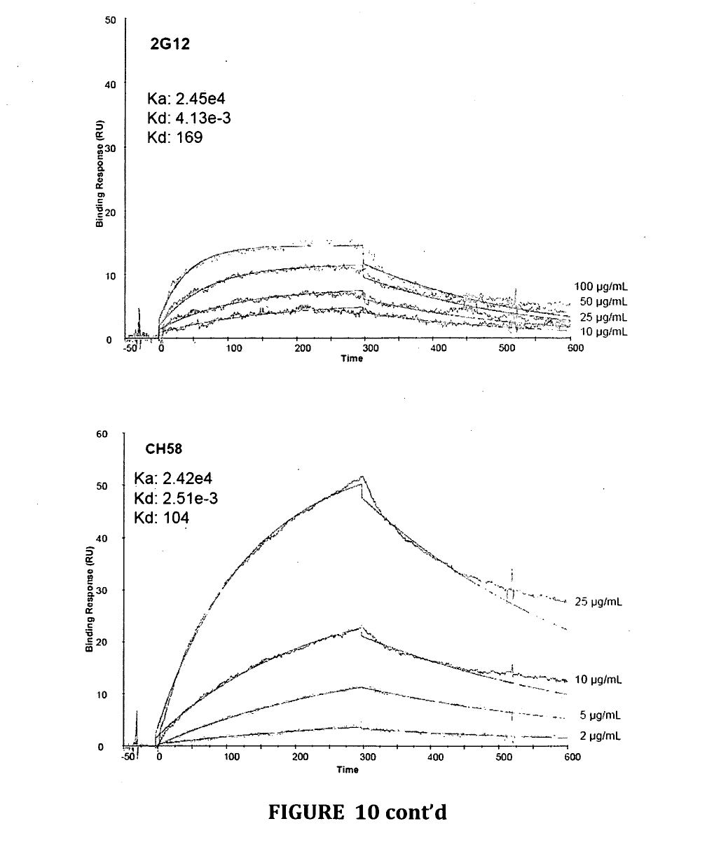

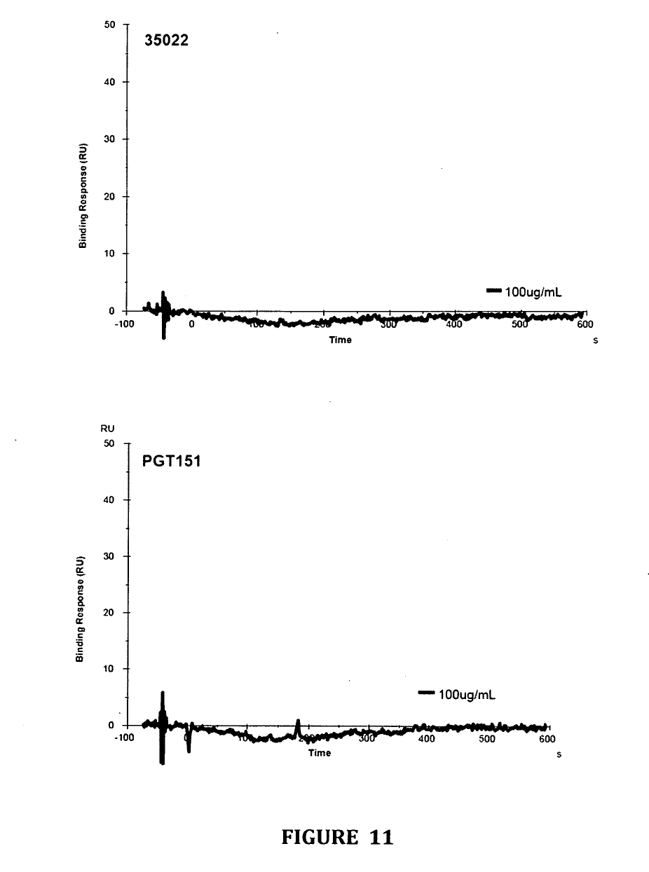

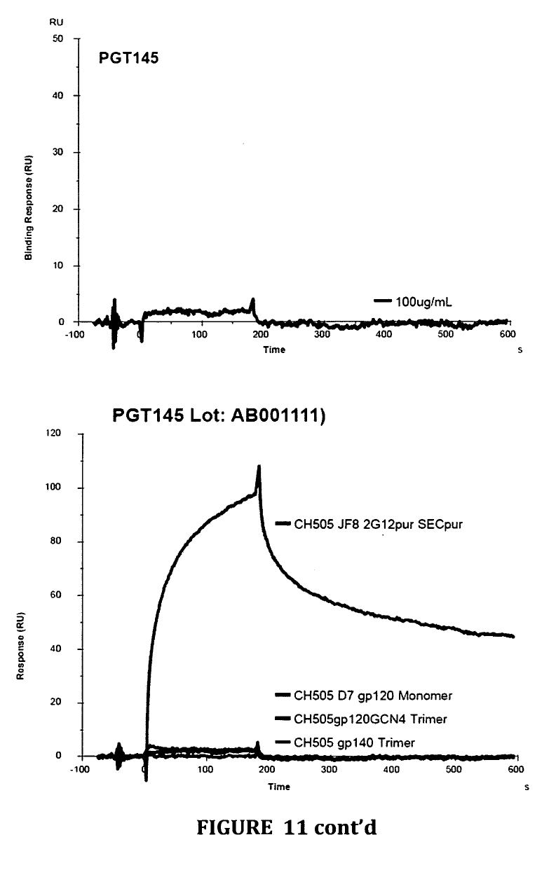

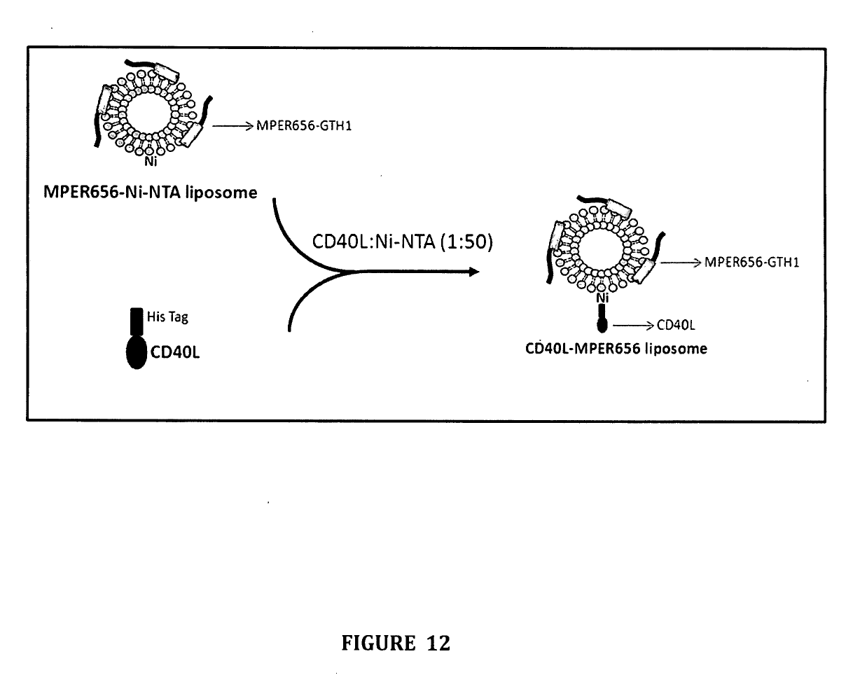

In certain embodiments the invention provides immunogens and compositions which include immunogens as trimers. In certain embodiments, the immunogens include a trimerization domain which is not derived from the HIV-1 envelope. In certain embodiments, the trimerization domain is GCN4 (See FIG. 1). In other embodiments the trimerization is CD40L. In other embodiments, the immunogens include CD40L domain (See FIGS. 1 and 12).

HIV-1 gp120 Trimer Vaccine Immunogens (FIG. 1):

HIV-1 Env Gp120 GCN4 Trimer

HIV-1 Env gp120 GCN4 trimer is designed to be expressed as soluble recombinant trimeric HIV-1 gp120 protein. HIV-1 Env gp120 is mutated from residue R to E at the cleavage site of HIV-1 gp120 at the residue positions R503 and R511 (or any mutations at this region) to destroyed the cleavage site, a 6-residue linker (GSGSGS) (the linker can be variations of 3-20 residues in length) is added to the C-terminal end of HIV-1 gp120 followed by addition of 33 amino acid residues of GCN4 sequence (RMKQIEDKIEEILSKIYHIENEIARIKKLIGER).

HIV-1 Env Gp120 GCN4 CD40L Trimer:

In certain embodiments the trimer design includes an immune co-stimulator HIV-1 Env gp120 GCN4 CD40L trimer is designed to be expressed as soluble recombinant trimeric HIV-1 gp120 protein co-expressed with functional CD40L as immune co-stimulator. HIV-1 Env gp120 is mutated from residue R to E at the cleavage site of HIV-1 gp120 at the residue positions R503 and R511 (or any mutations at this region) to destroy the cleavage site, a 6-residue linker (GSGSGS) (the linker can be variations of 3-20 residues in length) is added to the C-terminal end of HIV-1 gp120, 33 amino acid residues of GCN4 sequence (RMKQIEDKIEEILSKIYHIENEIARIKKLIGER) is added to the C terminal end of the 6-residue linker, then a 11-residue liner (GGSGGSGGSGG) (the linker can be variations of 3-20 residues in length) is added to the C terminal end of the GCN4 domain, followed by addition of the sequence of the functional extracellular domain of the human CD40 ligand (L) E113-L261.

HIV-1 Env Gp120 GCN4 CD40L Trimer with His Tag:

HIV-1 Env gp120 GCN4 CD40L trimer with His tag is designed to be expressed as soluble recombinant trimeric HIV-1 gp120 protein co-expressed with functional CD40L as immune co-stimulator. HIV-1 Env gp120 is mutated from residue R to E at the cleavage site of HIV-1 gp120 at the residue positions R503 and R511 (or any mutations at this region) to destroyed the cleavage site, a 6-residue linker (GSGSGS) (the linker can be variations of 3-20 residues in length) is added to the C-terminal end of HIV-1 gp120, 33 amino acid residues of GCN4 sequence (RMKQIEDKIEEILSKIYHIENEIARIKKLIGER) is added to the C terminal end of the 6-residue linker, a 11-residue liner (GGSGGSGGSGG) (the linker can be variations of 3-20 residues in length) is added to the C terminal end of the GCN4 domain, then the sequence of the functional extracellular domain of the human CD40 ligand (L) E113-L261 is then added followed by addition of 10 histine residues as tag (the His tag can be more or less of 10 residues). His-tag is added to anchor the HIV-1 gp120GCN4 CD40L into liposome through nickel.

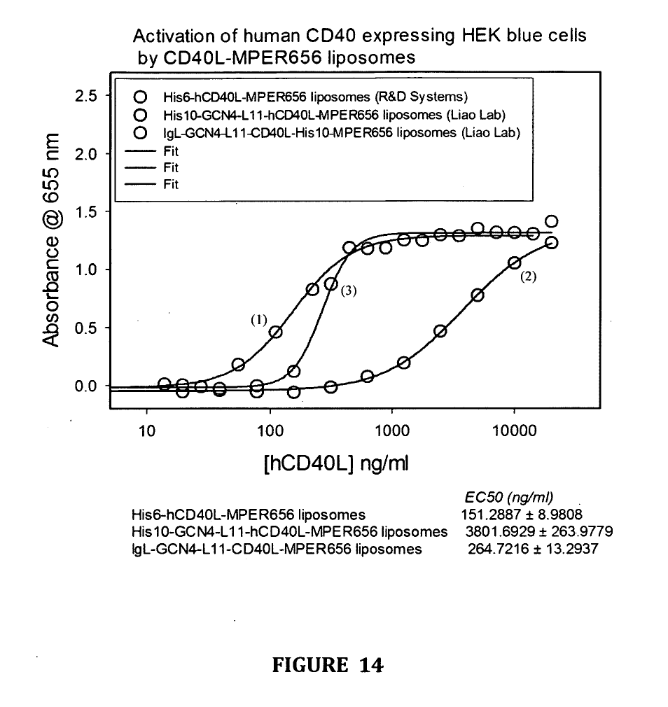

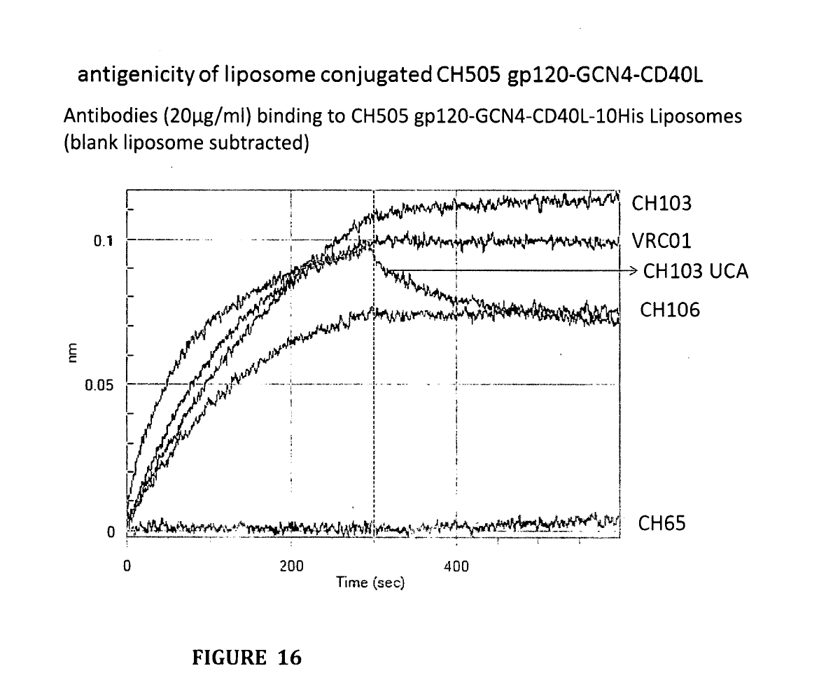

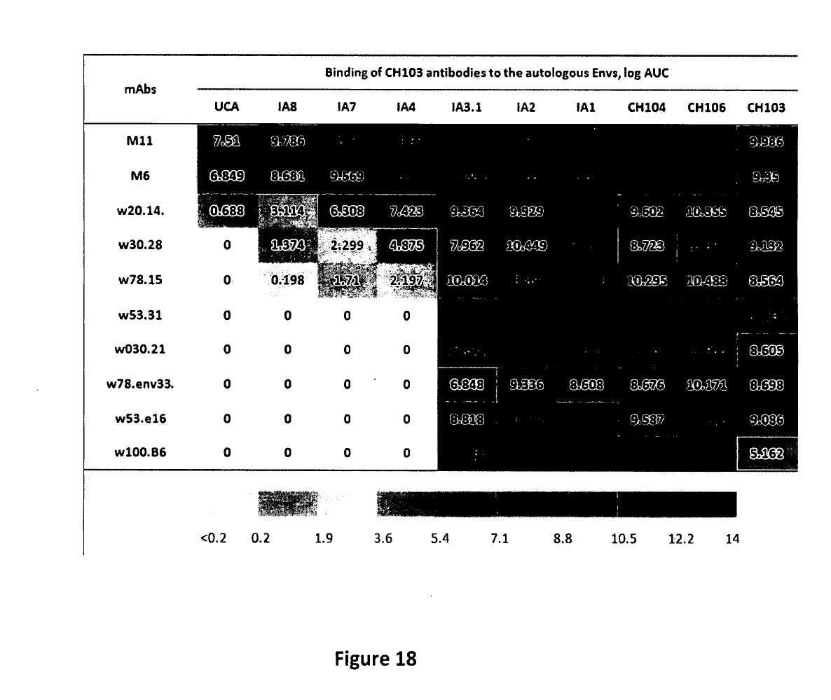

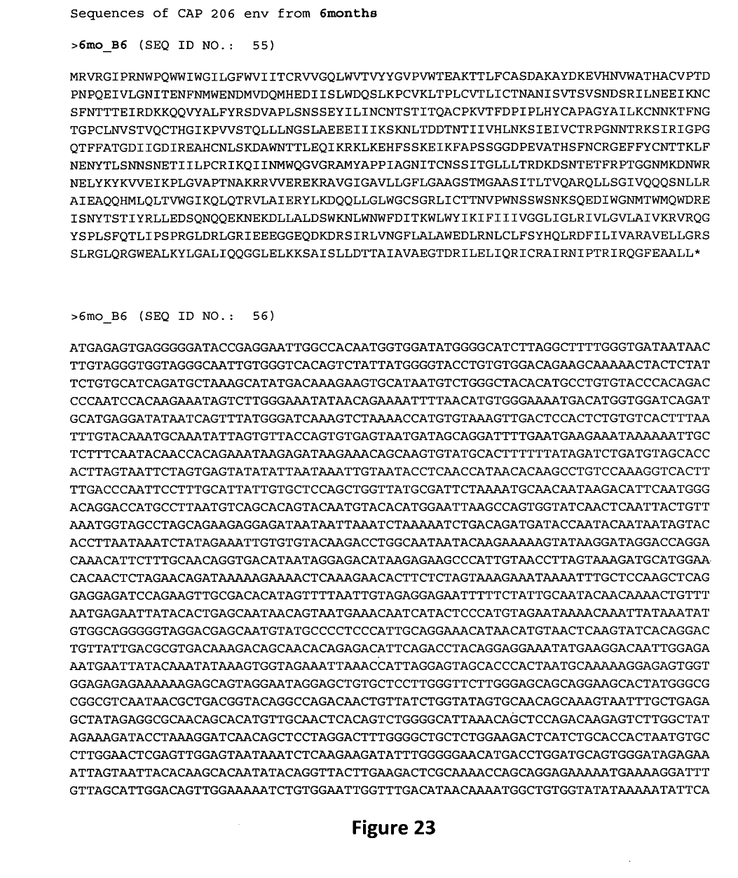

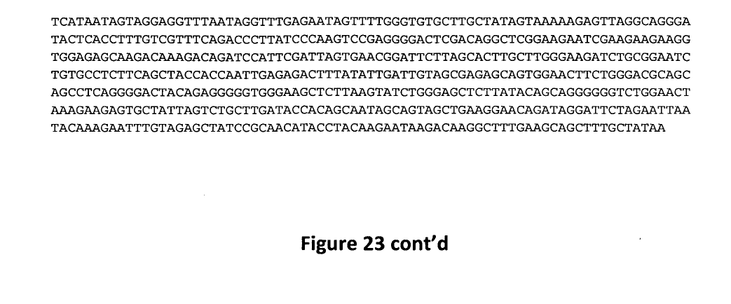

Using the instant disclosure of envelope timers, any HIV-1 envelope can be designed as a trimer. In certain embodiments the HIV-1 envelope is any one of the envelopes or selection of envelopes in Application WO2014042669 (PCT/US PCT/US2013/000210), U.S. Application Ser. No. 61/955,402 (“Swarm Immunization with Envelopes form CH505” Examples 2-4, FIGS. 14-19); U.S. Application Ser. Nos. 61/972,531 and 62/027,427 (Examples 2-3, FIGS. 18-19, 20A-20D, 21A-21C, and 22-24) the contents of which applications are herein incorporated by reference in their entirety.

In certain embodiments, the compositions and methods include any immunogenic HIV-1 sequences to give the best coverage for T cell help and cytotoxic T cell induction. In certain embodiments, the compositions and methods include mosaic and/or consensus HIV-1 genes to give the best coverage for T cell help and cytotoxic T cell induction. In certain embodiments, the compositions and methods include mosaic group M and/or consensus genes to give the best coverage for T cell help and cytotoxic T cell induction. In some embodiments, the mosaic genes are any suitable gene from the HIV-1 genome. In some embodiments, the mosaic genes are Env genes, Gag genes, Pol genes, Nef genes, or any combination thereof. See e.g. U.S. Pat. No. 7,951,377. In some embodiments the mosaic genes are bivalent mosaics. In some embodiments the mosaic genes are trivalent. In some embodiments, the mosaic genes are administered in a suitable vector with each immunization with Env gene inserts in a suitable vector and/or as a protein. In some embodiments, the mosaic genes, for example as bivalent mosaic Gag group M consensus genes, are administered in a suitable vector, for example but not limited to HSV2, would be administered with each immunization with Env gene inserts in a suitable vector, for example but not limited to HSV-2.

In certain aspects the invention provides compositions and methods of Env genetic immunization either alone or with Env proteins to recreate the swarms of evolved viruses that have led to bnAb induction. Nucleotide-based vaccines offer a flexible vector format to immunize against virtually any protein antigen. Currently, two types of genetic vaccination are available for testing—DNAs and mRNAs.

In certain aspects the invention contemplates using immunogenic compositions wherein immunogens are delivered as DNA. See Graham B S, Enama M E, Nason M C, Gordon I J, Peel S A, et al. (2013) DNA Vaccine Delivered by a Needle-Free Injection Device Improves Potency of Priming for Antibody and CD8+ T-Cell Responses after rAd5 Boost in a Randomized Clinical Trial. PLoS ONE 8(4): e59340, page 9. Various technologies for delivery of nucleic acids, as DNA and/or RNA, so as to elicit immune response, both T-cell and humoral responses, are known in the art and are under developments. In certain embodiments, DNA can be delivered as naked DNA. In certain embodiments, DNA is formulated for delivery by a gene gun. In certain embodiments, DNA is administered by electroporation, or by a needle-free injection technologies, for example but not limited to Biojector® device. In certain embodiments, the DNA is inserted in vectors. The DNA is delivered using a suitable vector for expression in mammalian cells. In certain embodiments the nucleic acids encoding the envelopes are optimized for expression. In certain embodiments DNA is optimized, e.g. codon optimized, for expression. In certain embodiments the nucleic acids are optimized for expression in vectors and/or in mammalian cells. In non-limiting embodiments these are bacterially derived vectors, adenovirus based vectors, rAdenovirus (Barouch D H, et al. Nature Med. 16: 319-23, 2010), recombinant mycobacteria (i.e., rBCG or M smegmatis) (Yu, J S et al. Clinical Vaccine Immunol. 14: 886-093, 2007; ibid 13: 1204-11, 2006), and recombinant vaccinia type of vectors (Santra S. Nature Med. 16: 324-8, 2010), for example but not limited to ALVAC, replicating (Kibler K V et al., PLoS One 6: e25674, 2011 nov 9.) and non-replicating (Perreau M et al. J. virology 85: 9854-62, 2011) NYVAC, modified vaccinia Ankara (MVA)), adeno-associated virus, Venezuelan equine encephalitis (VEE) replicons, Herpes Simplex Virus vectors, and other suitable vectors.

In certain aspects the invention contemplates using immunogenic compositions wherein immunogens are delivered as DNA or RNA in suitable formulations. Various technologies which contemplate using DNA or RNA, or may use complexes of nucleic acid molecules and other entities to be used in immunization. In certain embodiments, DNA or RNA is administered as nanoparticles consisting of low dose antigen-encoding DNA formulated with a block copolymer (amphiphilic block copolymer 704). See Cany et al., Journal of Hepatology 2011 vol. 54 j 115-121; Arnaoty et al., Chapter 17 in Yves Bigot (ed.), Mobile Genetic Elements: Protocols and Genomic pplications, Methods in Molecular Biology, vol. 859, pp 293-305 (2012); Arnaoty et al. (2013) Mol Genet Genomics. 2013 August; 288(7-8):347-63. Nanocarrier technologies called Nanotaxi® for immunogenic macromolecules (DNA, RNA, Protein) delivery are under development. See www.incellart.com/en/research-and-development/technologies.html.

In certain aspects the invention contemplates using immunogenic compositions wherein immunogens are delivered as recombinant proteins. Various methods for production and purification of recombinant proteins suitable for use in immunization are known in the art.

The immunogenic envelopes can also be administered as a protein boost in combination with a variety of nucleic acid envelope primes (e.g., HIV-1 Envs delivered as DNA expressed in viral or bacterial vectors).

Dosing of proteins and nucleic acids can be readily determined by a skilled artisan. A single dose of nucleic acid can range from a few nanograms (ng) to a few micrograms (μg) or milligram of a single immunogenic nucleic acid. Recombinant protein dose can range from a few μs micrograms to a few hundred micrograms, or milligrams of a single immunogenic polypeptide.

Administration: The compositions can be formulated with appropriate carriers using known techniques to yield compositions suitable for various routes of administration. In certain embodiments the compositions are delivered via intramascular (IM), via subcutaneous, via intravenous, via nasal, via mucosal routes.

The compositions can be formulated with appropriate carriers and adjuvants using techniques to yield compositions suitable for immunization. The compositions can include an adjuvant, such as, for example but not limited to, alum, poly IC, MF-59 or other squalene-based adjuvant, ASOIB, or other liposomal based adjuvant suitable for protein or nucleic acid immunization. In certain embodiments, TLR agonists are used as adjuvants. In some embodiments, the TLR agonist is a TLR4 agonist, such as but not limited to GLA/SE. In other embodiment, adjuvants which break immune tolerance are included in the immunogenic compositions. In some embodiments the adjuvant is TLR7 or a TLR7/8 agonist, or a TLR-9 agonist, or a combination thereof. See PCT/US2013/029164.

There are various host mechanisms that control bNAbs. For example highly somatically mutated antibodies become autoreactive and/or less fit (Immunity 8: 751, 1998; PloS Comp. Biol. 6 e1000800, 2010; J: Thoret. Biol. 164:37, 1993); Polyreactive/autoreactive naïve B cell receptors (unmutated common ancestors of clonal lineages) can lead to deletion of Ab precursors (Nature 373: 252, 1995; PNAS 107: 181, 2010; J. Immunol. 187: 3785, 2011); Abs with long HCDR3 can be limited by tolerance deletion (JI 162: 6060, 1999; JCI 108: 879, 2001). BnAb knock-in mouse models are providing insights into the various mechanisms of tolerance control of MPER BnAb induction (deletion, anergy, receptor editing). Other variations of tolerance control likely will be operative in limiting BnAbs with long HCDR3s, high levels of somatic hypermutations. 2F5 and 4E10 BnAbs were induced in mature antibody knock-in mouse models with MPER peptide-liposome-TLR immunogens. Next step is immunization of germline mouse models and humans with the same immunogens.

In certain embodiments the immunogens and compositions of the invention comprise immunostimulatory components. In a non-limiting embodiment, the immunogen comprises a CD40L.

### EXAMPLES

### Example 1: GCN4 Envelope Trimers and CD40L Containing Immunogens Bind HIV-1 Envelope Antibodies and are Functionally Active

Provided is one example of the design and formulation of liposomes that present immune-modulating CD40 ligand (CD40L) and HIV-1 gp41 neutralizing antigen. CD40L, the ligand for CD40 expressed on B-cell surface is anchored on the liposomes that had HIV-1 gp41 MPER peptide immunogen conjugated in them. Two broadly neutralizing gp41 membrane proximal external region (MPER) antibodies (2F5, 4E10) bound strongly to CD40L conjugated MPER peptide liposomes. This construct has important application as an experimental AIDS vaccine in providing immune-modulating effect to stimulate proliferation of B-cells capable of producing neutralizing antibodies targeting HIV-1 gp41 MPER region.

CD40L-Gp41 MPER Peptide-Liposome Conjugates:

Recombinant CD40L with an N-terminal Histidine Tag (MGSSHHHHHH SSGLVPRGSH MQKGDQNPQI AAHVISEASS KTTSVLQWAE KGYYTMSNNL VTLENGKQLT VKRQGLYYIY AQVTFCSNRE ASSQAPFIAS LCLKSPGRFE RILLRAANTH SSAKPCGQQS IHLGGVFELQ PGASVFVNVT DPSQVSHGTG FTSFGLLKL) was anchored to MPER peptide liposomes via His-Ni-NTA chelation by mixing CD40L with MPER656-Ni-NTA liposomes at 1:50 CD40L and Ni-NTA molar ratio (FIG. 12).

The construction of MPER peptide Ni-NTA liposomes utilized the method of co-solubilization of MPER peptide having a membrane anchoring amino acid sequence and synthetic lipids 1-Palmitoyl-2-Oleoyl-sn-Glycero-3-Phosphocholine (POPC), 1-Palmitoyl-2-Oleoyl-sn-Glycero-3-Phosphoethanolamine (POPE), 1,2-Dimyristoyl-sn-Glycero-3-Phosphate (DMPA), Cholesterol and 1,2-dioleoyl-sn-Glycero-3-[(N-(5-amino-1-carboxypentyl)iminodiacetic acid)succinyl] (nickel salt) (DGS-NTA(Ni) at mole fractions 0.216, 35.00, 25.00, 20.00, 1.33 and 10 respectively. Appropriate amount of MPER peptide dissolved in chloroform-methanol mixture (7:3 v/v), appropriate amounts of chloroform stocks of phospholipids were dried in a stream of nitrogen followed by over night vacuum drying. Liposomes were made from the dried peptide-lipid film in phosphate buffered saline (pH 7.4) using extrusion technology.

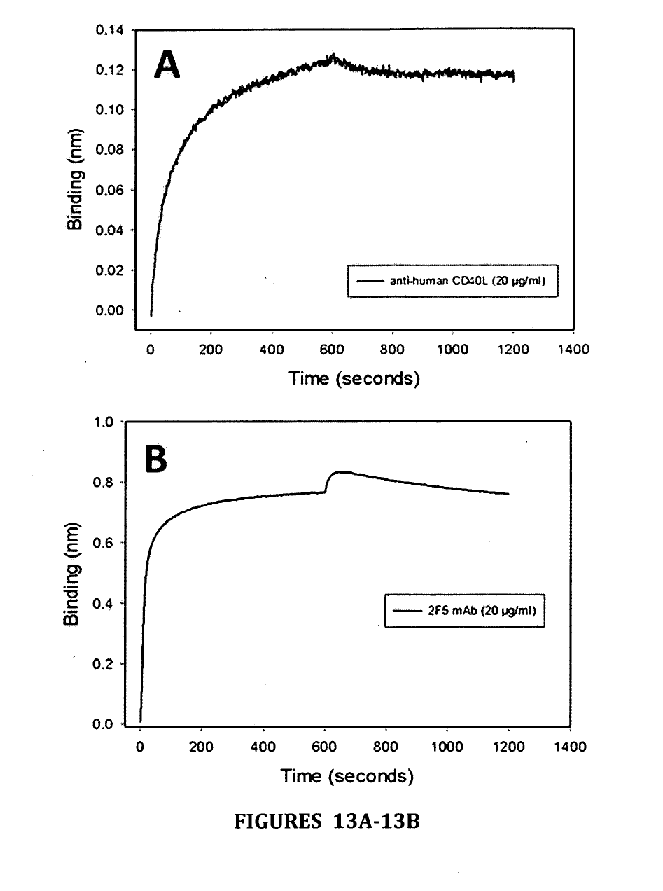

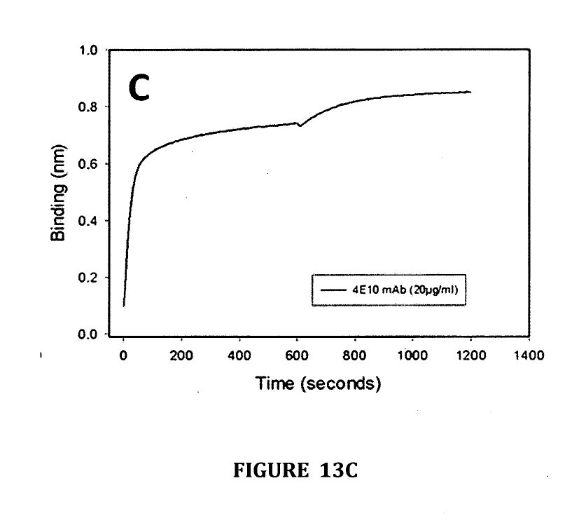

Biolayer interferometry (BLI) assay showed the binding of anti-human CD40L antibody to CD40L-MPER656 liposomes and confirmed the correct presentation of CD40 L on liposome surface (FIG. 13A). The broadly neutralizing HIV-1 gp41 MPER antibodies 2F5 and 4E10 bound strongly to CD40L-MPER656 liposomes (FIGS. 13B-13C) and demonstrated that the CD40L co-display did not impede the presentation of the epitopes of 2F5 and 4E10 mAbs.

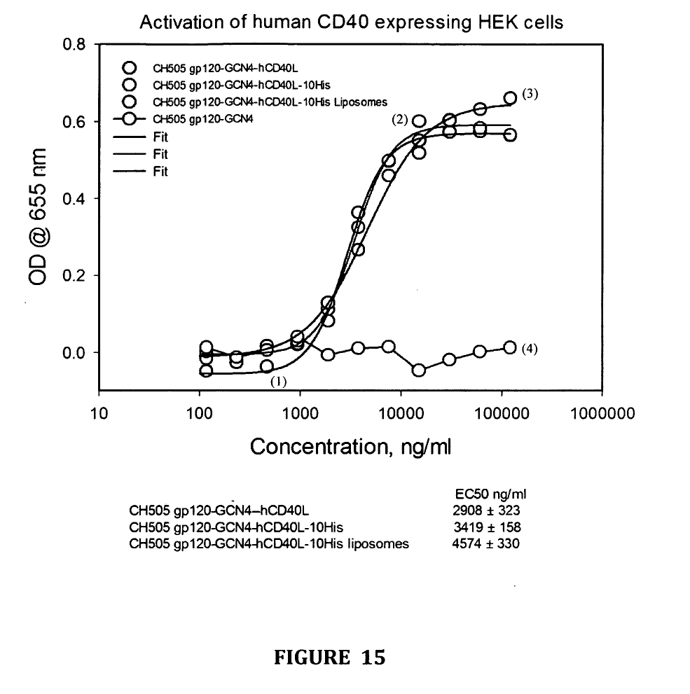

FIGS. 14 and 15 show CD40L containing immunogens activate human CD40 expressing HEK cells.

### Example 2—Combination of Antigens from CH505 Envelope Sequences for Immunization

Provided herein are non-limiting examples of combinations of antigens derived from CH505 envelope sequences for a swarm immunization. The selection includes priming with a virus which binds to the UCA, for example a T/F virus or another early (e.g. but not limited to week 004.3, or 004.26) virus envelope. In certain embodiments the prime could include D-loop variants. In certain embodiments the boost could include D-loop variants.

Non-limiting embodiments of envelopes selected for swarm vaccination are shown as the selections described below. A skilled artisan would appreciate that a vaccination protocol can include a sequential immunization starting with the “prime” envelope(s) and followed by sequential boosts, which include individual envelopes or combination of envelopes. In another vaccination protocol, the sequential immunization starts with the “prime” envelope(s) and is followed with boosts of cumulative prime and/or boost envelopes. In certain embodiments, the prime does not include T/F sequence (W000.TF). In certain embodiments, the prime includes w004.03 envelope. In certain embodiments, the prime includes w004.26 envelope. In certain embodiments, the immunization methods do not include immunization with HIV-1 envelope T/F. In other embodiments for example the T/F envelope may not be included when w004.03 or w004.26 envelope is included. In certain embodiments, there is some variance in the immunization regimen; in some embodiments, the selection of HIV-1 envelopes may be grouped in various combinations of primes and boosts, either as nucleic acids, proteins, or combinations thereof.

In certain embodiments the immunization includes a prime administered as DNA, and MVA boosts. See Goepfert, et al. 2014; “Specificity and 6-Month Durability of Immune Responses Induced by DNA and Recombinant Modified Vaccinia Ankara Vaccines Expressing HIV-1 Virus-Like Particles” J Infect Dis. 2014 Feb. 9. [Epub ahead of print].

HIV-1 Envelope selection A (ten envelopes sensitive envelopes): 703010505.TF, 703010505.W4.03, 703010505.W4.26, 703010505.W14.21, 703010505.W20.14, 703010505.W30.28, 703010505.W30.13, 703010505.W53.31, 703010505.W78.15, 703010505.W100.B4, optionally in certain embodiments designed as trimers. See U.S. Provisional Application No. 62/027,427 incorporated by reference.

HIV-1 Envelope selection B (twenty envelopes sensitive envelopes): 703010505.TF, 703010505.W4.03, 703010505.W4.26, 703010505.W14.3, 703010505.W14.8, 703010505.W14.21, 703010505.W20.7, 703010505.W20.26, 703010505.W20.9, 703010505.W20.14, 703010505.W30.28, 703010505.W30.12, 703010505.W30.19, 703010505.W30.13, 703010505.W53.19, 703010505.W53.13, 703010505.W53.31, 703010505.W78.1, 703010505.W78.15, 703010505.W100.B4, optionally in certain embodiments designed as trimers. See U.S. Provisional Application No. 62/027,427 incorporated by reference.

HIV-1 Envelope selection C (four envelopes): 703010505.TF, 703010505.W53.16, 703010505.W78. 33, 703010505.W100.B6, optionally in certain embodiments designed as trimers. See WO2014042669, the contents of which are hereby incorporated by reference.

HIV-1 Envelope selection D (ten production envelopes): CH505.M6D8gp120;

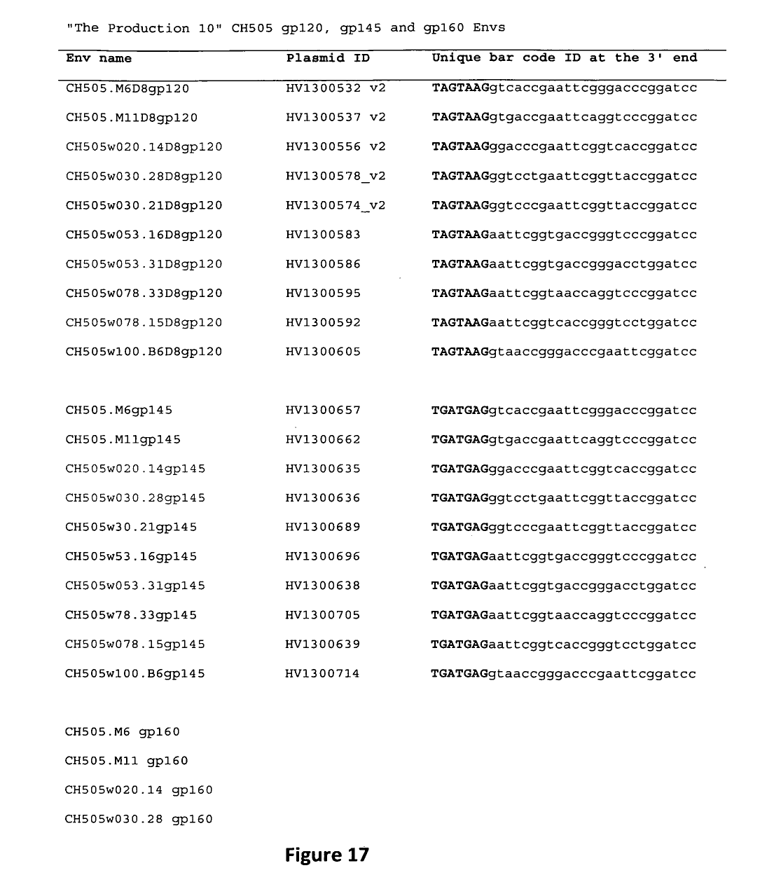

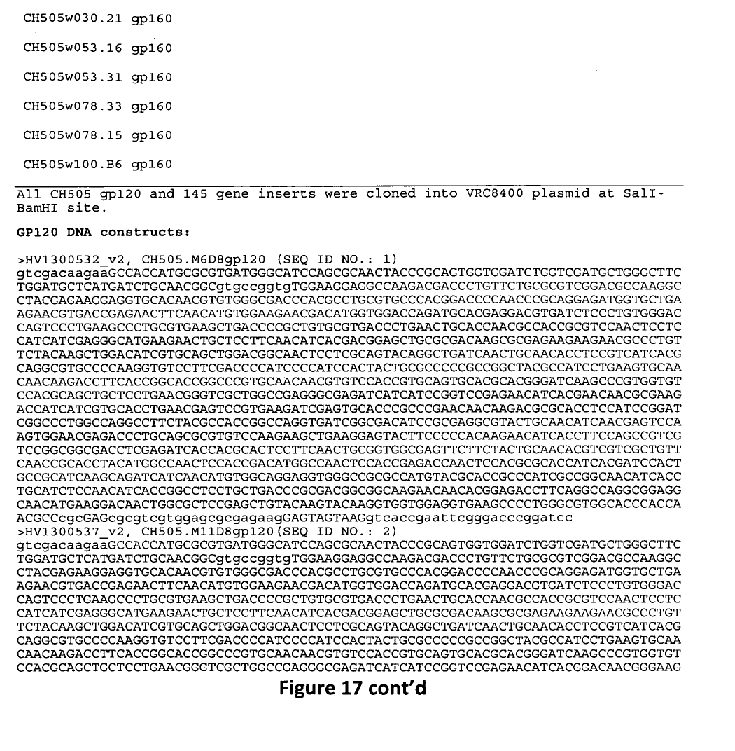

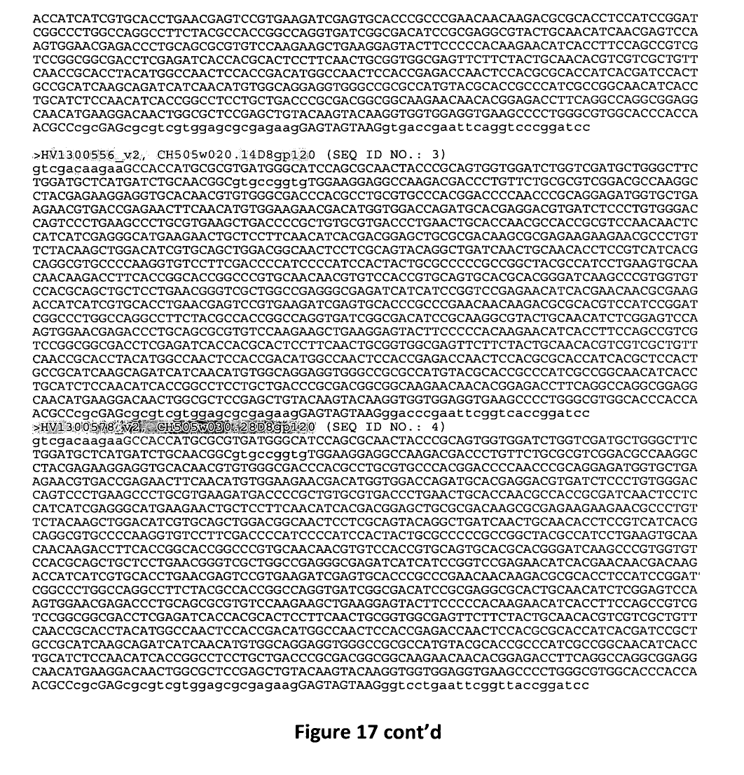

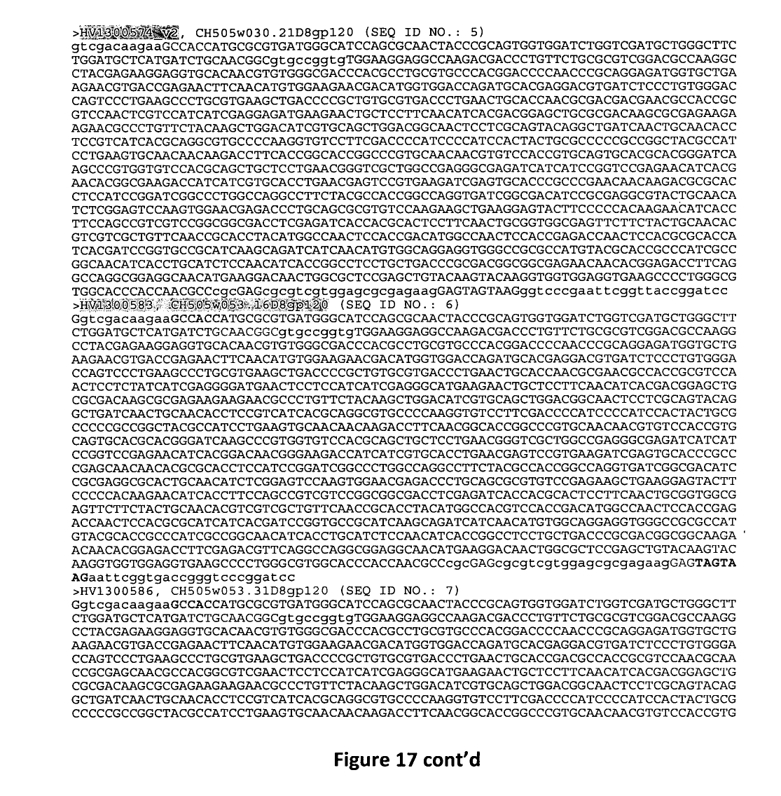

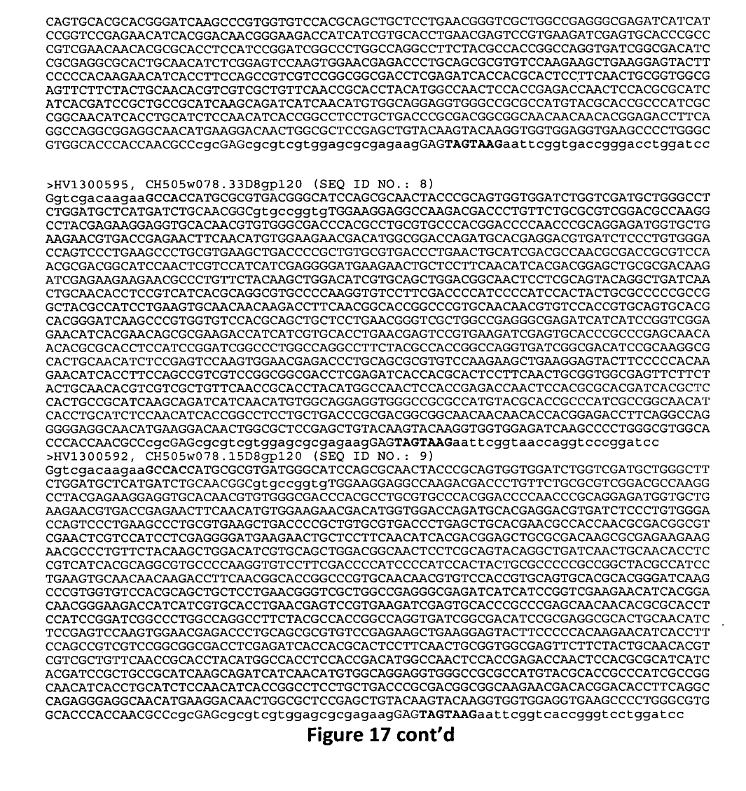

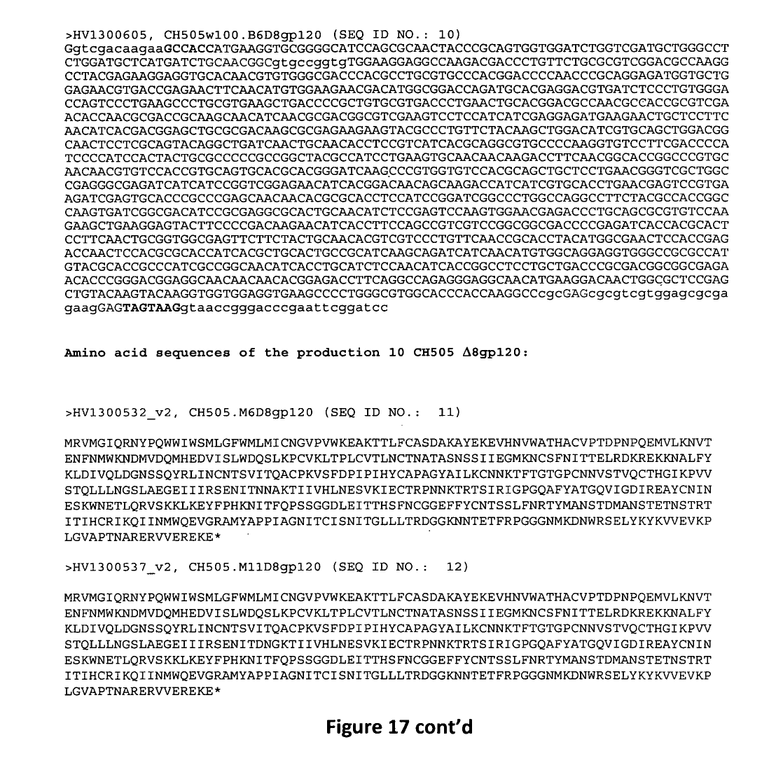

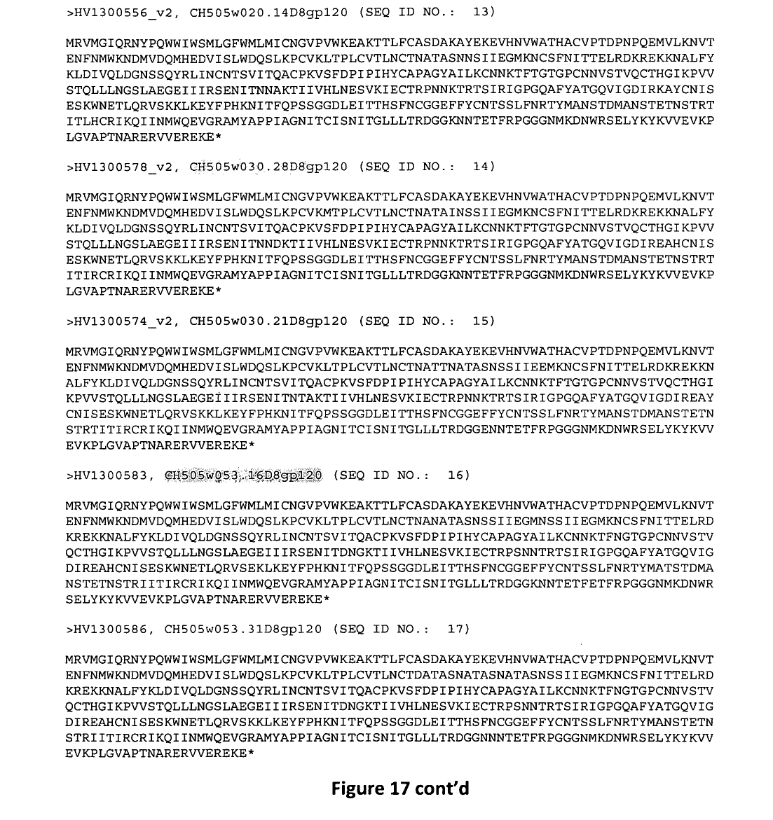

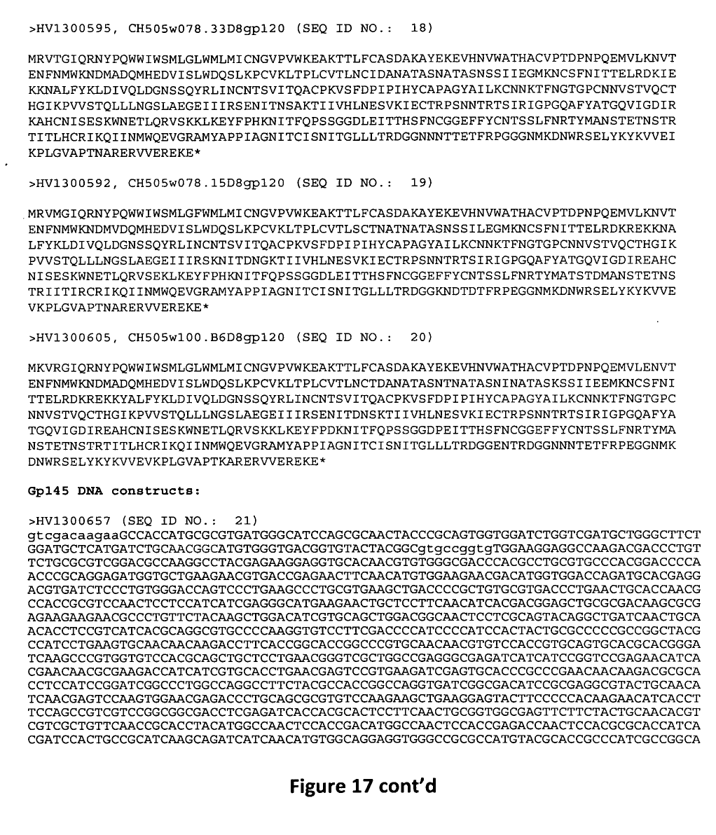

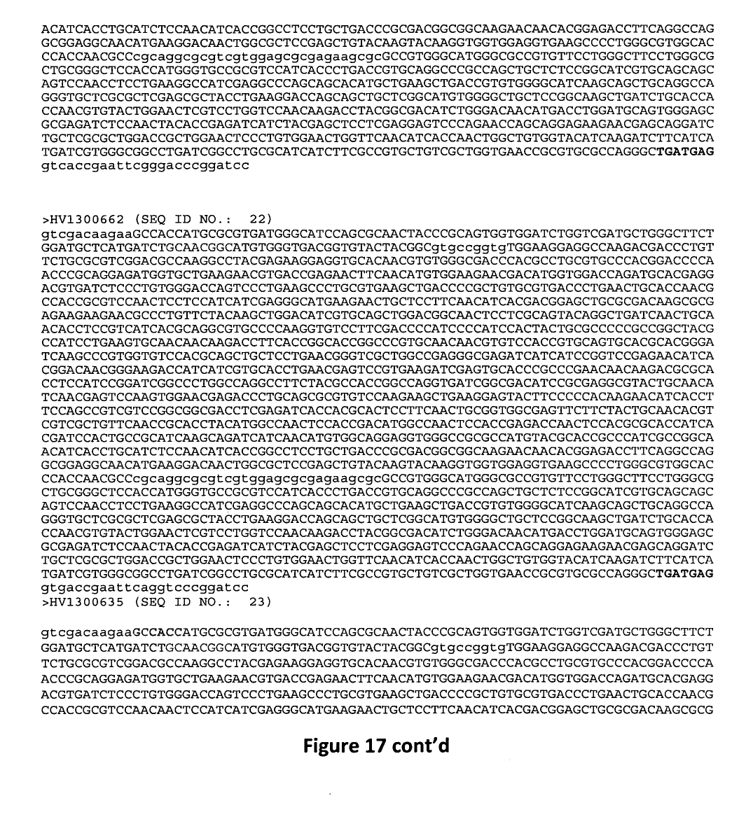

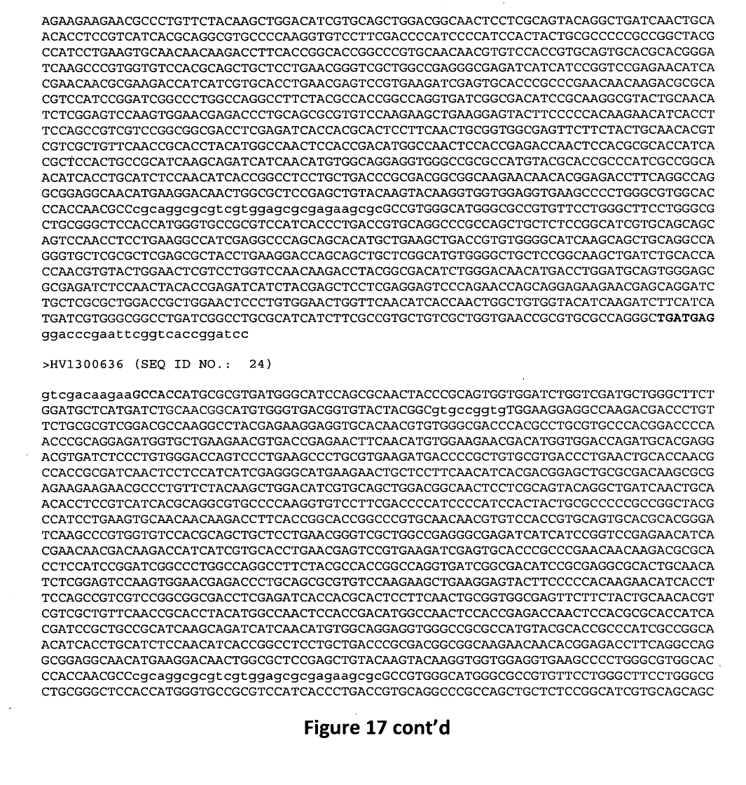

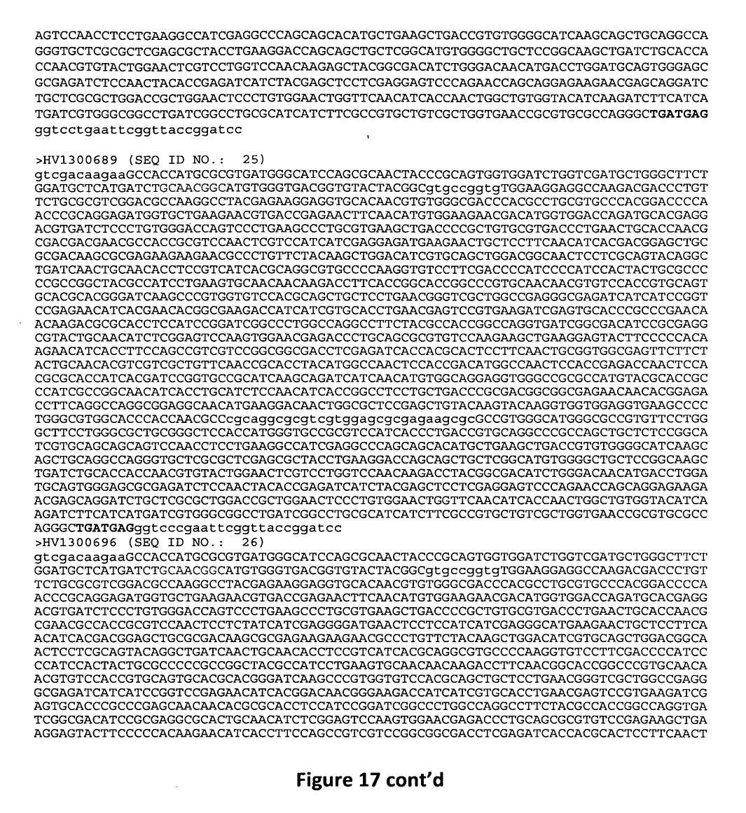

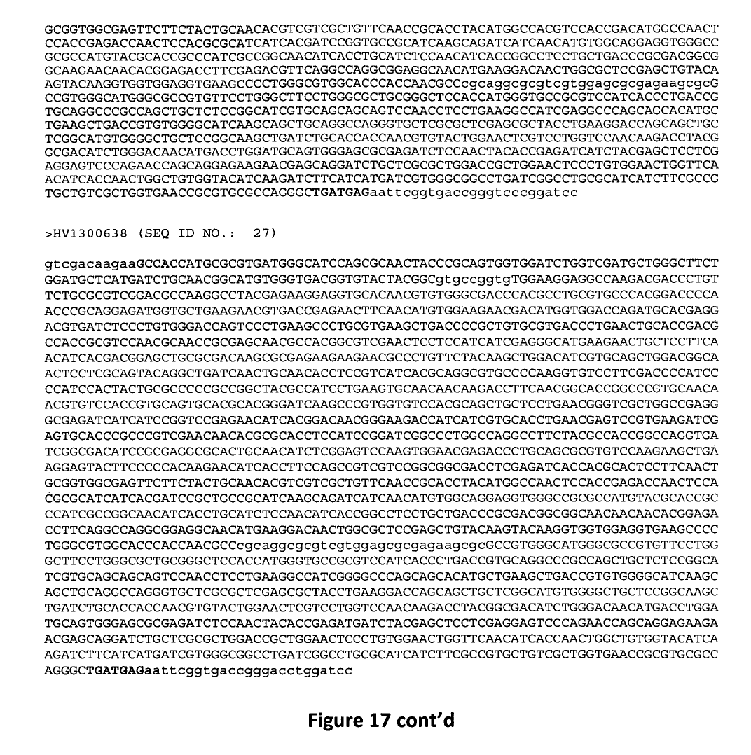

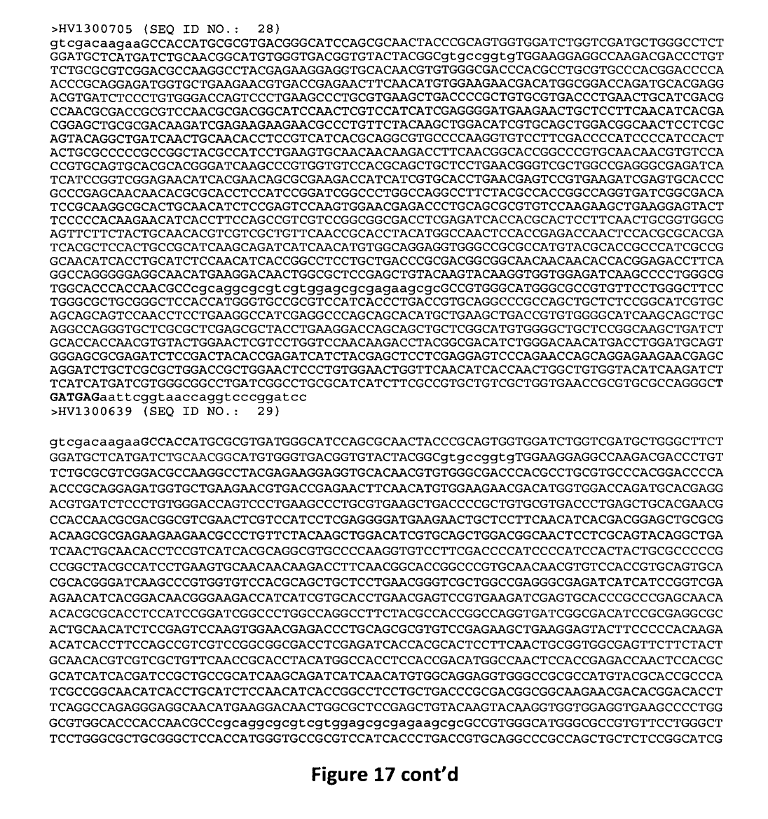

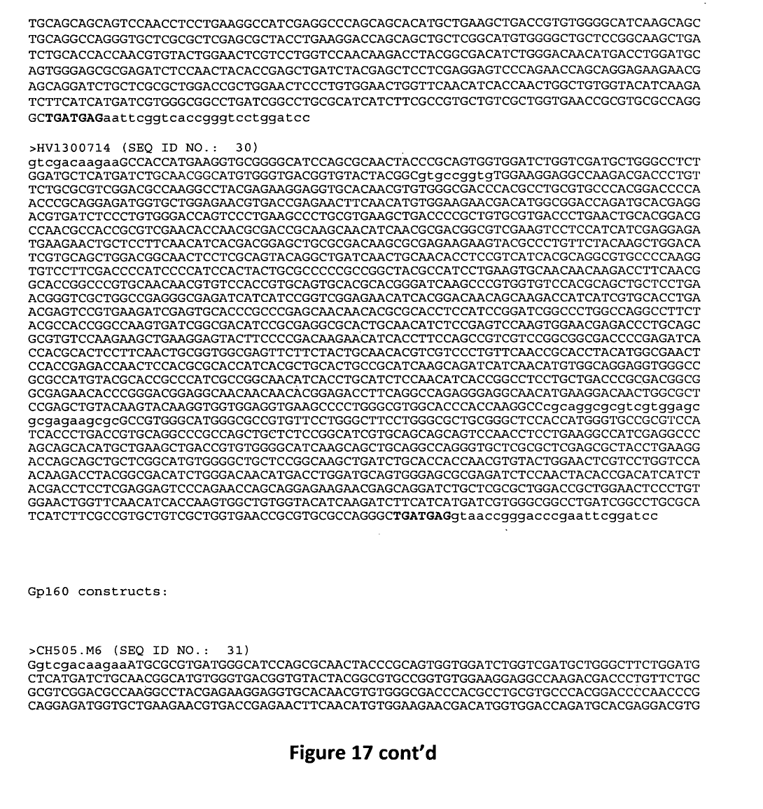

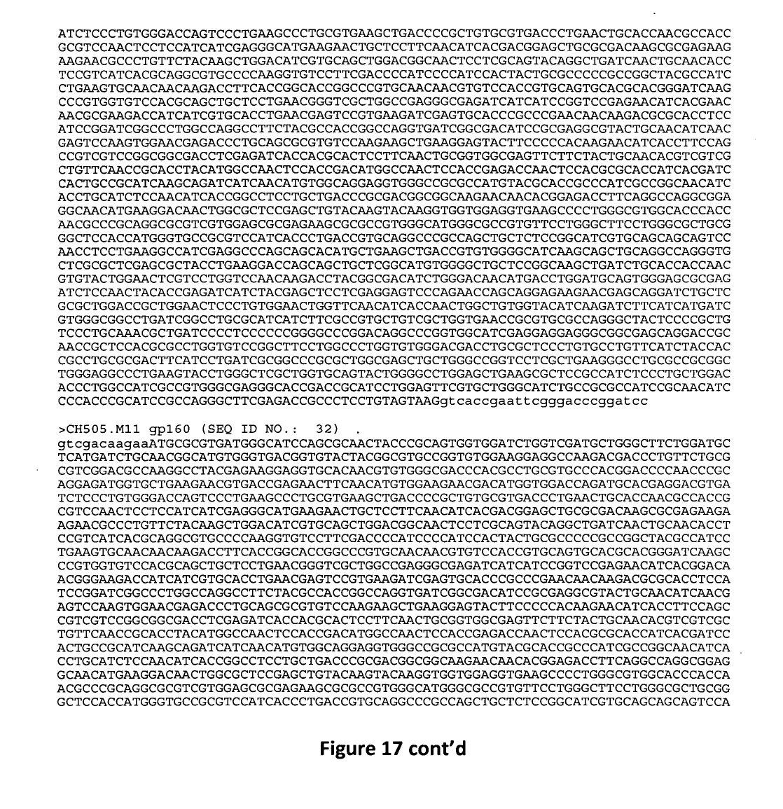

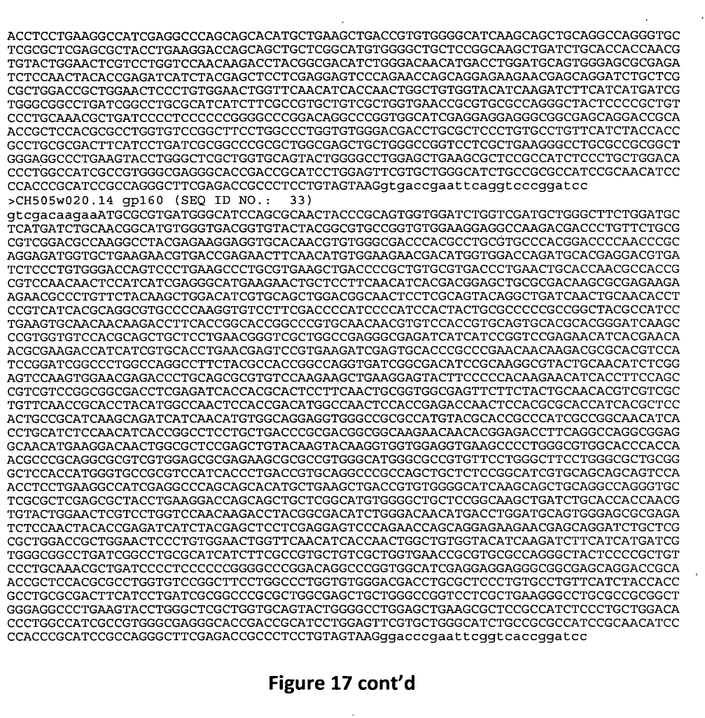

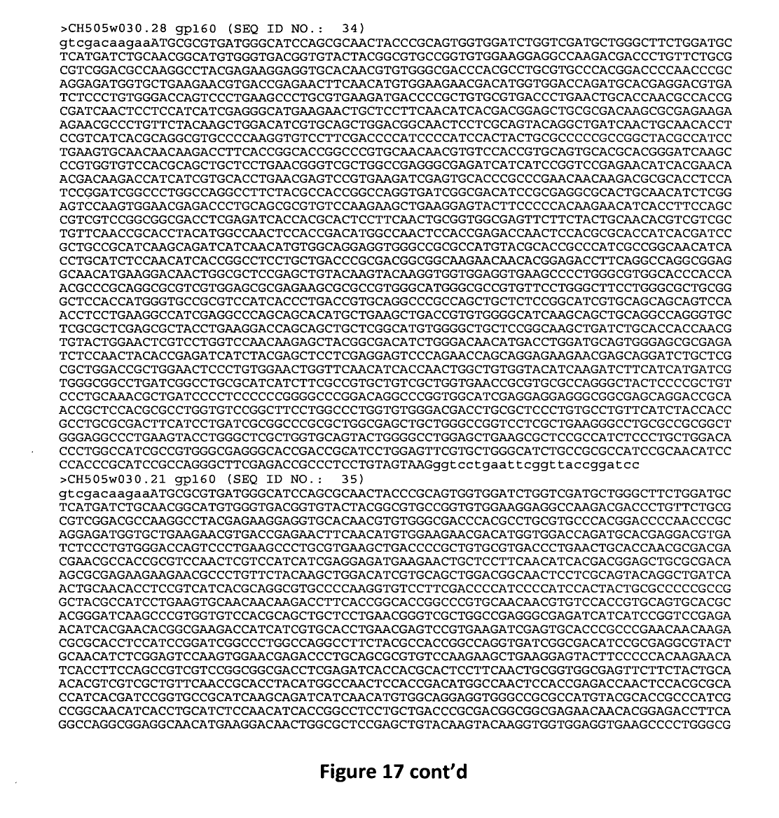

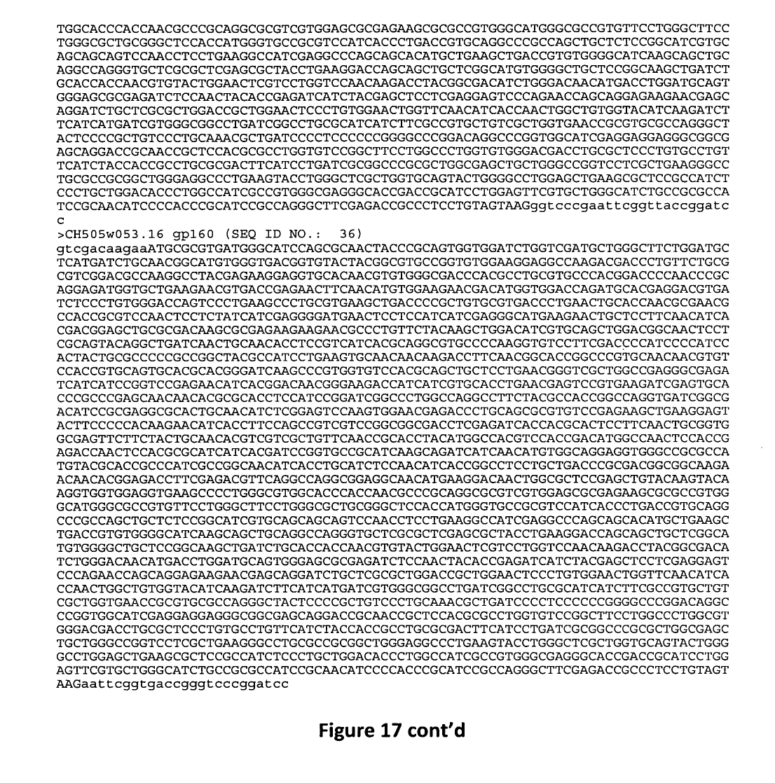

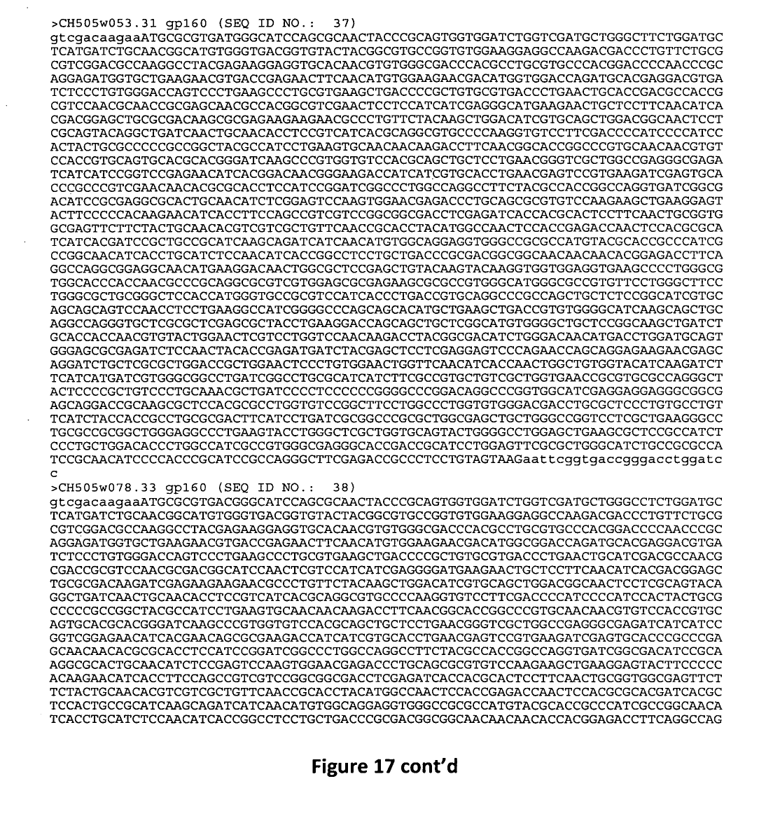

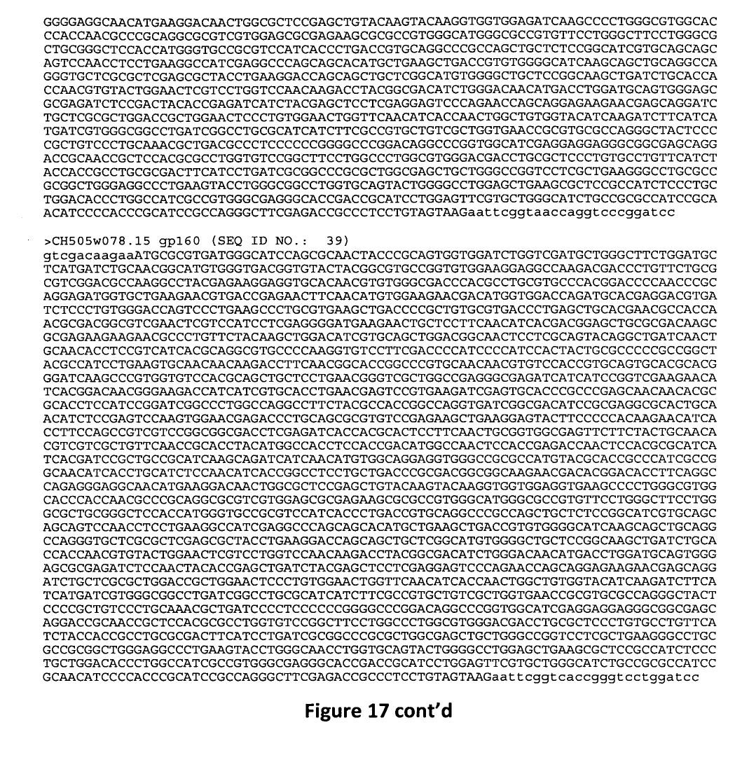

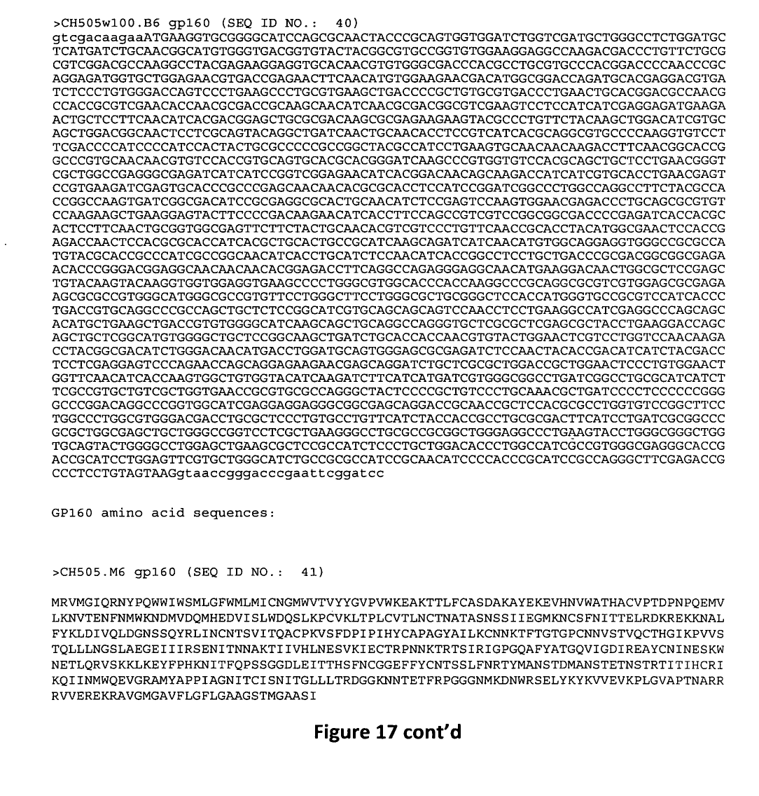

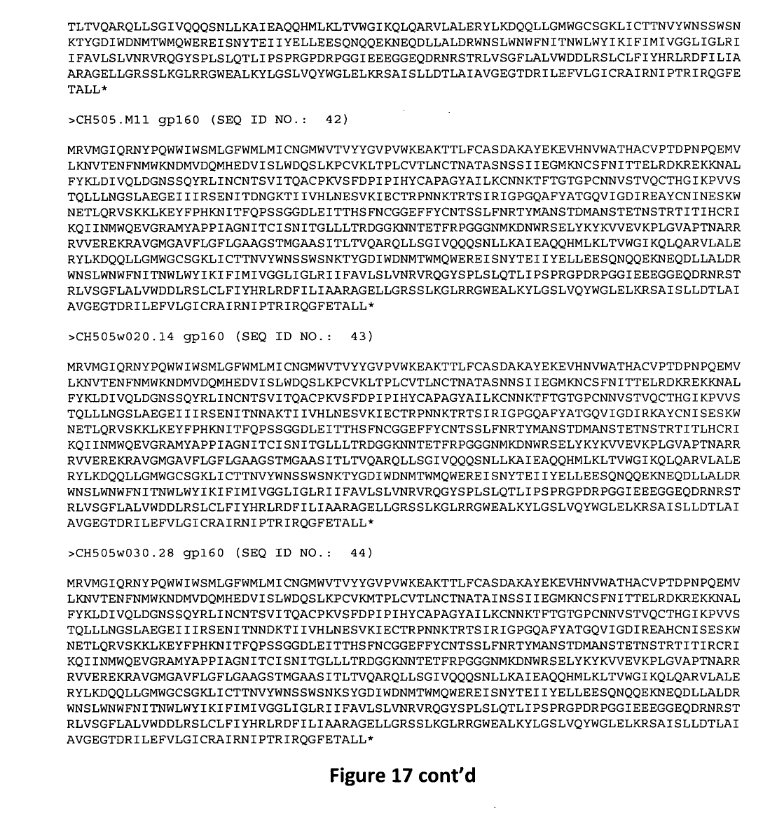

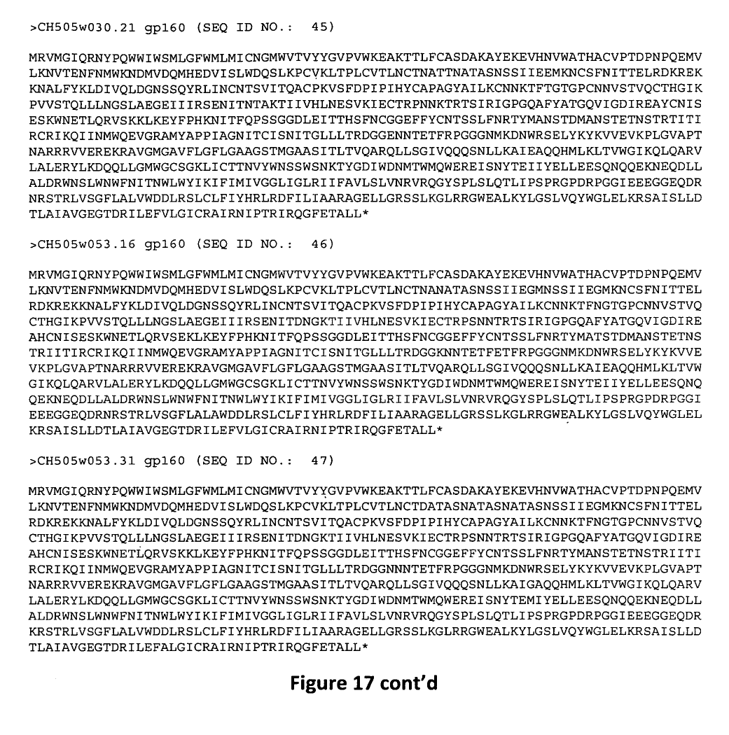

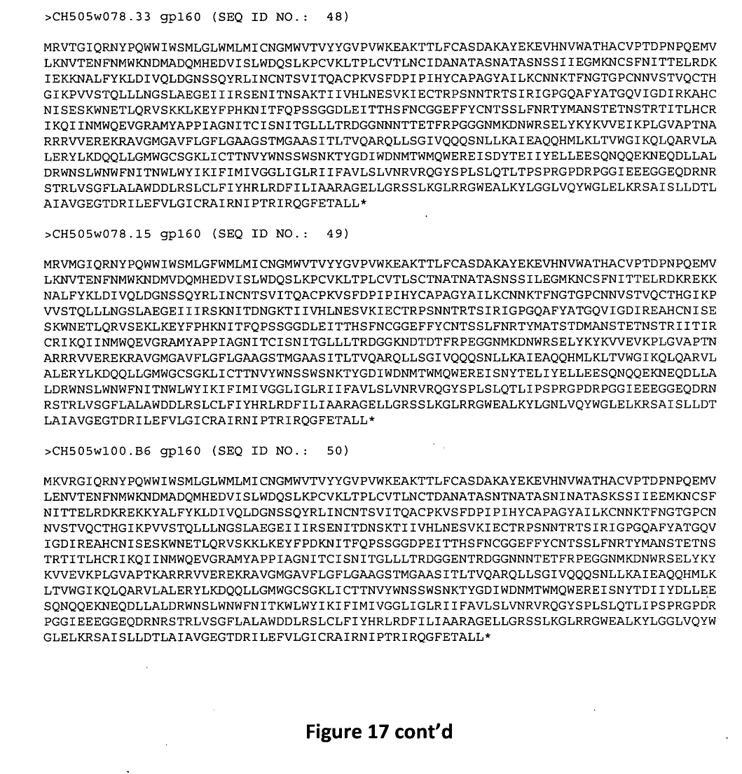

CH505.M11D8gp120; CH505w020.14D8gp120; CH505w030.28D8gp120; CH505w030.21D8gp120; CH505w053.16D8gp120; CH505w053.31D8gp120; CH505w078.33D8gp120; CH505w078.15D8gp120; CH505w100.B6D8gp120, optionally in certain embodiments designed as trimers. See FIG. 17.

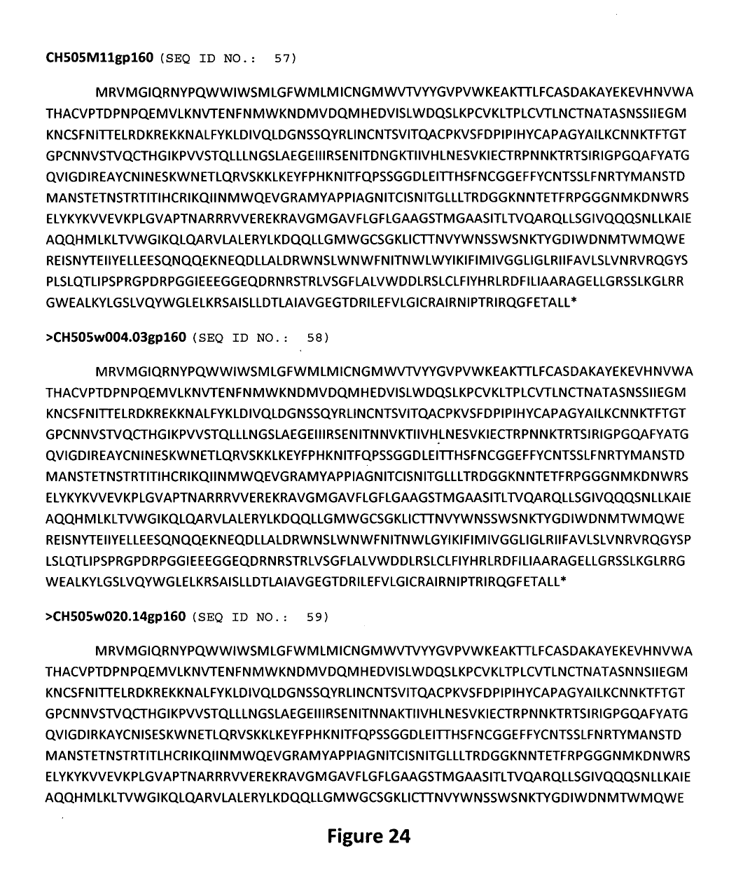

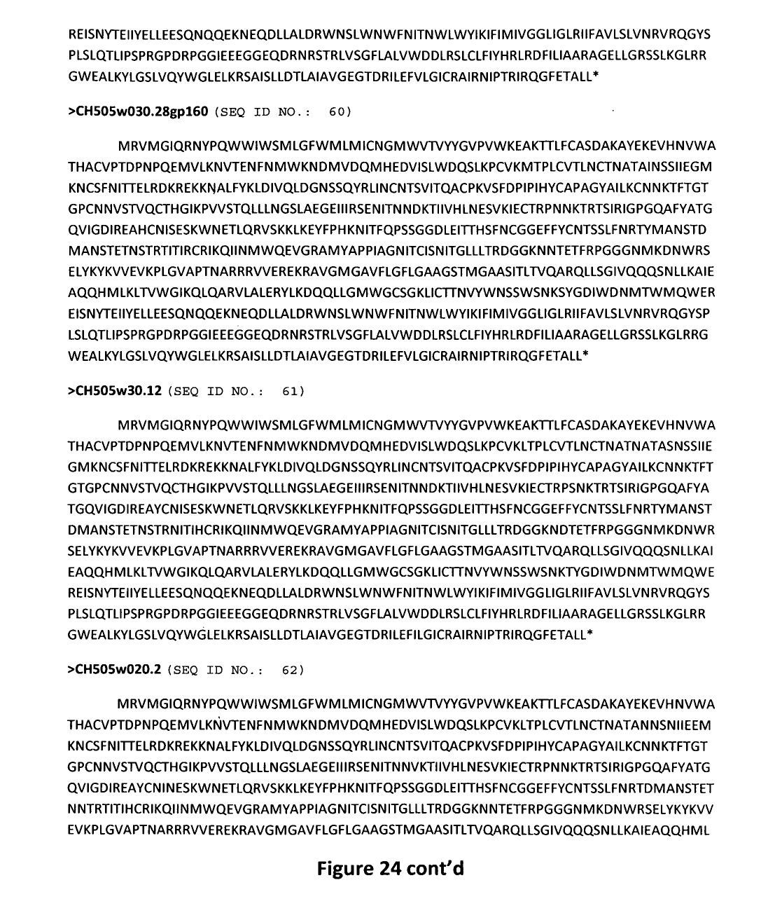

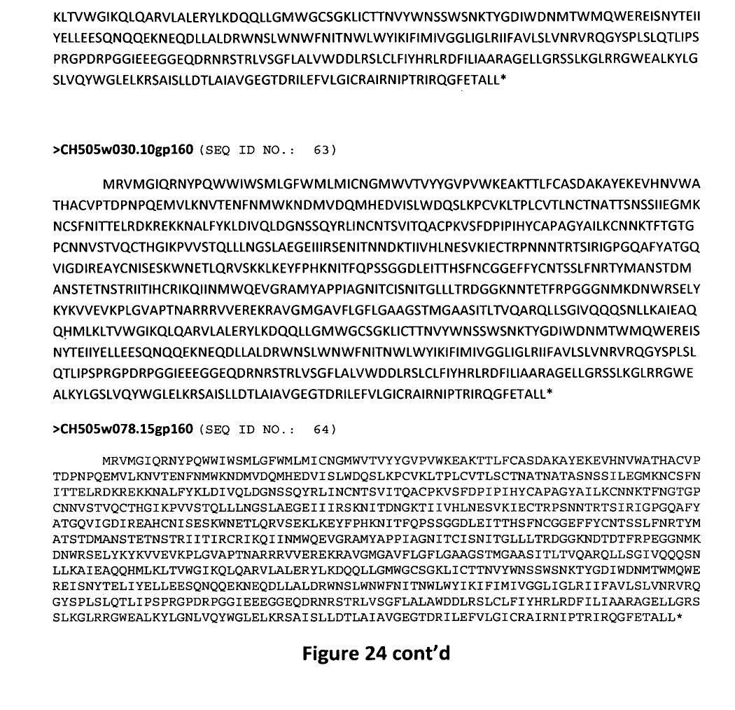

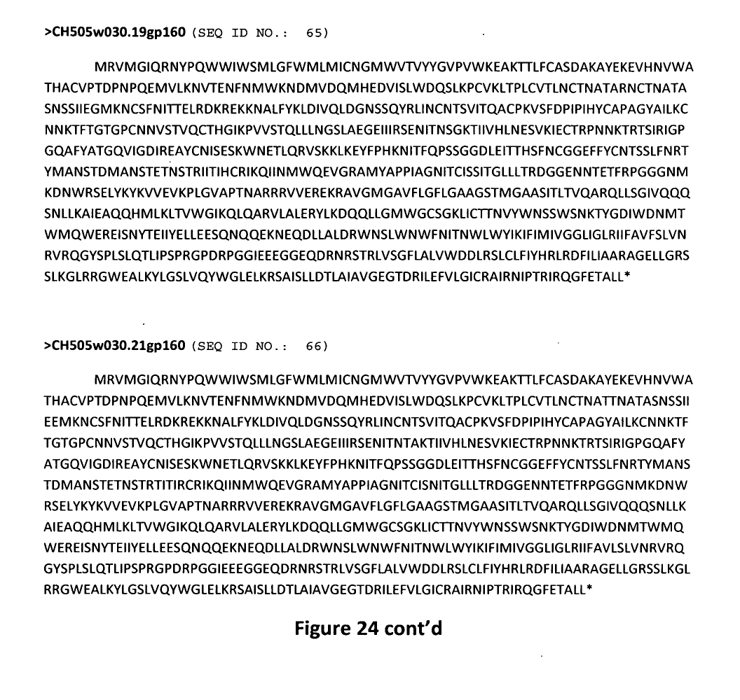

HIV-1 Envelopes selection E (ten early envelopes): optionally in certain embodiments designed as trimers. CH505.M11; CH505.w004.03; CH505.w020.14; CH505.w030.28; CH505.w030.12; CH505.w020.2; CH505.w030.10; CH505.w078.15; CH505.w030.19; CH505.w030.21, optionally in certain embodiments designed as trimers. See FIG. 24.

HIV-1 Envelope selection F (eight envelopes): M11, T/F Env, week 20.14, week 30.28, week 78.15, week 78.33, week 53.16, and week 100.B6 Envs, optionally in certain embodiments designed as trimers.

### Example 3: Examples of Immunization Protocols in Subjects with Swarms of HIV-1 Envelopes

Immunization protocols contemplated by the invention include envelopes sequences as described herein including but not limited to nucleic acids and/or amino acid sequences of gp160s, gp150s, gp145s, cleaved and uncleaved gp140s, gp120s, gp41s, N-terminal deletion variants as described herein, cleavage resistant variants as described herein, or codon optimized sequences thereof. A skilled artisan can readily modify the gp160 and gp120 sequences described herein to obtain these envelope variants. The swarm immunization protocols can be administered in any subject, for example monkeys, mice, guinea pigs, or human subjects.

In non-limiting embodiments, the immunization includes a nucleic acid is administered as DNA, for example in a modified vaccinia vector (MVA). In non-limiting embodiments, the nucleic acids encode gp160 envelopes. In other embodiments, the nucleic acids encode gp120 envelopes. In other embodiments, the boost comprises a recombinant gp120 envelope. The vaccination protocols include envelopes formulated in a suitable carrier and/or adjuvant, for example but not limited to alum. In certain embodiments the immunizations include a prime, as a nucleic acid or a recombinant protein, followed by a boost, as a nucleic acid or a recombinant protein. A skilled artisan can readily determine the number of boosts and intervals between boosts.

In non-limiting embodiments, the prime includes a 703010505.TF envelope and a loop D variant as described herein. In non-limiting embodiments, the prime includes a 703010505.TF envelope and/or 703010505.W4.03, 703010505.W4.26 envelope, and a loop D variant as described herein. In certain embodiments, the loop D variant is M6. In certain embodiments, the loop D variant is M5. In certain embodiments, the loop D variant is M10. In certain embodiments, the loop D variant is M19. In certain embodiments, the loop D variant is M11. In certain embodiments, the loop D variant is M20. In certain embodiments, the loop D variant is M21. In certain embodiments, the loop D variant is M9. In certain embodiments, the loop D variant is M8. In certain embodiments, the loop D variant is M7.

Table 1 shows a non-limiting example of a sequential immunization protocol using a swarm of HIV1 envelopes (703010505.TF, 703010505.W4.03, 703010505.W4.26, 703010505.W14.21, 703010505.W20.14, 703010505.W30.28, 703010505.W30.13, 703010505.W53.31, 703010505.W78.15, 703010505.W100.B4, optionally in certain embodiments designed as trimers. In a non-limiting embodiment, a suggested grouping for prime and boost is to begin with the CH505 TF+W4.03, then boost with a mixture of w4.26+14.21+20.14, then boost with a mixture of w30.28+30.13+53.31, then boost with a mixture of w78.15+100.B4.

| Envelope | Prime | Boost(s) | Boost(s) | Boost(s) |
|---|---|---|---|---|
| CH505 TF + | CH505 TF + | | | |
| W4.03 | W4.03 as a | | | |
| | nucleic acid e.g. | | | |
| | DNA/MVA | | | |
| | vector and/or | | | |
| | protein | | | |
| w4.26 + | | w4.26 + 14.21 + | | |
| 14.21 + | | 20.14 as a | | |
| 20.14 | | nucleic acid e.g. | | |
| | | DNA/MVA | | |
| | | vector and/or | | |
| | | protein | | |
| w30.28 + | | | w30.28 + | |
| 30.13 + | | | 30.13 + | |
| 53.31 | | | 53.31 as a | |
| | | | nucleic acid | |
| | | | e.g. | |
| | | | DNA/MVA | |
| | | | vector | |
| | | | and/or | |
| | | | protein | |
| w78.15 + | | | | w78.15 + |
| 100.B4 | | | | 100.B4 as |
| | | | | a nucleic |
| | | | | acid e.g. |
| | | | | DNA/MVA |
| | | | | vector |
| | | | | and/or |
| | | | | protein |

A skilled artisan can readily determine the number and interval between boosts.

Table 2 shows a non-limiting example of a sequential immunization protocol using a swarm of HIV1 envelopes optionally in certain embodiments designed as trimers.

| Envelope | Prime | Boost(s) |
|---|---|---|
| 703010505.TF, | 703010505.TF (optionally | 703010505.TF, |
| 703010505.W4.03, | 703010505.W4.03, | 703010505.W4.03, |
| 703010505.W4.26, | 703010505.W4.26) as a | 703010505.W4.26, |
| 703010505.W14.21, | nucleic acid e.g. | 703010505.W14.21, |
| 703010505.W20.14, | DNA/MVA vector | 703010505.W20.14, |
| 703010505.W30.28, | and/or protein | 703010505.W30.28, |
| 703010505.W30.13, | | 703010505.W30.13, |
| 703010505.W53.31, | | 703010505.W53.31, |
| 703010505.W78.15, | | 703010505.W78.15, |
| 703010595.W100.B4. | | 703010505.W100.B4 |
| | | as a nucleic acid |
| | | e.g. DNA/MVA |
| | | vector and/or protein |

A skilled artisan can readily determine the number and interval between boosts

For a 20mer immunization regimen (envelopes (703010505.TF, 703010505.W4.03, 703010505.W4.26, 703010505.W14.3, 703010505.W14.8, 703010505.W14.21, 703010505.W20.7, 703010505.W20.26, 703010505.W20.9, 703010505.W20.14, 703010505.W30.28, 703010505.W30.12, 703010505.W30.19, 703010505.W30.13, 703010505.W53.19, 703010505.W53.13, 703010505.W53.31, 703010505.W78.1, 703010505.W78.15, 703010505.W100.B4), in a non-limiting embodiment, one can prime with CH505 TF+W4.03, then boost with a mixture of w4.26+14.21+20.14+14.3+14.8+20.7, then boost with a mixture of w 20.26+20.9+30.12+w30.28+30.13+53.31, then boost with a mixture of w78.15+100.B4+30.19+53.19+53.13+78.1. Other combinations of envelopes are contemplated for boosts.

Table 3 shows a non-limiting example of a sequential immunization protocol using a swarm of HIV1 envelopes optionally in certain embodiments designed as trimers

| Envelope | Prime | Boost(s) |
|---|---|---|
| 703010505.TF, | 703010505.TF, (optionally | 703010505.TF, |
| 703010505.W4.03, | 703010505.W4.03, | 703010505.W4.03, |
| 703010505.W4.26, | 703010505.W4.26, | 703010505.W4.26, |
| 703010505.W14.3, | 703010505.W14.3, | 703010505.W14.3, |
| 703010505.W14.8, | 703010505.W14.8, | 703010505.W14.8, |
| 703010505.W14.21, | 703010505.W14.21), as a | 703010505.W14.21, |
| 703010505.W20.7, | nucleic acid e.g. | 703010505.W20.7, |
| 703010505.W20.26, | DNA/MVA | 703010505.W20.26, |
| 703010505.W20.9, | vector and/or protein | 703010505.W20.9, |
| 703010505.W20.14, | | 703010505.W20.14, |
| 703010505.W30.28, | | 703010505.W30.28, |
| 703010505.W30.12, | | 703010505.W30.12, |
| 703010505.W30.19, | | 703010505.W30.19, |
| 703010505.W30.13, | | 703010505.W30.13, |
| 703010505.W53.19, | | 703010505.W53.19, |
| 703010505.W53.13, | | 703010505.W53.13, |
| 703010505.W53.31, | | 703010505.W53.31, |
| 703010505.W78.1, | | 703010505.W78.1, |
| 703010505.W78.15, | | 703010505.W78.15, |
| 703010505.W100.B4. | | 703010505.W100.B4. |
| | | as a nucleic acid |
| | | e.g. DNA/MVA |
| | | vector and/or protein |

A skilled artisan can readily determine the number and interval between boosts.

Table 4 shows a non-limiting example of a sequential immunization protocol using a swarm of HIV1 envelopes optionally in certain embodiments designed as trimers.

| Envelope | Prime | Boost(s) | Boost(s) | Boost(s) |
|---|---|---|---|---|
| CH505.M6 | CH505.M6 | | | |
| CH505.M11 | CH505.M11 as | | | |
| | a nucleic acid | | | |
| | e.g. DNA/MVA | | | |
| | vector and/or | | | |
| | protein | | | |
| CH505w020.14 | | CH505w020.14 | | |
| CH505w030.28 | | CH505w030.28 | | |
| | | as a nucleic acid | | |
| | | e.g. DNA/MVA | | |
| | | vector and/or | | |
| | | protein | | |
| CH505w078.15 | | | CH505w078.15 | |
| CH505w053.31 | | | CH505w053.31 | |
| CH505w030.21 | | | CH505w030.21 as | |
| | | | a nucleic acid e.g. | |
| | | | DNA/MVA | |
| | | | vector and/or | |
| | | | protein | |
| CH505w078.33 | | | | CH505w078.33 |
| CH505w053.36 | | | | CH505w053.36 |
| CH505w100.B6 | | | | CH505w100.B6 |
| | | | | as a nucleic acid |
| | | | | e.g. DNA/MVA |
| | | | | vector and/or |
| | | | | protein |

A skilled artisan can readily determine the number and interval between boosts.

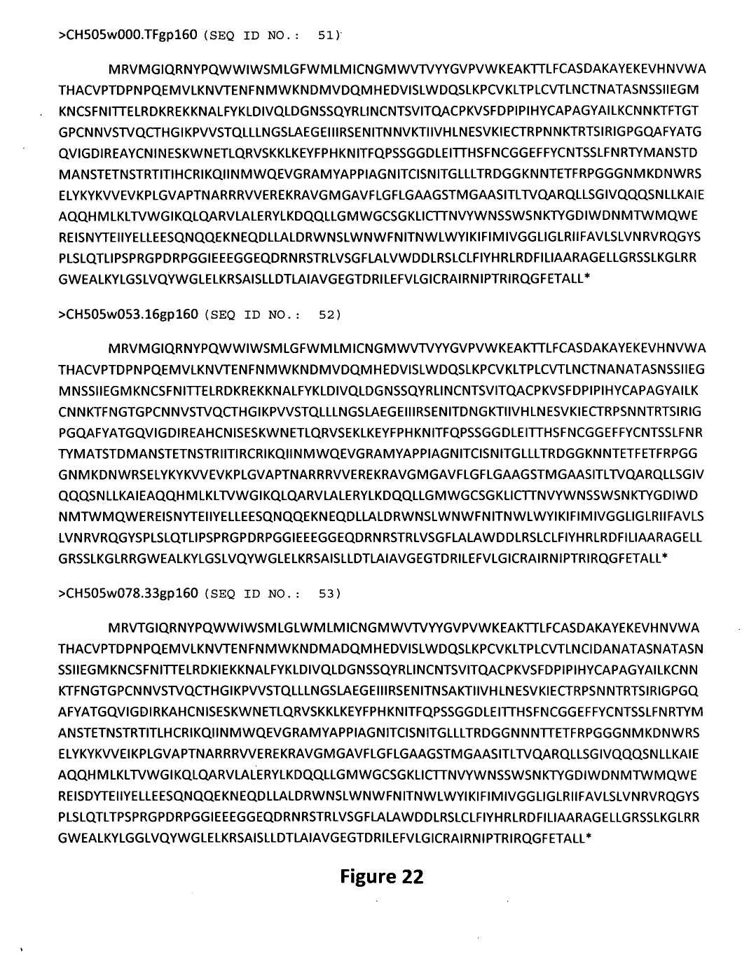

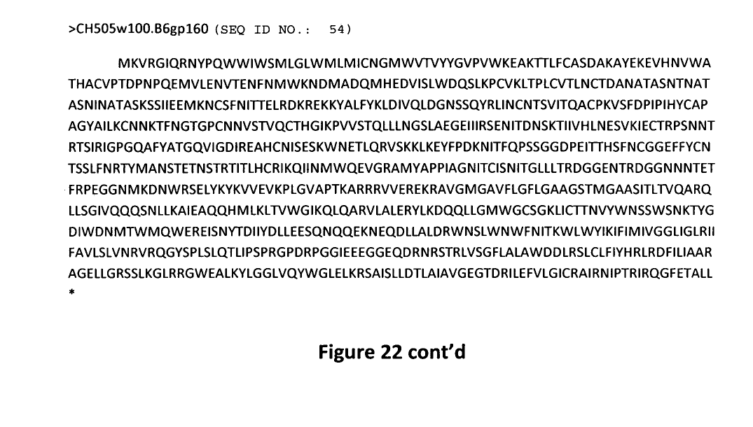

Table 5 shows a non-limiting example of a sequential immunization protocol using a swarm of HIV1 envelopes from CH505 optionally in certain embodiments designed as trimers.

| Envelope | Prime | Boost(s) | Boost(s) | Boost(s) |
|---|---|---|---|---|
| M11 and the | M11 and the | | | |
| T/F | T/F as a nucleic | | | |
| | acid e.g. | | | |
| | DNA/MVA | | | |
| | vector and/or | | | |
| | protein | | | |
| week 20.14 and | | week 20.14 and | | |
| 30.28 | | 30.28 as a | | |
| | | nucleic acid e.g. | | |
| | | DNA/MVA | | |
| | | vector and/or | | |
| | | protein | | |
| week 78.15 and | | | week 78.15 and | |
| 78.33 | | | 78.33 as a nucleic | |
| | | | acid e.g. | |
| | | | DNA/MVA | |
| | | | vector and/or | |
| | | | protein | |
| week 53.16 and | | | | week 53.16 and |
| 100.B6 Envs | | | | 100.B6 Envs as |
| | | | | a nucleic acid |
| | | | | e.g. DNA/MVA |
| | | | | vector and/or |
| | | | | protein |

| Envelope | | |
|---|---|---|
| CH505 | Amino acid sequence | Nucleic acid sequence |
| T/F | FIG. 22 (gp160); See other gp120, | |
| | See e.g. [0068] | |
| M11 | FIG. 17 (D8 gp120; gp160) | FIG. 17 (D8 gp120; gp145; gp160) |
| week 20.14 | | FIG. 17 (D8 gp120; gp145; gp160) |
| week 30.28 | FIG. 17 (D8 gp120; gp160) | FIG. 17 (D8 gp120; gp145; gp160) |
| week 78.15 | FIG. 17 (D8 gp120; gp160) | FIG. 17 (D8 gp120; gp145; gp160) |
| week 78.33 | FIG. 17 (D8 gp120; gp160) | FIG. 17 (D8 gp120; gp145; gp160) |
| week 53.16 | FIG. 17 (D8 gp120; gp160) | FIG. 17 (D8 gp120; gp145; gp160) |
| week 100.B6 | FIG. 17 (D8 gp120; gp160) | FIG. 17 (D8 gp120; gp145; gp160) |

### Example 4: Selection of Ten Early Envelopes

Provided is the approach to selecting a 10-immunogen set from CH505 (See FIG. 24). Here we choose 10 low-diversity variants from the subject early on, rather than down-selecting from a short list of 18, (which you are already making) to represent diversity that appeared through week 160, and includes samples after escape from the mature CH103 mAb.

The hypothesis is that affinity maturation in the presence of antigenic diversity helps select for breadth, allowing it to evolve gradually from a population of Envs selected by clonal autologous neutralization response. But here we would test whether modest variation in the antigen might better stimulate responses that allow the clonal lineage to interact and adapt, while the full range of variation might introduce too much diversity for the developing lineage. For example, a set of Envs with 1 or 2 substitutions in an epitope might reduce affinity, but still allow binding, and the evolving B cell population would be able to adapt. Such variants might allow more “generalists” to evolve. Env variants fully escaped from early lineage clones might be immunologically silent, and less able to draw increased breadth from the B cell clones.

This is essentially like trying a serial version of the swarm vaccine of 100, where we plan on starting with the low-diversity forms, and increase diversity as we vaccinate, but by making these 10 we could try other delivery strategies.

We selected a set of 10 gp120s for use as candidate immunogens. The focus here is on Env diversity at week 30, which coincides with an expansion in heterologous neutralization seen also by antigenic cartography. Unlike the TF and earlier forms, all week 30 sequences contain the V3 glycan shift from 334 to 332.

We identified Env sites to use as criteria for Env selection. The sites were determined by TF loss, neutralization signatures, and contact with the CD4bs and CH103 bnAb (Table 6): (a) At least 80% TF loss through week 160 yielded 36 sites, as described previously. (b) Neutralization signatures for single or PNG sites with q<0.1 for tree-corrected signatures of IC50s below 20 μg/ml, as described previously. (c) The list of contact sites was expanded by one amino acid up- and downstream of each known contact, to include a slightly larger neighborhood of contact sites. These 66 HXB2 sites grew to 71 sites when mapped onto the CH505 Env alignment. When reviewed for polymorphisms, 28 of these sites vary in CH505 over the sampling period.

CTL responses were mapped and found one ELISpot positive peptide on the C-terminus of the V4 loop, sites 409-418, EGSDTITLPC in HXB2, NSTRTITIHC in CH505. CTL epitope variants are identified among selected sites in Table 5.

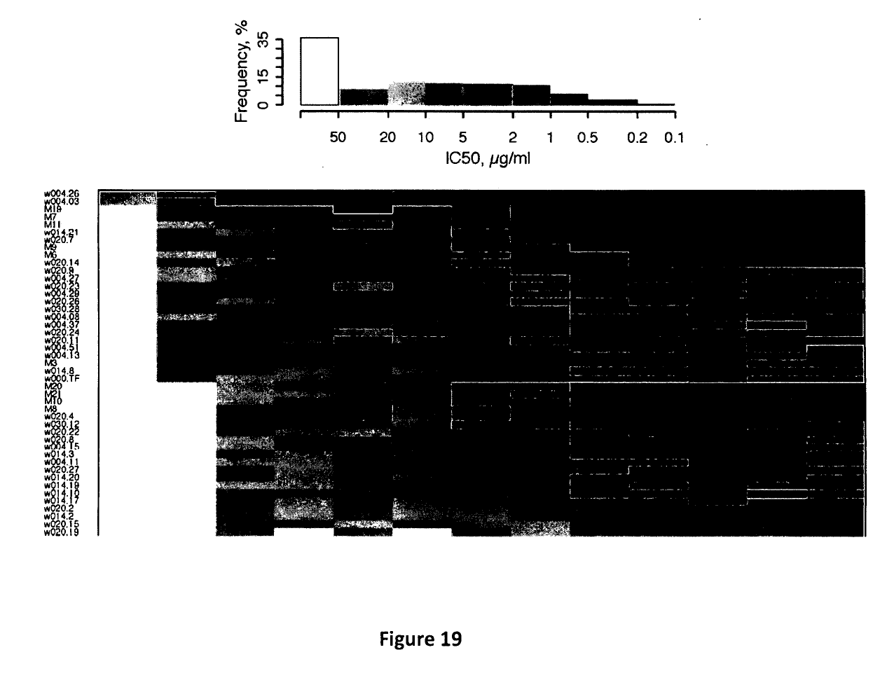

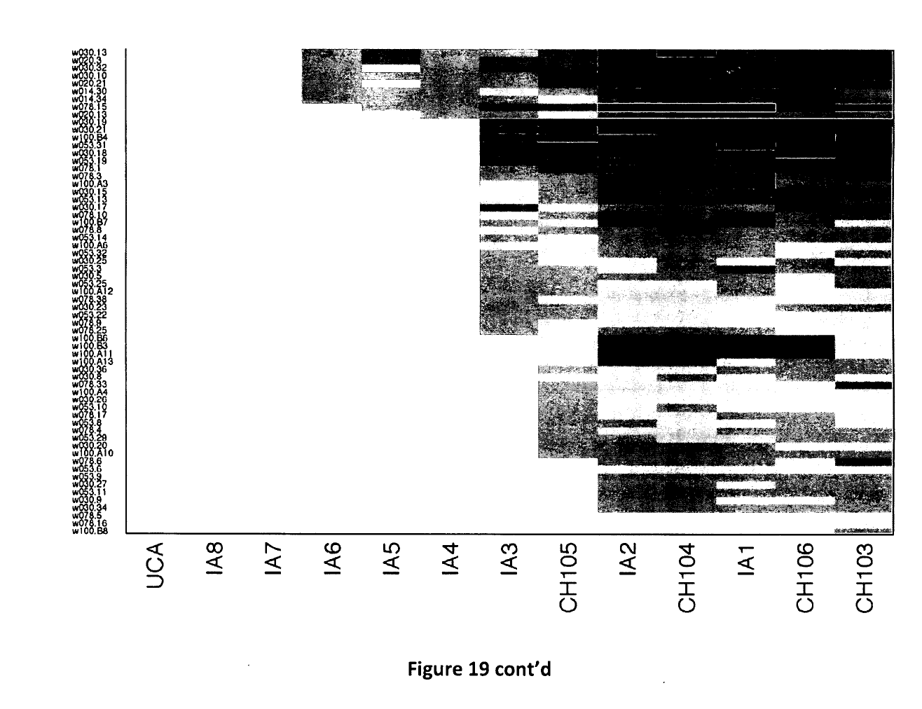

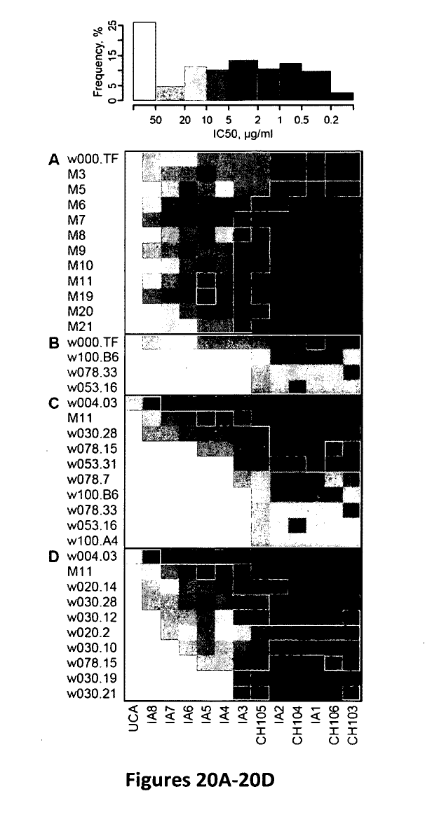

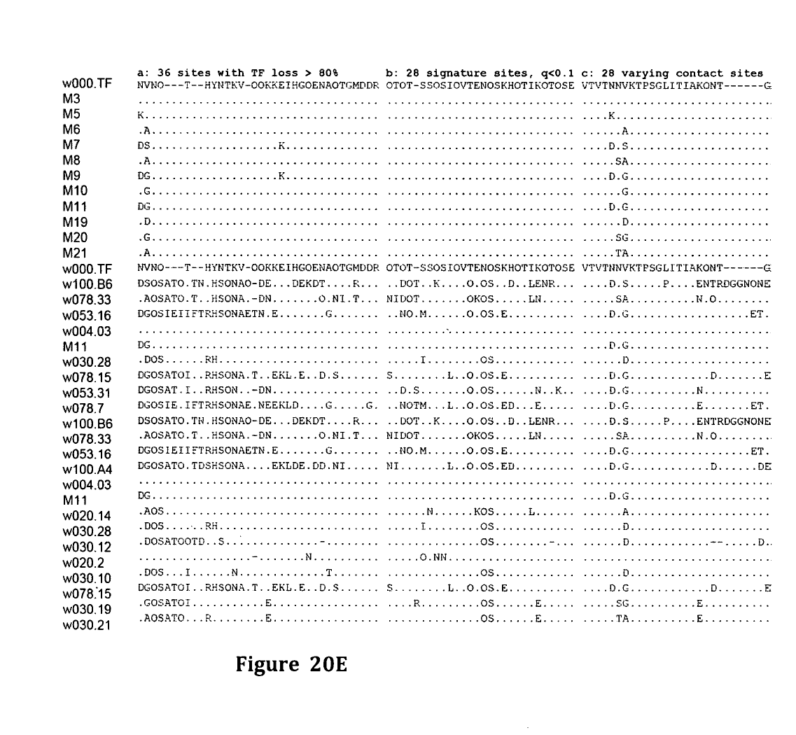

Neutralization sensitivity of autologous Envs to mAbs in the CH103 lineage further informs selection of 10 Envs (FIG. 19). Comparing selected Envs with concatenated sites (FIG. 20) allows selection for incremental progression of mAb sensitivities (FIG. 20D). An abrupt transition between neutralization sensitivity to IA7 and IA3 limits available Envs from week 30 (FIG. 19), perhaps because of the mAb discontinuity induced by a shift in light-chain usage from UCA to IA2 light chain associations with IA4 and IA3 heavy chains, respectively (i.e. IA4 mAb is 14 V_{H} and UCA V_{L}; IA3 mAb is V_{H} I3 with V_{L} I2).

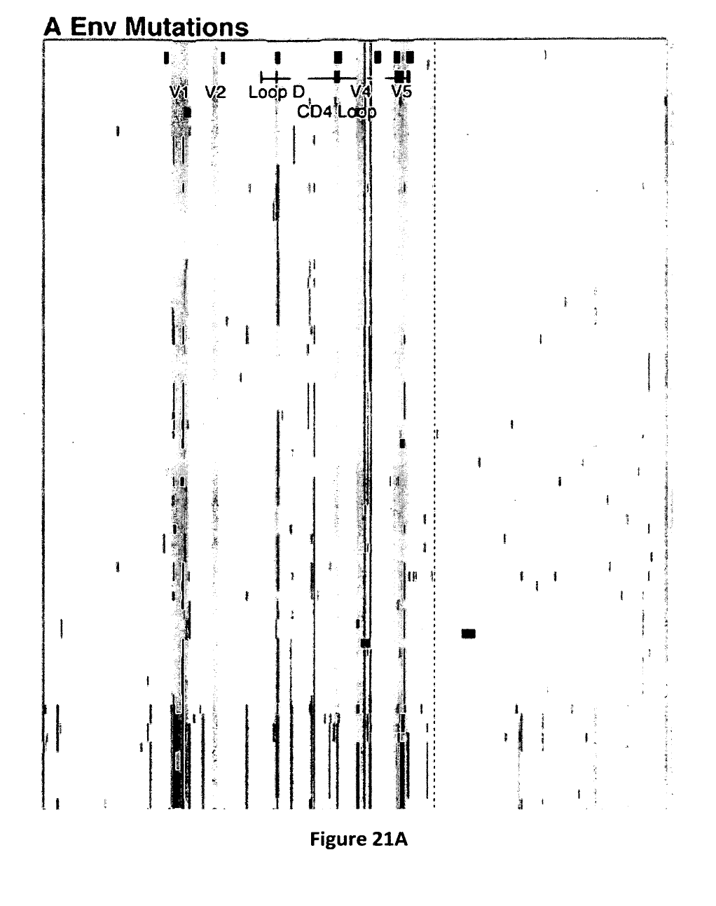

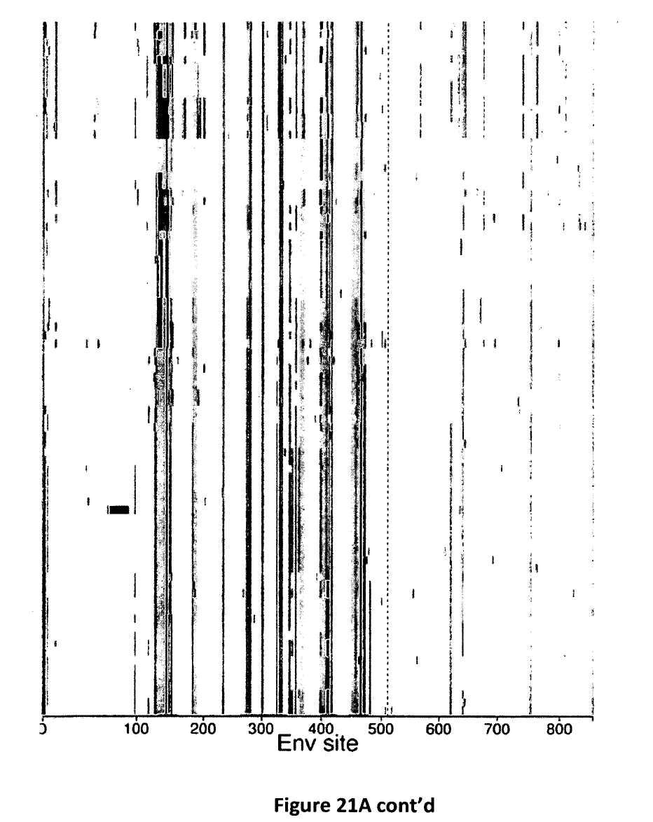

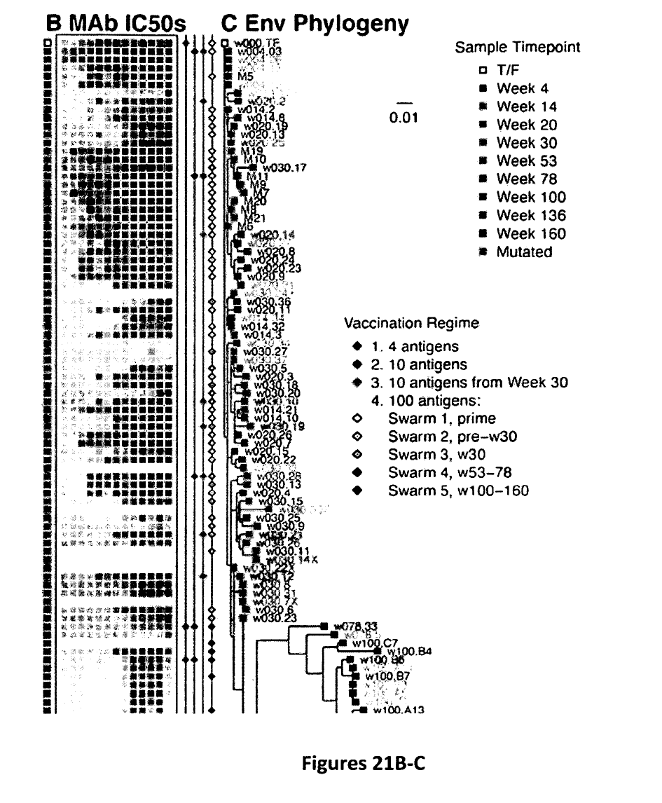

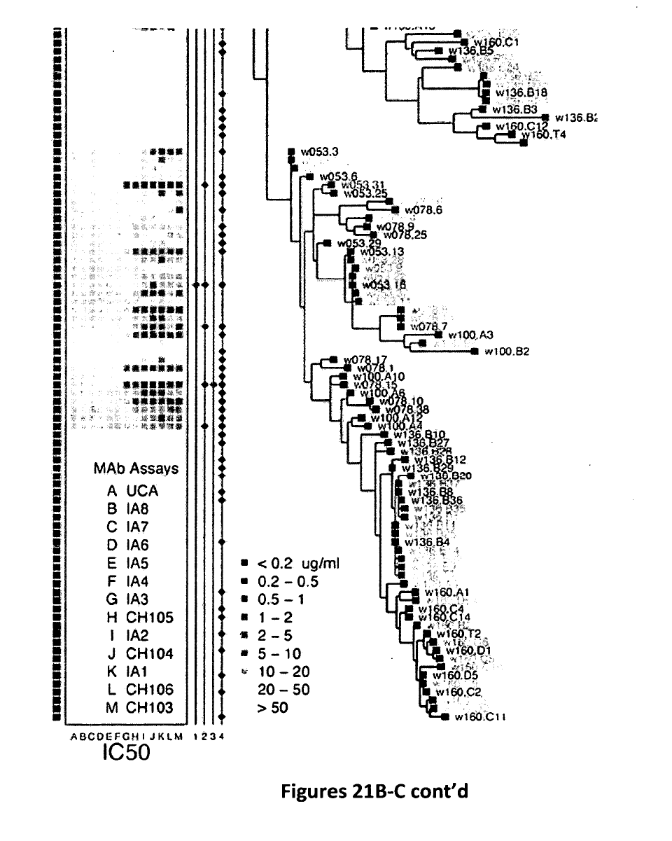

CH505 Env diversity and neutralization to the CH103 lineage mAbs, together with the distributions of proposed sets of 4, 10 (new and in preparation), and 100 antigens are all compared by established methods in FIGS. 21A-21C.

TABLE 6
Alignment columns in Env “hot-spot” concatamer summaries.

| | | | | |
|---|---|---|---|---|
| Col | HXB2 | AA | CH505 | Feature |

a: 36 sites with TF loss >80%

| | | | | |
|---|---|---|---|---|
| 1 | 279 | D | N | Loop D |
| 2 | 281 | A | V | Loop D |
| 3 | 332 | O | N | PGT121 |
| 4 | 334 | S | O | 2G12 |
| 5 | 144+ | — | — | V1 |
| 6 | 144+ | — | — | V1 |
| 7 | 144+ | — | — | V1 |
| 8 | 413 | T | T | V4/CTL |
| 9 | 465 | S | — | V5 |
| 10 | 464 | E | — | V5 |
| 11 | 417 | P | H | V4/CTL |
| 12 | 330 | H | Y | V3 |
| 13 | 300 | N | N | V3 |
| 14 | 234 | O | T | 8ANC195 |
| 15 | 302 | N | K | V3 |
| 16 | 756 | I | V | gp41 |
| 17 | 463+ | — | — | V5 |
| 18 | 398 | S | O | V4 |
| 19 | 133 | D | O | V1 |
| 20 | 460 | N | K | V5 |
| 21 | 347 | S | K | |
| 22 | 275 | V | E | Loop D |
| 23 | 151 | K | I | V1 |
| 24 | 356 | O | H | |
| 25 | 471 | G | G | beta24 |
| 26 | 147 | M | O | V1 |
| 27 | 640 | S | E | gp41 |
| 28 | 462 | N | N | V5 |
| 29 | 145 | G | A | V1 |
| 30 | 130 | K | O | |
| 31 | 132 | T | T | V1 |
| 32 | 620 | E | G | gp41 |
| 33 | 4 | K | M | SignalPep |
| 34 | 325 | N | D | V3 |
| 35 | 185 | D | D | V2 |
| 36 | 412 | D | R | V4/CTL |

b: 28 signature sites, g < 0.1

| | | | | |
|---|---|---|---|---|
| 1 | 130 | K | O | |
| 2 | 132 | T | T | V1 |
| 3 | 133 | D | O | V1 |
| 4 | 135 | K | T | V1 |
| 5 | 137 | D | — | V1 |
| 6 | 146 | R | S | V1 |
| 7 | 148 | I | S | V1 |
| 8 | 147 | M | O | V1 |
| 9 | 149 | M | S | V1 |
| 10 | 151 | K | I | V1 |
| 11 | 160 | O | O | PG9 |
| 12 | 200 | V | V | |
| 13 | 234 | O | T | 8ANC195 |
| 14 | 328 | Q | E | V3 |
| 15 | 332 | O | N | PGT121 |
| 16 | 334 | S | O | 2G12 |
| 17 | 336 | A | S | |
| 18 | 347 | S | K | |
| 19 | 356 | O | H | |
| 20 | 358 | T | O | |
| 21 | 360 | I | T | |
| 22 | 416 | L | I | V4/CTL |
| 23 | 460 | N | K | V5 |
| 24 | 461 | S | O | V5 |
| 25 | 463 | O | T | V5 |
| 26 | 743 | D | O | Kennedy |
| 27 | 745 | S | S | Epitope |
| 28 | 831 | E | E | LLP-1 |

c: 28 varying contacts

| | | | | |
|---|---|---|---|---|
| 1 | 127 | V | V | CD4 |
| 2 | 128 | S | T | CD4 |
| 3 | 255 | V | V | |
| 4 | 278 | T | T | |
| 5 | 279 | D | N | Loop D |
| 6 | 280 | N | N | Loop D |
| 7 | 281 | A | V | Loop D |
| 8 | 282 | K | K | Loop D |
| 9 | 283 | T | T | Loop D |
| 10 | 363 | Q | P | |
| 11 | 365 | S | S | |
| 12 | 367 | G | G | |
| 13 | 369 | P | L | CD4 |
| 14 | 371 | I | I | CD4 |
| 15 | 372 | V | T | |
| 16 | 424 | I | I | |
| 17 | 433 | A | A | |
| 18 | 460 | N | K | V5 |
| 19 | 461 | S | O | V5 |
| 20 | 462 | N | N | V5 |
| 21 | 463 | N | T | V5 |
| 22 | 463+ | — | — | V5 |
| 23 | 463+ | — | — | V5 |
| 24 | 463+ | — | — | V5 |
| 25 | 463+ | — | — | V5 |
| 26 | 463+ | — | — | V5 |
| 27 | 464+ | E | — | V5 |
| 28 | 471 | G | G | Beta24 |

### Example 5: Non-Human Primate Studies

NHP 79: CH505T/F gp120 envelope in GLA/SE. NHP 85: CH505T/F gp140 envelope in GLA/SE. This compares gp140 with gp120 induced antibodies.

NHP study of CH505T/F gp120 with GCN4 CH505 T/F in GLA/SE.

NHP study of CH505T/F gp120 with GCN4 CD40L CH505 T/F in GLA/SE.

NHP study of CH505T/F gp120 with GCN4 CD40L CH505 T/F in ALUM.

NHP study of CH505 T/F gp120 with GCN4 CD40L CH505 T/F=-HIS tag with liposomes in ALUM.

NHP study of M6 then rest of production 10 (Table 4) gp120 in sequence gp120 GNC4 CD40L CH505 trimers with ALUM or GLA/SE (depends on antigenicity).

NHP study of M6 then rest of production 10 (Table 4) gp120 in sequence gp120 GNC4 CD40L CH505 trimers in ALUM or GLA/SE (depends on antigenicity), with a dose of chloloquine orally each day 10 days before each immunization and then a dose of CD25 Ab 5 days after each immunization. See U.S. Application Ser. No. 62/056,583 (filed Sep. 28, 2014), which content is herein incorporated by reference in its entirety.

The contents of all documents and other information sources cited herein are herein incorporated by reference in their entirety.

### Example 6 Selection of Eight Envelopes for Use as a Vaccine

Over the past 5 years the HIV vaccine development field has realized that immunization with a single HIV envelope protein is not going to be successful for induction of broadly neutralizing antibodies (bnAbs) (Mascola and Haynes, 2013). Moreover, the biology of broad neutralizing antibodies has also become clearer, with evidence for a role of host immune tolerance control mechanisms in limiting the induction of bnAbs (reviewed in (Haynes and Verkoczy, 2014; Mascola and Haynes, 2013). While the role of the structure of the Env immunogens is undoubtedly important, i.e. the Env must contain sufficiently native bnAb epitopes to bind in nM affinities to the unmutated common ancestor (naïve B cell receptors) of bnAb lineages (Haynes et al., 2012; Jardine et al., 2013), whether a native trimer is needed for this purpose or if a highly antigenic Env subunit will suffice is as yet unknown. Studies in mice in basic B cell biology have demonstrated that what is important for B cell survival in the germinal center (GC) is the affinity of the immunogen for the GC B cell receptor (Dal Porto et al., 2002; Shih et al., 2002).

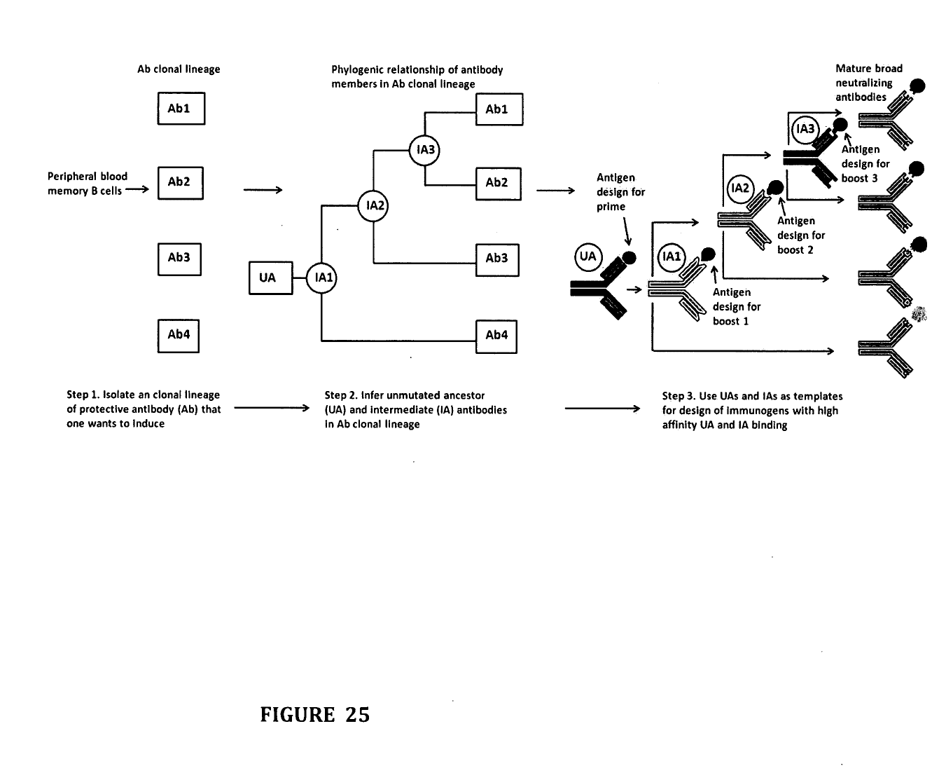

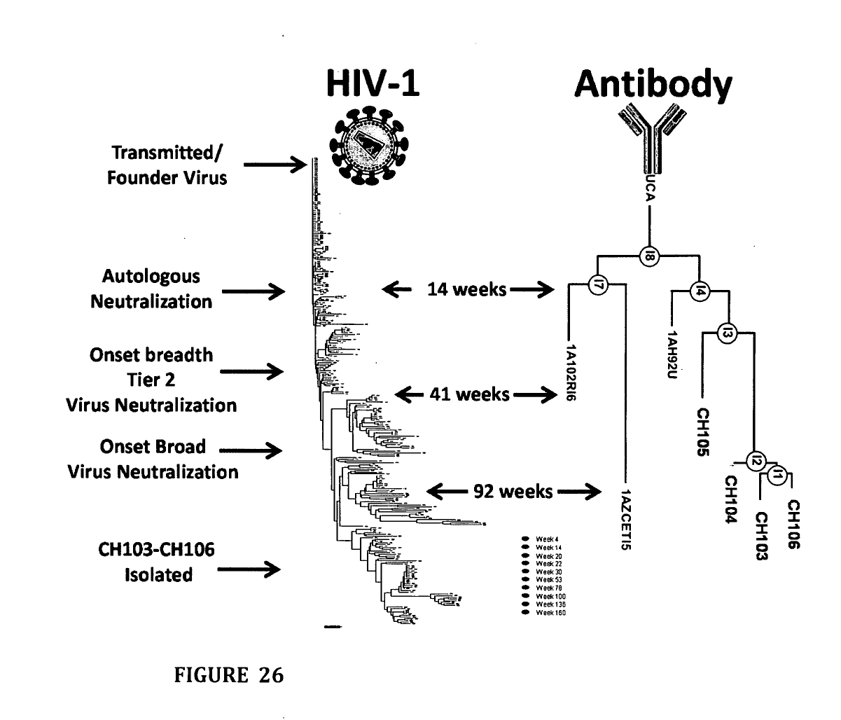

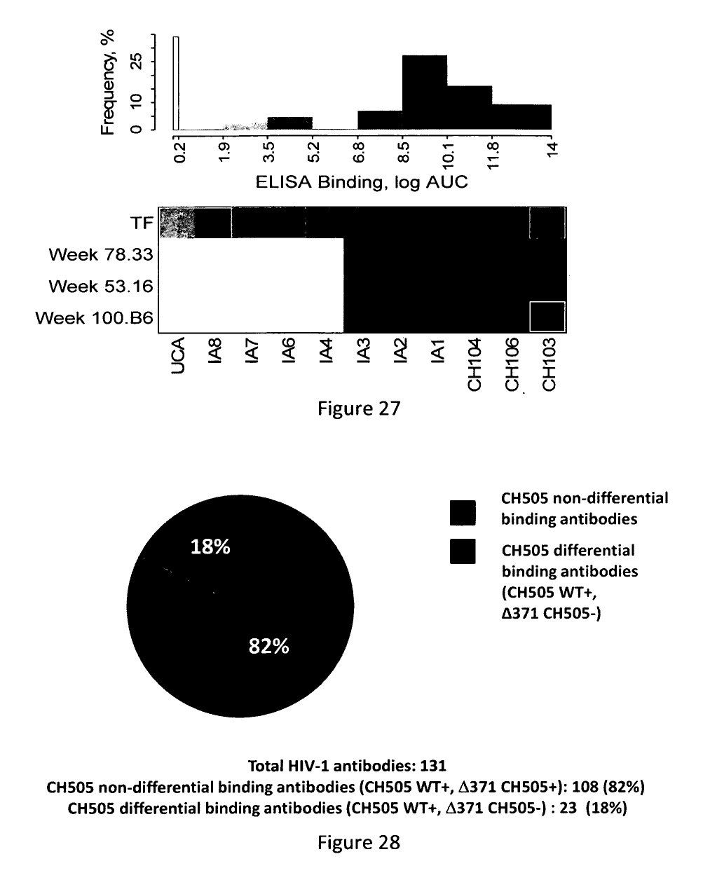

Thus, the concept of B cell lineage immunogen design has arisen, whereby lineages of bnAbs are elucidated, and Envs chosen for sequential immunizations based on optimized affinity of Env immunogens for BCR at sequential steps of the affinity maturation pathway of bnAb lineages (Haynes et al., 2012) (FIG. 25). While Envs have been designed for reacting with UCAs of heterologous bnAb lineages (Jardine et al., 2013; McGuire et al., 2013), we have taken the approach of defining in select HIV-infected individuals who make bnAbs the natural sequence of Envs that in that person induced the bnAb lineage, in order to take the guessing out of Env selection. Thus, from African individual CH505, we isolated both sequential Envs and bnAbs over time and mapped the co-evolution of the CH103 CD4 binding site bnAb lineage (Liao et al., 2013) (FIG. 26, FIGS. 21A-21C). We first picked 4 envelopes and produced them as gp120s and determined if they reacted with the UCA, intermediate antibodies and mature antibodies of the CH103 bnAb lineage. FIG. 27 shows the reactivity of these Env gp120s with the CH103 lineage as measured in ELISA with data shown as log area under the curve (AUC). Of 30 CH505 Env mutants screened, we found 4, the transmitted/founder (T/F) Env, the week 78.33 Env, the week 53.16 Env and the week 100.B6 Env, that optimally reacted best with each step of the CH103 lineage (FIG. 27). In surface plasmon resonance assays, the T/F Env gp120 reacted with the UCA of the CH103 lineage with a K_{D} of ˜200 nM.

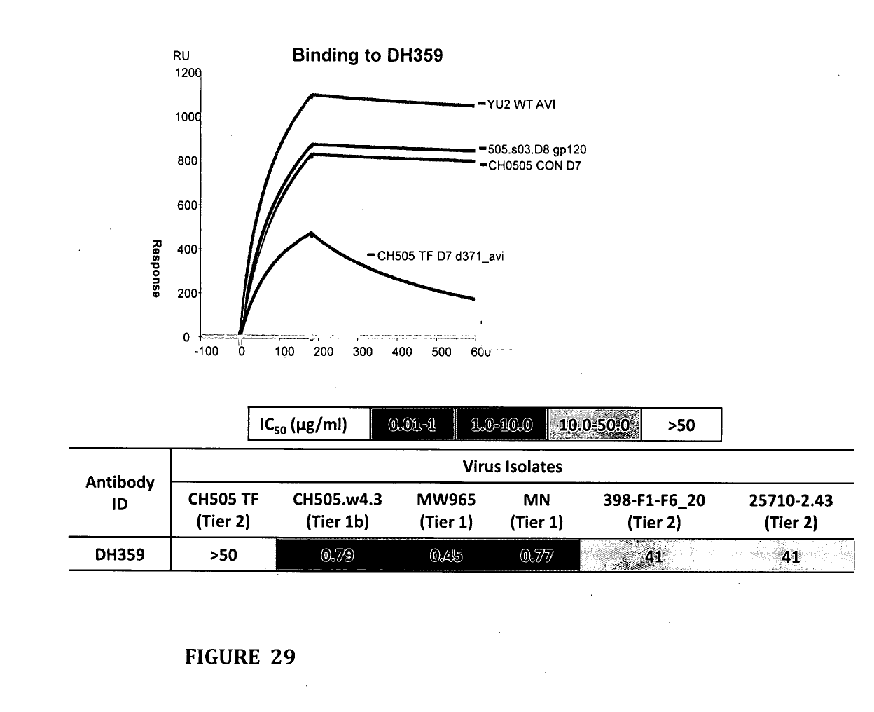

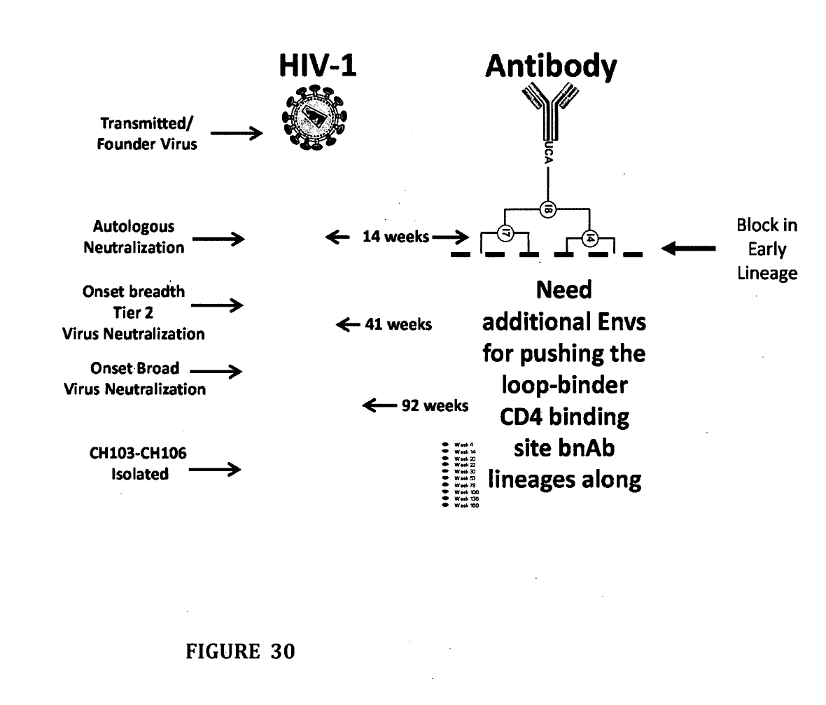

Using this 4-valent sequential immunogen of CH505 Envs in rhesus macaques, we determined if we had induced triggering of the UCA of a lineage capable of going on to bnAb evolution by the following criteria of the CH103 UCA characteristics: 1) no neutralization of the tier 2 CH505 T/F virus; 2) neutralization of the tier 1B CH505 T/F variant 4.3; 3) differentially binds to the CH505 T/F gp120 versus the mutated CH505 gp120 with a deletion of isoleucine at 371; 4) the lineage precursors are subdominant to other CH505 Env-binding lineages. When we isolated 131 Env reactive antibodies from rhesus macaques immunized with CH505 4-valent sequential Envs, we indeed did isolate 23/131 (18%) of antibodies with this profile (FIG. 28). FIG. 29 shows the results of one such antibody DH359. FIG. 30 shows how far we believe we drove such a lineage and the need for additional Envs to complete the lineage induction.

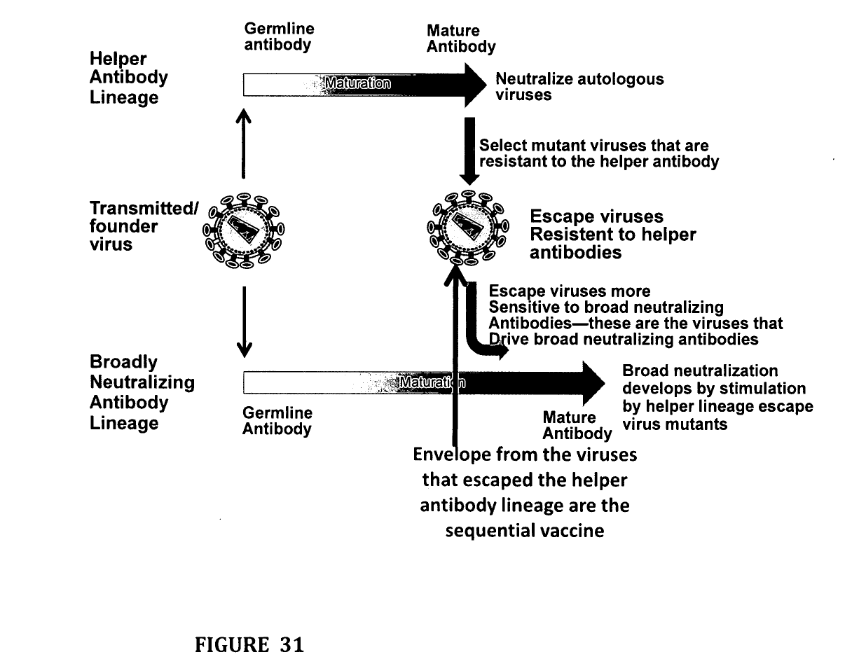

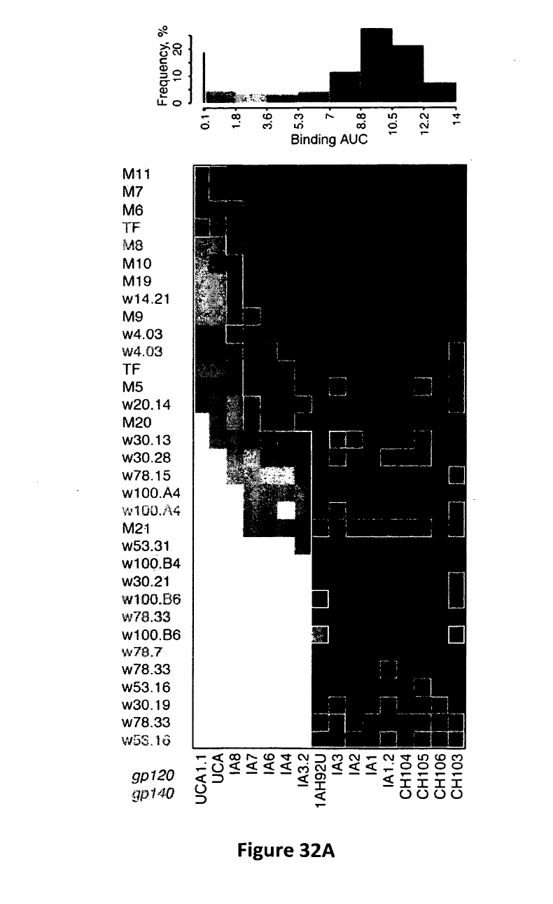

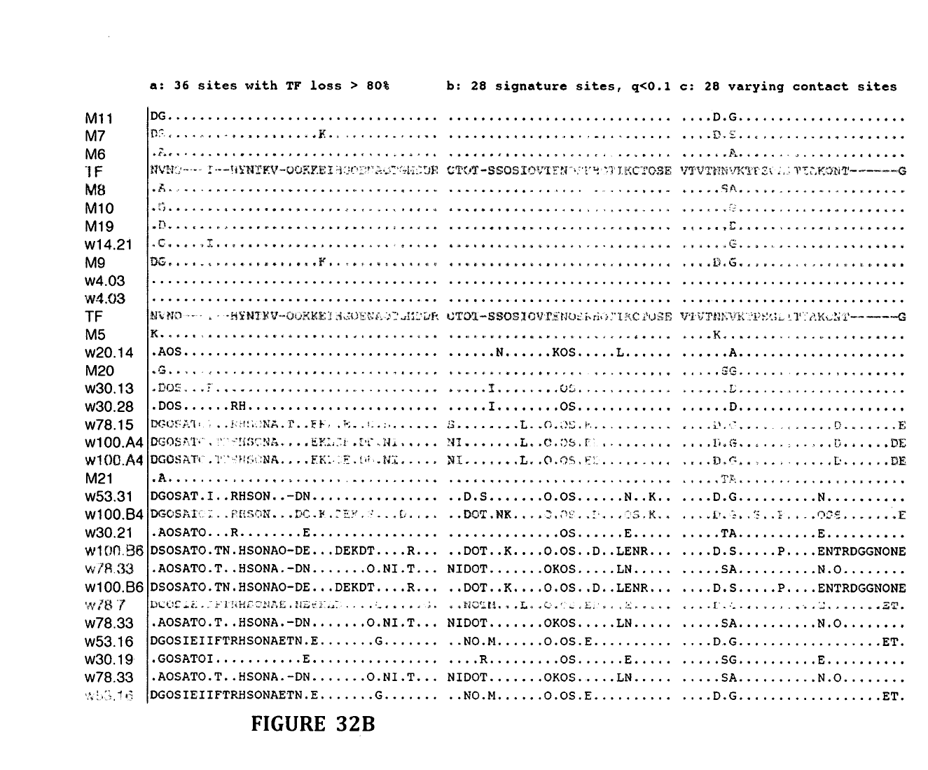

Our next question was to define a new strategy for selecting additional Envs that may have been involved in inducing the CH103 lineage. We found such a strategy by making all the Env mutants in the contact regions between the CH103 antibody and HIV Env (Liao et al., 2013). In doing so, we found a series of Env mutants that were resistant to the CH103 bnAb lineage and therefore were selected by the bnAb lineage CH103. However, we also found a set of Env mutants, the gp120 loop D mutants that were not resistant to CH103 neutralization but rather were more potently neutralized by the CH103 bnAb than the T/F wild-type virus (Gao et al., 2014). Thus, these mutants could not have been selected by the CH103 bnAb lineage and suggested the existence of a second neutralizing lineage that neutralized the T/F virus but did not neutralize the loop D CH505 mutant viruses. We went on to isolate such a lineage, and demonstrate the presence of this “helper” lineage that selected Env escape mutants that drove the CH103 bnAb lineage (Gao et al., 2014) (FIG. 31). The importance of this observation for vaccine design is that in this manner, we clearly defined a set of Envs that participated in driving the CH103 lineage and should be included in the sequential vaccine (Gao et al., 2014). Thus, we expressed a number of sequential gp120 Envs from CH505 mutant viruses that were resistant to the “helper lineage” but sensitive to the CH103 bnAb lineage, and tested them in binding assays with the antibodies of the CH103 bnAb lineage (FIG. 32).

From this analysis, we chose 8 sequential Envs that had the highest likelihood of binding well to the CH103 lineage and “filled in” the space of binding to the UCA, IA8, IA7, IA6 and IA4 that was not present in FIG. 27. The new Envs are the loop D mutant Ml 1, and the natural loop D mutants from week 20.14, week 30.28, and week 78.15 Env gp120s. The M11 was chosen because it bound better to the UCA than the T/F itself.

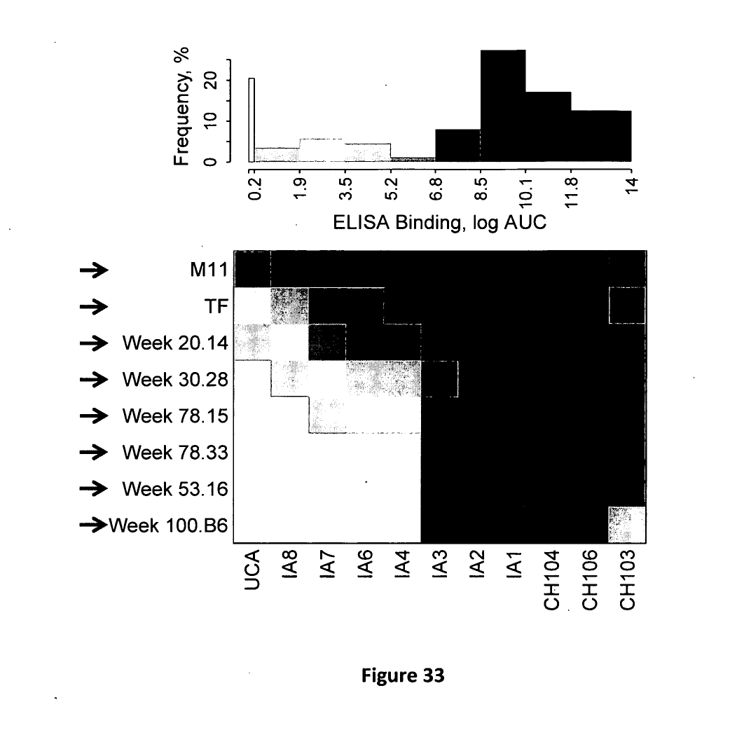

The importance of this analysis is as follows. The red arrows in FIG. 33 indicate the 4 CH505 Envs (TF, Week 78.33, Wee 53.16, Week 100.B6) currently under production. A human phase I trial with these 4 Envs administered either in sequence or as a swarm will be tested in the HVTN starting in ˜1 year to determine if CH103-like lineage can be initiated in humans as was initiated in primates. We should have more success even than in rhesus macaques since we can target the specific VH4-59, 13-1 germlines that are present in humans but for which (at least for the VH4-59) there is only an imperfect ortholog in rhesus.

The black arrows indicate the 4 CH505 Envs (D mutant M11, and the natural loop D mutants from week 20.14, week 30.28, and week 78.15 Env) that we propose to make to complete this sequence of Env immunization to further drive loop binding CD4bs bnAbs. We propose to have these 4 loop D mutants available at about the time that the data from the first human trials with the first 4 CH505 Envs has been completed.

We propose a Phase I trial administering all 8 of the envs (FIG. 33) in various combinations. In certain embodiments the prime is with Ml 1 and the T/F Env, then boost with week 20.14 and 30.28, then boost with week 78.15 and 78.33, then final boost with week 53.16 and 100.B6 Envs. In other embodiments, the with M11 and the T/F Env, then boost with a combination of any of the other envelopes. Given the data that we can initiate the CH103-like lineage in NHPs and the direct evidence we have in vivo now from the delineation of loop D mutants (Gao et al., 2014), the addition of the 4 loop D mutants is a very rational next step in the process of induction of bnAbs in humans.

### BIBLIOGRAPHY FOR EXAMPLE 6

Dal Porto, J. M., A. M. Haberman, G. Kelsoe, and M. J. Shlomchik. 2002. Very low affinity B cells form germinal centers, become memory B cells, and participate in secondary immune responses when higher affinity competition is reduced. The Journal of experimental medicine 195:1215-1221.
Gao, F., M. Bonsignori, H. X. Liao, A. Kumar, S. M. Xia, X. Lu, F. Cai, K. K. Hwang, H. Song, T. Zhou, R. M. Lynch, S. M. Alam, M. A. Moody, G. Ferrari, M. Berrong, G. Kelsoe, G. M. Shaw, B. H. Hahn, D. C. Montefiori, G. Kamanga, M. S. Cohen, P. Hraber, P. D. Kwong, B. T. Korber, J. R. Mascola, T. B. Kepler, and B. F. Haynes. 2014. Cooperation of B cell lineages in induction of HIV-1-broadly neutralizing antibodies. Cell 158:481-491.
Haynes, B. F., G. Kelsoe, S. C. Harrison, and T. B. Kepler. 2012. B-cell-lineage immunogen design in vaccine development with HIV-1 as a case study. Nature biotechnology 30:423-433.
Haynes, B. F., and L. Verkoczy. 2014. AIDS/HIV. Host controls of HIV neutralizing antibodies. Science 344:588-589.
Jardine, J., J. P. Julien, S. Menis, T. Ota, O. Kalyuzhniy, A. McGuire, D. Sok, P. S. Huang, S. MacPherson, M. Jones, T. Nieusma, J. Mathison, D. Baker, A. B. Ward, D. R. Burton, L. Stamatatos, D. Nemazee, I. A. Wilson, and W. R. Schief. 2013. Rational HIV immunogen design to target specific germline B cell receptors. Science 340:711-716.
Liao, H. X., R. Lynch, T. Zhou, F. Gao, S. M. Alam, S. D. Boyd, A. Z. Fire, K. M. Roskin, C. A. Schramm, Z. Zhang, J. Zhu, L. Shapiro, N. C. S. Program, J. C. Mullikin, S. Gnanakaran, P. Hraber, K. Wiehe, G. Kelsoe, G. Yang, S. M. Xia, D. C. Montefiori, R. Parks, K. E. Lloyd, R. M. Scearce, K. A. Soderberg, M. Cohen, G. Kamanga, M. K. Louder, L. M. Tran, Y. Chen, F. Cai, S. Chen, S. Moquin, X. Du, M. G. Joyce, S. Srivatsan, B. Zhang, A. Zheng, G. M. Shaw, B. H. Hahn, T. B. Kepler, B. T. Korber, P. D. Kwong, J. R. Mascola, and B. F. Haynes. 2013. Co-evolution of a broadly neutralizing HIV-1 antibody and founder virus. Nature 496:469-476.
Mascola, J. R., and B. F. Haynes. 2013. HIV-1 neutralizing antibodies: understanding nature's pathways. Immunological reviews 254:225-244.
McGuire, A. T., S. Hoot, A. M. Dreyer, A. Lippy, A. Stuart, K. W. Cohen, J. Jardine, S. Menis, J. F. Scheid, A. P. West, W. R. Schief, and L. Stamatatos. 2013. Engineering HIV envelope protein to activate germline B cell receptors of broadly neutralizing anti-CD4 binding site antibodies. The Journal of experimental medicine 210:655-663.
Shih, T. A., E. Meffre, M. Roederer, and M. C. Nussenzweig. 2002. Role of BCR affinity in T cell dependent antibody responses in vivo. Nature immunology 3:570-575.

What is claimed is:

1. A composition comprising any one of the polypeptides M11, T/F Env, week 20.14, week 30.28, week 78.15, week 78.33, week 53.16, week 100.B6 Envs, or a combination thereof.

2. A composition comprising any one of the polypeptides M11, T/F Env, week 20.14, week 30.28, week 78.15, week 78.33, week 53.16, week 100.B6 Envs, wherein the polypeptide further comprises trimerization domain.

3. The composition of claim 2, wherein the trimerization domain is GCN4.

4. A composition comprising a nucleic acid encoding any one of the polypeptides of claim 1-3.

5. The composition of claim 1 or 2, wherein the HIV-1 envelopes are M11 and T/F Env. The composition of claim 1 or 2, wherein the HIV-1 envelopes are week 20.14 and week 30.28. The composition of claim 1 or 2, wherein the HIV-1 envelopes are week 78.15 and week 78.33. The composition of claim 1 or 2, wherein the HIV-1 envelopes are week 53.16 and week 100.B6 Envs.

6. The composition of any one of claims 1-5 further comprising an adjuvant.

7. A composition comprising the polypeptides M11, T/F Env, week 20.14, week 30.28, week 78.15, week 78.33, week 53.16, and week 100.B6 Envs.

8. The composition of claim 7, wherein at least one of the polypeptides further comprises a trimerization domain.

9. The composition of claim 8, wherein the trimerization domain is GCN4.

10. A method of inducing an immune response in a subject comprising administering the composition of any one of claims 1-9 in an amount sufficient to induce an immune response.

11. The method of claim 10 further comprising administering chloloquine before each immunization (in certain embodiments, chloloquine is administered for about 10 days before each immunization).

12. The method of claim 10 further comprising administering anti-CD25 antibody after each immunization (in certain embodiments, anti-CD25 antibody is administered for about 5 days after each immunization).

13. The method of claim 11 further comprising administering anti-CD25 antibody after each immunization (in certain embodiments, anti-CD25 antibody is administered for about 5 days before each immunization).

14. The method of claim 10, wherein the composition comprises a nucleic acid, a protein or any combination thereof.

15. The method of claim 14, wherein the nucleic acid encoding the envelope is operably linked to a promoter inserted in an expression vector.

16. The method of claim 14, wherein the protein is recombinant.

17. The method of claim 14, wherein the composition is administered as a prime, a boost, or both.

18. The method of claim 14, wherein the composition is administered as a multiple boosts.

19. The method of claim 10-18, wherein the composition further comprises an adjuvant.
